(19) United States
(12) Patent Application Publication
(10) Pub. No.: US 2023/0193212 A1
(43) Pub. Date: Jun. 22, 2023

(54) TREATMENT FOR NEURODEGENERATIVE DISEASES

(71) Applicants: ORCHARD THERAPEUTICS (EUROPE) LIMITED, London (GB); UNIVERSITA DEGLI STUDI DI PADOVA, Padova (IT)

(72) Inventors: Alessandra Biffi, Padua (IT); Yuri Ciervo, Padova (IT)

(73) Assignees: ORCHARD THERAPEUTICS (EUROPE) LIMITED, London (GB); UNIVERSITA DEGLI STUDI DI PADOVA, Padova (IT); UNIVERSITA DEGLI STUDI DI PADOVA, Padova (IT)

(21) Appl. No.: 17/923,482

(22) PCT Filed: May 6, 2021
(86) PCT No.: PCT/GB2021/051103

§ 371 Date: Nov. 4, 2022

(30) Foreign Application Priority Data
May 6, 2020 (IT) 102020000010138

(51) Int. Cl. C12N 5/10; C07K 14/52; C12N 15/86; A61P 25/28; A61K 35/28

(52) U.S. Cl. CPC C12N 5/10; C07K 14/52; C12N 15/86; A61P 25/28; A61K 35/28; C12N 2740/16043

(57) ABSTRACT

The invention relates to the field of gene therapy and more specifically to hematopoietic stem and progenitor cells that have been genetically modified to express functional progranulin. Suitably, HSPCs that have gene edited using CRISPR/Cas technology, or have been transduced with lentiviral gene delivery vehicles. The invention also relates to the use of such modified HSPCs in therapy. In particular, the invention relates to use of the modified progranulin expressing HSPCs in the prevention or treatment of a neurodegenerative disease mediated by dysfunctional progranulin expression, such as frontotemporal dementia (FTD).

### FIELD OF THE INVENTION

The invention relates to haematopoietic stem and progenitor cells (HSPCs) modified to express progranulin and methods for treating neurodegenerative diseases such as frontotemporal dementia (FTD) using haematopoietic stem cell gene therapy (HSCGT) with such modified HSPCs.

### INTRODUCTION

Frontotemporal dementia (FTD) is the most common neurodegenerative disorder in individuals under age 60 with currently no treatment or cure (e.g. see Bang et al., Lancet. 386:1672-1682, 2015; Petkau and Leavitt. Trends Neurosci 37:388-398, 2014). Abnormalities in the behavior, personality and language (Cruts et al. Trends Genet 24:186-194, 2008) are common features of FTD carriers. Atrophy of the frontal and anterior temporal brain lobes are the pathological tracts, along with gliosis, swollen neurons, micro-vacuolation, and ubiquitin, tau, TAR DNA-binding protein 43 (TDP-43), or fused-in-sarcoma inclusion bodies, detected by post mortem analysis of brain tissue (Bang et al., Lancet. 386:1672-1682, 2015; Neary et al., Lancet Neurol 4:771-780, 2005). Death usually occurs within 6-8 years of diagnosis and currently there is no cure or treatment.

Mutations in the progranulin gene (GRN) accounts for 5-26% of FTD cases in different populations (e.g. see: Baker et al. Nature 442:916-919, 2006; Cruts et al. Nature 442:920-924, 2006; Gass et al. Hum Mol Genet 15:2988-3001, 2006). The pathophysiology of FTD is unclear but is related to progranulin's roles in lysosomal (e.g. Smith et al, Am J Hum Genet 90:1102-1107, 2012), neurotrophic (Van Damme et al. J Cell Biol 181:37-41, 2008), and anti-inflammatory functions (e.g. Yin et al. J Exp Med 207:117-128, 2010), together with increased complement activation and altered synaptic pruning (Lui et al., Cell 165:921-935, 2016). Expressed by a plethora of cells, such as neurons, microglia, macrophages, epithelial cells, and adipocytes, progranulin localizes to lysosomes in cells (e.g. Tanaka et al., Neuroscience 250:8-19, 2013 and Zhou X, et al. J Cell Biol 210:991-1002, 2015). As a secreted protein, it can be found in cerebrospinal fluid (De Riz et al., Neurosci Lett 469:234-236, 2010; and Feneberg et al., J Neural Transm (Vienna) 123:289-296, 2016) and blood (Martens et al. J Clin Invest 122:3955-3959, 2012; and, Feneberg et al., supra).

The majority of FTD pathogenetic GRN alleles are nonsense and frameshift mutations, while total GRN ablation in humans is responsible for a variant of neuronal ceroid lipofuscinosis (NCL) (Smith et al., supra), a group of monogenic neurodegenerative disease of multiple etiologies. GRN-deficient cells show exacerbated inflammatory reactions, such as enhanced caspase activation, but lower cellular survival, vulnerability to oxygen and glucose deprivation, oxidative stress, kinase and proteasomal inhibitors (e.g. Almeida et al., Cell Rep. 2(4):789-98, 2012; Guo et al., Brain Res. 1366:1-8, 2010; Kleinberger et al., J Neurochem. 115(3):735-47, 2010; and Yin et al., J Exp Med. 207(1):117-28, 2010).

Numerous transgenic mouse models of FTD were generated over the past few years to characterize the underlying pathological and behavioral alterations. Progranulin knockout (Grn−/−) mice harbors neuroinflammation, abnormal social interactions, increased grooming behaviour, fear-avoidance, decreased bone mass and other key FTD features and has been used widely (Martens et al., supra).

Gene therapy approaches to treat Alzheimer's disease (AD) or FTD by introducing a functional progranulin gene capable of restoring progranulin expression has been proposed. For example, Minami et al. (Nature medicine. 20(10) 1157-1164, 2014) demonstrate that lentiviral vectors capable of overexpressing progranulin injected directly into the hippocampus of mice inhibited amyloid β (Aβ) deposition, which is a hallmark of AD.

FTD and amyotrophic lateral sclerosis (ALS) are two related neurodegenerative disorders with overlapping molecular disease pathways, and TAR DNA binding protein 43 (TDP-43) is the main disease protein in most patients with ALS and about 50% of patients with FTD. TDP-43 pathology is not restricted to patients with missense mutations in TARDBP, the gene encoding TDP-43, but also occurs in ALS/FTD patients without known genetic cause or in patients with various other ALS/FTD gene mutations. Mutations in progranulin, which result in a reduction of ˜50% of progranulin protein (PGRN) levels, cause FTD with TDP-43 pathology. Beal et al. (Molecular Neurodegeneration 13:55, 2018) demonstrate that the overexpression of PGRN reduced the levels of insoluble TDP-43 and significantly slowed down disease progression, extending the median survival by approximately 130 days. The study revealed an important role of PGRN in attenuating mutant TDP-43-induced neurodegeneration. A transcriptome analysis did not point towards a single pathway affected by PGRN, but rather towards a pleiotropic effect on different pathways.

Arrant et al. (Brain. 140:1447-1465, 2017) demonstrate that restoration of progranulin with an adeno-associated virus (AAV) vector to GRN^{+/-} mice corrects social behaviour deficits. In GRN^{-/-} mice, Arrant et al., (J of Neuroscience 38(9):2341-2358, 2018), showed that AAV-expressed progranulin was only detected in neurons, not microglia. Further, 8 out of 9 AAV-Grn-treated Grn^{-/-} mice displayed anti-mouse progranulin antibodies, suggesting that AAV-GRN induced a nonself reaction in these mice. In two studies, nonself reactions to AAV transgenes provoked a cell-mediated immune response in which effector T cells killed muscle cells expressing AAV transgene, resulting in loss of transgene expression (Yuasa et al. Gene Ther. 14:1249-1260, 2007; Medell et al. N Engl J Med. 363:1429-1437, 2010).

New approaches for treating neurodegenerative diseases such as FTD, and those mediated by deficiencies in progranulin levels are required because there is currently no cure for FTD and no treatment has been shown to slow progression of the disease.

Haematopoietic cell transplantation (HCT) is a curative therapy for several inherited and acquired disorders, including monogenic central nervous system (CNS) disorders, upon replacement of brain myeloid cells, including microglia, by the transplant-derived cells. However, allogeneic HCT is limited by the poor availability of matched donors, the mortality associated with the allogeneic procedure which is mostly related to graft-versus-host disease (GvHD), and infectious complications provoked by the profound and long-lasting state of immune dysfunction.

Gene therapy approaches based on the transplantation of genetically modified autologous HSCs offer potentially improved safety (and efficacy, upon improved/enhanced expression of therapeutic genes) over allogeneic HCT. They are particularly relevant for patients lacking a matched donor.

Thus, in monogenic neurodegenerative disorders, HSCGT based on the use of lentiviral vector (LV) mediated gene transferred or gene edited HSPCs can be therapeutic and offers the benefit of long-lasting delivery of the therapeutic proteins to the CNS by the transplant derived CNS progeny cells. The intra-CNS delivery of HSPCs is expected to increase the specificity and therapeutic potential of this approach for chronic neurodegenerative diseases such as FTD.

### SUMMARY OF THE INVENTION

The invention relates to the field of gene therapy and more specifically to hematopoietic stem and progenitor cells that have been genetically modified to express functional progranulin. Suitably, HSPCs that have been gene edited using, for example, CRISPR/Cas technology for targeted gene addition or have been transduced with viral gene delivery vehicles, such as lentiviral vectors (LV). The invention also relates to the use of such modified HSPCs in therapy. In particular, the invention relates to use of the modified progranulin expressing HSPCs in the prevention or treatment of a neurodegenerative disease mediated by dysfunctional progranulin expression. In particular, the invention relates to haematopoietic stem and progenitor cells capable of expressing progranulin due to introduction into the HSPC of nucleic acid encoding progranulin via a viral vector, such as LV, or targeted gene addition by CRISPR/Cas technology. Optionally, the HSPC has also been modified to express metallothionein, such as via metallothionein 1G gene. The invention further comprises a method for treating neurodegenerative diseases mediated by enhancing progranulin expression above endogenous levels. The invention thus embraces the treatment of neurodegenerative diseases such as frontotemporal dementia (FTD), neuronal ceroid lipofuscinosis (NCL), amyotrophic lateral sclerosis (ALS), Alzheimer's disease (AD) and Parkinson's disease (PD) by introducing into the patient (e.g., a mammalian patient, such as a human patient (e.g., an adult human patient)) in need thereof HSPCs modified to express progranulin.

According to a first aspect of the invention there is provided a population of modified hematopoietic stem and progenitor cells (HSPCs) where the cells carry an exogenous copy of a nucleic acid encoding progranulin. Suitably the progranulin is human progranulin. Optionally, the HSPCs also carry an exogenous copy of a nucleic acid encoding a metallothionein, such as human metallothionein 1G. Cells that carry an exogenous copy of a nucleic acid may carry more than one copy, e.g. 2, 3, 4, 5, 6, 7, 8, 9, 10 or more copies of the nucleic acid encoding the progranulin or metallothionein 1G.

Suitably the method is carried out in vitro or ex vivo. Optionally, the HSPC also carries one or more exogenous copies of a nucleic acid encoding a metallothionein, such as human metallothionein 1G.

In particular embodiments the nucleic acid that encodes progranulin (and optionally metallothionein) has been introduced by gene editing, such as CRISPR/cas, TALENs or Zinc finger methodology.

In other embodiments the nucleic acid that encodes progranulin (and optionally metallothionein) has been introduced by viral vector transduction. E.g. using a lentiviral, adenoviral, adeno-associated or avian virus system.

According to a second aspect of the invention there is provided a method for producing a population of HSPCs modified to express progranulin, the method comprising contacting a sample of HSPCs with a vector carrying an exogenous copy of a nucleic acid encoding progranulin under conditions to allow the vector to transfect or transduce the HSPCs.

Suitably the method is carried out in vitro or ex vivo. Optionally, the same or a separate vector carries exogenous copies of a nucleic acid encoding a metallothionein, such as human metallothionein 1G. Suitably the vector is a lentiviral vector.

According to a third aspect of the invention there is provided a composition comprising the HSPCs of the first aspect of the invention or the HSPC produced by the second aspect of the invention.

According to a further aspect of the invention there is provided the HSPCs of the first aspect of the invention or the HSPC produced by the second aspect of the invention or a composition of the third aspect of the invention for use in therapy.

According to a fourth aspect of the invention there is provided a method of treating a neurodegenerative disorder mediated by aberrant expression of progranulin, such as frontotemporal dementia (FTD) or neuronal ceroid lipofuscinosis (NCL), in a subject in need thereof, the method comprising administering to the subject the HSPCs of the first aspect of the invention or the HSPC produced by the second aspect of the invention or a composition of the third aspect of the invention.

According to a fifth aspect of the invention there is provided a population of progranulin expressing HSPCs of the first aspect of the invention or the HSPCs produced by the second aspect of the invention or the composition of the third aspect of the invention for use in the prevention or treatment of a neurodegenerative disorder mediated by aberrant expression of progranulin such as frontotemporal dementia (FTD) or neuronal ceroid lipofuscinosis (NCL).

According to a sixth aspect of the invention there is provided a method of preventing or treating a disease mediated by a dysfunctional gene encoding progranulin, the method comprising:
(i) determining whether a subject has a mutation in the progranulin gene that leads to aberrant expression or amount of progranulin; and,
(ii) if the subject has a mutation in the progranulin gene that leads to aberrant expression or amount of progranulin they are administered the HSPCs of the first aspect of the invention or the HSPC produced by the second aspect if the invention or a composition of the third aspect of the invention.

According to a variant of the sixth aspect of the invention there is provided a population of modified HSPCs of the first aspect of the invention or the HSPC produced by the second aspect if the invention or a composition of the third aspect of the invention for use in the prevention or treatment of a disease mediated by a dysfunctional gene encoding progranulin, the method comprising:
(i) determining whether a subject has a mutation in the progranulin gene that leads to aberrant expression or amount of progranulin; and,
(ii) if the subject has a mutation in the progranulin gene that leads to aberrant expression or amount of progranulin they are administered the population of modified HSPCs.

In a particular embodiment, the modified HSPCs are administered by intracerebroventricular (ICV) and/or intrathecal lumbar (ITL) and/or intravenous (IV) injection.

In embodiments of the fourth, fifth or sixth aspects the neurodegenerative disorder mediated by aberrant expression of progranulin is frontotemporal dementia (FTD).

In some embodiments of the above aspects, the HSPC is CD34⁺, Lin⁻, CD38⁻, and/or CD90⁺. In a particular embodiment of the above aspects the HSPC is CD34⁺. In various embodiments of the above aspects, the HSPC is functionally equivalent to a myeloid/microglia progenitor cell upon transplantation. In various embodiments of the above aspects, the HSPC engrafts in the brain.

In various embodiments of the above aspects, the engrafted HSPC is functionally equivalent to a microglia progenitor cell as it generates a microglia-like progeny. In various embodiments of the above aspects, the subject undergoes ablative conditioning prior to the method. In various embodiments of the above aspects, the ablative conditioning comprises administering to the subject an alkylating agent. In various embodiments of the above aspects, the alkylating agent is busulfan. In various embodiments of the above aspects, the HSPC is an allogeneic or autologous cell.

Other features and advantages of the invention will be apparent from the detailed description, and from the claims.

### DESCRIPTION OF THE DRAWINGS

FIG. 1 shows a representative immunoblot comparing the relative level of expression of human progranulin in control untransduced GRN^{-/-} (knockout) HAP1 cells, control wild type HAP1 cells, and GRN^{-/-} HAP1 cells transduced with lentivirus encoding for the human GRN (wild-type or codon optimized) at different multiplicity of infection (MOI). The immunoblot was probed with an anti-human GRN antibody, stripped and re-probed with an anti-GAPDH antibody to show relative loadings. Western blot analysis was performed by loading 25 μg of total protein from whole cell lysate. UT=Un-transduced cells; WT=Wild type cells; MOI=Multiplicity of infection; Lad=Ladder.

FIG. 2: hGRN_wt is more expressed that hGRN_co in Hela and HAP1 genetically corrected GRN^{-/-} cell lines.

Human GRN^{-/-} Hela or HAP1 cells were transduced with the hGRN_wt or hGRN_co LVs and single cell clones generated in order to compare hGRN expression at a specific VCN. Clones with VCN 1, 2 and 3 were selected (n=2 for each LV) and WBs performed on total cell lysates probing with an anti-human GRN specific antibody. A) Representative immunoblot showing hGRN expression in Hela clones transduced with the hGRN_wt or hGRN_co LV. The immunoblots show the hGRN specific band in parental (wild-type GRN^{+/+}) and transduced cells, while no band is detected in control untransduced GRN^{-/-} cells. GAPDH (37 kDa) was used as internal loading control. B) Densitometry analysis of WB comparing hGRN expression between hGRN_wt and hGRN_co transduced clones. hGRN expression was normalized to GAPDH (loading control) and to the level of hGRN expression in parental Hela cells. C) Representative immunoblot showing hGRN expression in HAP1 transduced clones. D) Densitometry analysis of WB. hGRN expression was normalized to GAPDH (loading control) and to the level of hGRN expression in parental HAP1 cells. Data are presented as mean±SD, n=2 technical replicates from 2 biological replicates. Parental=Control wild-type GRN^{+/+} cells; UT=Control untransduced GRN^{-/-} cells cells; WT=Cells transduced with hGRN_wt LV; CO=Cells transduced with hGRN_co LV.

FIG. 3: hGRN_wt advantage at release and cross-correction of GRN^{-/-} Hela and HAP1 cells

A) Schematic diagram showing the experimental procedure to evaluate the capacity of GRN-LV transduced cells to cross-correct GRN^{-/-} target cells. hGRN_wt or hGRN_co LV transduced (producing) cells (Hela or HAP1) are cultured at cellular confluence for 48-72 hours. Cell culture supernatant is collected at 48 and 72 hours of culture and used to measure hGRN content and treat GRN^{-/-} target cells. Following 24 and/or 48 hours of conditioning treatment, target cells are collected to evaluate hGRN internalization by ELISA assay. (B) hGRN concentration (ng/ml) in the medium of LV-transduced and control GRN^{-/-} untransduced (UT) Hela cells. C) Graph showing the amount of hGRN detected in GRN^{-/-} Hela cells following treatment (24 or 48 hours) with conditioned medium from LV-transduced (producing) cells. D) hGRN concentration (ng/ml) in the medium of LV-transduced and control GRN^{-/-} untransduced (UT) HAP1 cells. E) hGRN concentration in GRN^{-/-} HAP1 cells following treatment with conditioned medium from LV-transduced (producing) cells. Data are presented as mean±SD, n=2 separate experiments (*p<0.05, 1-way ANOVA).

FIG. 4: IFNγ stimulation of MHC3 induces TSPO and MHC-II expression HMC3 cells were transduced with hGRN encoding LVs in which the expression of the transgene is driven by i) the strong and constitutive hPGK promoter (hPGK-GRN); ii) the microglia/myeloid regulated promoter based on the minimally regulatory elements of the human HLA-DRA promoter (hHLA-GRN); or iii) the inducible promoter derived from the human TSPO (hTSPO-GRN). Mock-transduced (UT) and LV-transduced HMC3 were activated by incubation with IFNγ 100 ng/ml for 72 hours. Mock-activated cells were maintained in the same culture conditions without IFNγ. A) As a positive marker for cell activation, the cell surface expression level of Major Histocompatibility Complex (MHC) class-II molecule was evaluated by flow cytometry (FC) analysis at the end of the induction protocol. B) The expression level for the endogenous human HLA-DRA was evaluated by duplex TaqMan real-time PCR assays in mock-activated (resting) and IFNγ activated HMC3 cells. The human Glyceraldehyde 3-Phosphate Dehydrogenase (GAPDH) was used as a reference gene (N=2 experiments, run in duplicate). B) Transcriptional level of the endogenous TSPO in mock-activated (resting) and IFN-activated HMC3 cells.

FIG. 5: IFNγ stimulation of hHLA-GRN LV-transduced MHC3 cells induces hGRN gene overexpression

Total RNA was extracted from mock-transduced and LV-transduced HMC3 cells activated or not with the INF-γ, reverse-transcribed into cDNA and used as a template for downstream gene expression analysis. Duplex TaqMan real-time PCR assay specific for the LV transcript (WPRE), coupled to the human Glyceraldehyde 3-Phosphate Dehydrogenase (GAPDH) as a reference gene (N=2 experiments, run in duplicate) was performed to evaluate the responsiveness to cell-activation of the synthetic hTSPO and hHLA LV promoters. (A) Lentiviral transcription (WPRE) in mock-transduced cells and IFN-activated HMC3 cells. (D) WPRE fold increase in mock transduced (UT) and LV transduced HMC3 cells upon IFN-γ cell-activation.

FIG. 6: IFNγ stimulation of hHLA-GRN LV-transduced MHC3 cells induces hGRN protein overexpression

hGRN protein expression (WB) and release (ELISA) was evaluated in mock-transduced and LV-transduced HMC3 cells before and after IFNγ stimulation. (A) Representative immunoblot showing GRN expression in UT and LV-transduced HMC3 cells before and after cell-activation. (B) Densitometry analysis of western blot results. GRN expression was normalized to GAPDH (loading control) and to the basal level of expression in UT HMC3 cells. C) Supernatant from mock-transduced and LV-transduced HMC3 cells was collected and hGRN quantified by ELISA assay. hGRN secretion (ng/ml) is compared between non-induced and IFNγ induced HMC3 cells.

FIG. 7: wt Lin⁻ HSPCs are efficiently transduced with GRN-encoding LVs

Lin⁻ HSPCs were obtained from wt C57BL/6J donor mice and transduced with hPGK-GRN, hTSPO-GRN or hHLA-GRN LVs at different MOIs. A) Summary table showing LV transduction efficiency (measured as VCN) in Lin⁻ cells with the GRN LVs at different MOIs. B) hGRN secretion (pg/ml) from mock-transduced (UT) and LV-transduced 12 days liquid cultures as measured by ELISA assay on cell media. Data was normalized to the number of cells at the end of the culture and to VCN.

FIG. 8: ICV HSPC transplantation in FTD mice: Brain engraftment and hGRN

A) Brain from mice receiving hPGK-GFP transduced HSPCs was processed for flow cytometry analysis to assess myeloid cell reconstitution after ICV injection (% GFP⁺ in Cd45⁺ Cd11b⁺ total myeloid brain compartment). B) hGRN concentration in brain protein extracts of engrafted mice as measured by ELISA assay. hGRN was normalized on total protein content and VCN/engraftment.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The following definitions may be useful in the understanding of the invention.

As used herein, the terms “ablate,” “ablating,” “ablation,” “condition,” “conditioning,” and the like refer to the depletion of one or more cells in a population of cells in vivo or ex vivo. In some embodiments of the present disclosure, it may be desirable to ablate endogenous cells within a patient (e.g., a patient undergoing treatment for a disease described herein) before administering a therapeutic composition, such as a therapeutic population of cells, to the patient. This can be beneficial, for example, in order to provide newly-administered cells with an environment within which the cells may engraft. Ablation of a population of endogenous cells can be performed in a manner that selectively targets a specific cell type, for example, using antibodies or antibody-drug conjugates that bind to an antigen expressed on the target cell and subsequently engender the killing of the target cell. Additionally, or alternatively, ablation may be performed in a non-specific manner using cytotoxins that do not localize to a particular cell type but are instead capable of exerting their cytotoxic effects on a variety of different cells. Examples of ablation include depletion of at least 5% of cells (e.g., at least 5%, 10%, 15%, 20%, 25%, 30%, 35%, 40%, 45%, 50%, or more) in a population of cells in vivo or in vitro. Quantifying cell counts within a sample of cells can be performed using a variety of cell-counting techniques, such as through the use of a counting chamber, a Coulter counter, flow cytometry, or other cell-counting methods known in the art.

Exemplary agents that can be used to “ablate” a population of cells in a patient (i.e., to “condition”) a patient for treatment) in accordance with the compositions and methods of the disclosure include alkylating agents, such as nitrogen mustards (e.g., bendamustine, chlorambucil, cyclophosphamide, ifosfamide, mechlorethamine, or melphalan), nitrosoureas (e.g., carmustine, lomustine, or streptozocin), alkyl sulfonates (e.g., busulfan), triazines (e.g., dacarbazine or temozolomide), or ethylenimines (e.g., altretamine or thiotepa). In some embodiments, the one or more conditioning agents are non-myeloablative conditioning agents that selectively target and ablate a specific population of endogenous pluripotent cells, such as a population of endogenous CD34+HSCs or HPCs. For example, the one or more conditioning agents may include cytarabine, antithymocyte globulin, fludarabine, or idarubicin. Ablation can also be performed using radiation or chemotherapeutic agents.

As used herein, the term “about” refers to a quantity that varies by as much as 30% (e.g., 25%, 20%, 25%, 10%, 9%, 8%, 7%, 6%, 5%, 4%, 3%, 2% or 1%) relative to a reference quantity.

As used herein in the context of a protein of interest, the term “activity” refers to the biological functionality that is associated with a wild-type form of the protein. For example, in the context of an enzyme, the term “activity” refers to the ability of the protein to effectuate substrate turnover in a manner that yields the product of a corresponding chemical reaction. Activity levels of enzymes can be detected and quantitated, for example, using substrate turnover assays known in the art. As another example, in the context of a membrane-bound receptor, the term “activity” may refer to signal transduction initiated by the receptor, e.g., upon binding to its cognate ligand. Activity levels of receptors involved in signal transduction pathways can be detected and quantitated, for example, by observing an increase in the outcome of receptor signalling, such as an increase in the transcription of one or more genes (which may be detected, e.g., using polymerase chain reaction techniques known in the art).

As used herein, the terms “administering,” “administration,” and the like refer to directly giving a patient a therapeutic agent (e.g., a population of modified HSPCs of the invention) by any effective route. Exemplary routes of administration are described herein and include systemic administration routes, such as intravenous (IV), intracerebroventricular (ICV), intra-cisterna magna (ICM) or lumber intrathecal (IT) injection, among others.

As used herein, the term “allogeneic” refers to cells, tissues, nucleic acid molecules, or other substances obtained or derived from a different subject of the same species. For example, in the context of a population of cells (e.g., a population of pluripotent cells) expressing one or more proteins described herein, allogeneic cells include those that are (i) obtained from a subject that is not undergoing therapy and are then (ii) transduced or transfected with a vector that directs the expression of one or more desired proteins (e.g. progranulin). The phrase “directs expression” refers to the inclusion of one or more polynucleotides encoding the one or more proteins to be expressed. The polynucleotide may contain additional sequence motifs that enhances expression of the protein of interest.

As used herein, the term “autologous” refers to cells, tissues, nucleic acid molecules, or other substances obtained or derived from an individual's own cells, tissues, nucleic acid molecules, or the like. For example, in the context of a population of cells (e.g., a population of pluripotent cells) expressing one or more proteins described herein, autologous cells include those that are obtained from the patient undergoing therapy that are then transduced or transfected with a vector that directs the expression of one or more proteins of interest.

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15 (20): 8125-8148, incorporated herein by reference in its entirety. Multiple stop codons can be incorporated.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell) or one that has been added to a cell, thus in the context of the present invention, the exogenous copy of a nucleic acid encoding progranulin is one that has been introduced to the cell or a parent precursor cell that the cell has arisen from (e.g. a microglia cell from a modified HSPC). Exogenous materials include those that are provided from an external source to an organism or to cultured matter extracted there from.

As used herein, the term “expansion agent” refers to a substance capable of promoting the proliferation of a given cell type ex vivo. Accordingly, a “hematopoietic stem cell expansion agent” or an “HSC expansion agent” refers to a substance capable of promoting the proliferation of a population of hematopoietic stem cells ex vivo. Hematopoietic stem cell expansion agents include those that effectuate the proliferation of a population of hematopoietic stem cells such that the cells retain hematopoietic stem cell functional potential. Exemplary hematopoietic stem cell expansion agents that may be used in conjunction with the compositions and methods of the disclosure include, without limitation, aryl hydrocarbon receptor antagonists, such as those described in U.S. Pat. Nos. 8,927,281 and 9,580,426, the disclosures of each of which are incorporated herein by reference in their entirety, and, in particular, compound SR1. Additional hematopoietic stem cell expansion agents that may be used in conjunction with the compositions and methods of the disclosure include compound UM-171 and other compounds described in U.S. Pat. No. 9,409,906, the disclosure of which is incorporated herein by reference in its entirety. Hematopoietic stem cell expansion agents further include structural and/or stereoisomeric variants of compound UM-171, such as the compounds described in US 2017/0037047, the disclosure of which is incorporated herein by reference in its entirety. Additional hematopoietic stem cell expansion agents suitable for use in the instant disclosure include histone deacetylase (HDAC) inhibitors, such as trichostatin A, trapoxin, trapoxin A, chlamydocin, sodium butyrate, dimethyl sulfoxide, suberanilohydroxamic acid, m-carboxycinnamic acid bishydroxamide, HC-toxin, Cyl-2, WF-3161, depudecin, and radicicol, among others described, for example, in WO 2000/023567, the disclosure of which is incorporated herein by reference.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a subject can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the subject. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the term “Frontotemporal dementia” (FTD) refers to the neurodegenerative condition which is the second most common cause of dementia in people under the age of 65 years. A fraction of FTD patients have autosomal dominant mutations in GRN gene.

As used herein, the term “functional potential” as it pertains to a pluripotent cell, such as a hematopoietic stem cell, refers to the functional properties of stem cells which include: 1) multi-potency (which refers to the ability to differentiate into multiple different blood lineages including, but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells); 2) self-renewal (which refers to the ability of stem cells to give rise to daughter cells that have equivalent potential as the mother cell, and further that this ability can repeatedly occur throughout the lifetime of an individual without exhaustion); and 3) the ability of stem cells or progeny thereof to be reintroduced into a transplant recipient whereupon they home to the stem cell niche and re-establish productive and sustained cell growth and differentiation.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34⁺ cells. CD34+ cells are immature cells that express the CD34 cell surface marker. In humans, CD34⁺ cells are believed to include a subpopulation of cells with the stem cell properties defined above, whereas in mice, HSCs are CD34⁻. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC can be CD34⁺, CD38⁻, CD45RA⁻, CD90⁺, CD49F⁺, and lin⁻ (negative for mature lineage markers including CD2, CD3, CD4, CD7, CD8, CD10, CD11B, CD19, CD20, CD56, CD235A). In mice, bone marrow LT-HSC can be CD34⁺, SCA-1⁺, C-kit⁺, CD135⁻, Slamf1/CD150⁺, CD48⁻, and lin⁻ (negative for mature lineage markers including Ter119, CD11b, Gr1, CD3, CD4, CD8, B220, IL-7ra), whereas ST-HSC can be CD34⁺, SCA-1⁺, C-kit⁺, CD135⁻, Slamf1/CD150⁺, and lin⁻ (negative for mature lineage markers including Ter119, CD11b, Gr1, CD3, CD4, CD8, B220, IL-7ra). In addition, ST-HSC are less quiescent (i.e., more active) and more proliferative than L T-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny. Hematopoietic progenitor cells are descendants of HSCs that are capable of further differentiation to become specialised cells. Stem cells that self-renew produce more stem cells, those that start down the path of differentiation produce progenitor cells. Thus, when it comes to cell differentiation, HPCs fall on the spectrum between hematopoietic stem cells and fully differentiated (mature) cells.

Typically, HSCs and HPCs are isolated from bone marrow or peripheral blood together.

A population comprising HSCs and/or HPCs are referred to herein as HSPCs.

As used herein, an agent that inhibits histone deacetylation refers to a substance or composition (e.g., a small molecule, protein, interfering RNA, messenger RNA, or other natural or synthetic compound, or a composition such as a virus or other material composed of multiple substances) capable of attenuating or preventing the activity of histone deacetylase, more particularly its enzymatic activity either via direct interaction or via indirect means such as by causing a reduction in the quantity of a histone deacetylase produced in a cell or by inhibition of the interaction between a histone deacetylase and an acetylated histone substrate. Inhibiting histone deacetylase enzymatic activity means reducing the ability of a histone deacetylase to catalyze the removal of an acetyl group from a histone residue (e.g., a mono-, di-, or tri-methylated lysine residue; a monomethylated arginine residue, or a symmetric/asymmetric dimethylated arginine residue, within a histone protein). Preferably, such inhibition is specific, such that the agent that inhibits histone deacetylation reduces the ability of a histone deacetylase to remove an acetyl group from a histone residue at a concentration that is lower than the concentration of the inhibitor that is required to produce another, unrelated biological effect.

As used herein, the terms “histone deacetylase” and “HDAC” refer to any one of a family of enzymes that catalyze the removal of acetyl groups from the ε-amino groups of lysine residues at the N-terminus of a histone. Unless otherwise indicated by context, the term “histone” is meant to refer to any histone protein, including HI, H2A, H2B, H3, H4, and H5, from any species. Human HDAC proteins or gene products, include, but are not limited to, HDAC-1, HDAC-2, HDAC-3, HDAC-4, HDAC-5, HDAC-6, HDAC-7, HDAC-8, HDAC-9, HDAC-10, and HDAC-11.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and, are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-pluripotent cell that can include, for example, Oct3/4, Sox family transcription factors (e.g., Sox1, Sox2, Sox3, Soxl5), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. Human iPS cells are described, for example, in Takahashi and Yamanaka, Cell 126:663 (2006), the disclosure of which is incorporated herein by reference as it pertains to the structure and functionality of iPS cells.

As used herein, the term “inhibitor” refers to an agent (e.g., a small molecule, peptide fragment, protein, antibody, or antigen-binding fragment thereof) that binds to, and/or otherwise suppresses the activity of, a target molecule.

By “metallothionein IG (MTIG) polypeptide” is meant a protein having at least about 85% amino acid sequence identity to UniProt Accession No. P13640-1 or a fragment thereof and having heavy metal binding activity. An exemplary MTIG polypeptide sequence is provided below:

(SEQ ID NO: 3)
MDPNCSCAAAGVSCTCASSCKCKECKCTSCKK
SCCSCCPVGCAKCAQGCICKGASEKCSCCA

By “metallothionein IG (MTIG) polynucleotide” is meant a nucleic acid molecule encoding an MTIG polypeptide. The MTIG gene encodes a protein that binds heavy metals.

An exemplary human MTIG polynucleotide sequence (accession number NM_005950.2 is Homo sapiens metallothionein IG (MTIG), transcript variant 1, mRNA) is provided below:

(SEQ ID NO: 4)
ACTCCGCCTTCCACGTGCACCCACTGCCTCTTCCC
TTCTCGCTTGGGAACTCTAGTCTCGCCTCGGGTTG
CAATGGACCCCAACTGCTCCTGTGCCGCTGGTGTC
TCCTGCACCTGCGCCAGCTCCTGCAAGTGCAAAGA
GTGCAAATGCACCTCCTGCAAGAAGAGCTGCTGCT
CCTGCTGCCCTGTGGGCTGTGCCAAGTGTGCCCAG
GGCTGCATCTGCAAAGGGGCATCGGAGAAGTGCAG
CTGCTGCGCCTGATGTCGGGACAGCCCTGCTCCCA
AGTACAAATAGAGTGACCCGTAAAATCCAGGATTT
TTTGTTTTTTGCTACAATCTTGACCCCTTTGCTAC
ATTCCTTTTTTTCTGTGAAATATGTGAATAATAAT
TAAACACTTAGACTTGAAAAAAAAAAAAAAAAAAA

By “microglia” is meant an immune cell of the central nervous system.

As used herein in the context of hematopoietic stem and/or progenitor cells, the term “mobilization” refers to release of such cells from a stem cell niche where the cells typically reside (e.g., the bone marrow) into peripheral circulation. “Mobilization agents” are agents that are capable of inducing the release of hematopoietic stem and/or progenitor cells from a stem cell niche into peripheral circulation.

As used herein, the term “myeloablative” or “myeloablation” refers to a conditioning regiment that substantially impairs or destroys the hematopoietic system, typically by exposure to a cytotoxic agent or radiation. Myeloablation encompasses complete myeloablation brought on by high doses of cytotoxic agent or total body irradiation that destroys the hematopoietic system. As used herein, the term “non-myeloablative” or “myelosuppressive” refers to a conditioning regiment that does not eliminate substantially all hematopoietic cells of host origin.

As used herein, “neurodegenerative disease” refers to any of a group of diseases characterized by the progressive loss of structure and/or function of neurons, including death of neurons. Exemplary neurodegenerative diseases include, without limitation, neuronal ceroid lipofuscinosis (NCL), amyotrophic lateral sclerosis (ALS), Alzheimer's disease (AD) and Parkinson's disease (PD).

“Neuronal ceroid lipofuscinosis” (NCL) is a group of disorders that affect the nervous system and typically cause progressive problems with vision, movement, and thinking ability. The different NCLs are distinguished by their genetic cause. Each disease type is given the designation “CLN,” meaning ceroid lipofuscinosis, neuronal, and then a number to indicate its subtype. CLN11 results from mutations in GRN gene.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence but, will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or about 250 mM NaCl and 25 mM trisodium citrate. Low stringency hybridization can be obtained in the absence of organic solvent, e.g., formamide, while high stringency hybridization can be obtained in the presence of at least about 35% formamide, and in some embodiments, at least about 50% formamide. Stringent temperature conditions will ordinarily include temperatures of at least about 30° C. at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature.

As above, wash stringency can be increased by decreasing salt concentration or by increasing temperature. For example, stringent salt concentration for the wash steps will comprise less than about 30 mM NaCl and 3 mM trisodium citrate or less than about 15 mM NaCl and 1.5 mM trisodium citrate. Stringent temperature conditions for the wash steps will ordinarily include a temperature of at least about 25° C., at least about 42° C., or at least about 68° C. In some embodiments, wash steps will occur at 25° C. in 30 mM NaCl, 3 mM trisodium citrate, and 0.1% SDS. In other embodiments, wash steps will occur at 42 C in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by(the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “pharmaceutical composition” refers to a composition containing a therapeutic agent (e.g., modified HSPCs of the invention) that may be administered to a subject, such as a mammal, e.g., a human, in order to prevent, treat or control a particular disease or condition affecting the mammal, such as FTD as described herein.

As used herein, the term “pluripotent cell” refers to a cell that possesses the ability to develop into more than one differentiated cell type, such as a cell type of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of pluripotent cells are ESCs, iPSCs, and CD34⁺ cells.

“Progranulin” is a secreted highly glycosylated protein that acts as a key regulator of lysosornal function and as a growth factor involved in inflammation, wound healing, early embryonic development and cell proliferation. It regulates protein trafficking to lysosomes and, also the activity of lysosomal enzymes (see Babykumari, et al., BRAIN. 140:3081-3104, 2017). Progranulin is a 593-amino acid, cysteine rich protein with an estimated molecular weight of 68.5 kDa and it is encoded by the PGRN gene on chromosome 17q21. The protein is also known as epithelin precursor, prepithelin and prostate cancer cell derived growth factor. Progranulin is composed by seven and a half tandem repeats of a 12-cysteine module called granulin-like domains. Within lysosomes and upon its release, progranulin can be cleaved by extracellular proteases, such as elastase, into smaller individual granulin peptides ranging from 6 to 25 kDa is size. Although granulins possess biological functions, most of the research has been conducted on the full length progranulin protein which has a much stronger molar potency compared to individual granulins. Progranulin is a ubiquitously expressed protein mostly found in the lysosome membranes of epithelial and hematopoietic cells. In the central nervous system progranulin is mainly produced by neurons and activated microglia.

Although little is known about the physiological role of progranulin in the brain, compelling evidence suggests that it is critical to the survival of neuronal cells, neurite outgrowth and in regulating neuro-inflammation in response acute or chronic brain injury. In particular, progranulin possesses strong anti-inflammatory activity and potentially brain repair properties. In the literature, the acronyms PGRN and GRN are often used interchangeably to describe the protein with Gen Bank accession number NP_002078.1 or UniProt reference sequence P28799-1. Herein, the acronyms PGRN and GRN are used interchangeably to refer to a progranulin protein or encoding nucleic acid, as the context dictates.

SEQ ID NO: 2 corresponds to UniProt reference sequence P28799-1 is the amino acid sequence of isoform 1 of progranulin (the canonical sequence), and is shown below:

(SEQ ID NO: 2)
MWTLVSWVALTAGLVAGTRCPDGQFCPVACCLDPG
GASYSCCRPLLDKWPTTLSRHLGGPCQVDAHCSAG
HSCIFTVSGTSSCCPFPEAVACGDGHHCCPRGFHC
SADGRSCFQRSGNNSVGAIQCPDSQFECPDFSTCC
VMVDGSWGCCPMPQASCCEDRVHCCPHGAFCDLVH
TRCITPTGTHPLAKKLPAQRTNRAVALSSSVMCPD
ARSRCPDGSTCCELPSGKYGCCPMPNATCCSDHLH
CCPQDTVCDLIQSKCLSKENATTDLLTKLPAHTVG
DVKCDMEVSCPDGYTCCRLQSGAWGCCPFTQAVCC
EDHIHCCPAGFTCDTQKGTCEQGPHQVPWMEKAPA
HLSLPDPQALKRDVPCDNVSSCPSSDTCCQLTSGE
WGCCPIPEAVCCSDHQHCCPQGYTCVAEGQCQRGS
EIVAGLEKMPARRASLSHPRDIGCDQHTSCPVGQT
CCPSLGGSWACCQLPHAVCCEDRQHCCPAGYTCNV
KARSCEKEVVSAQPATFLARSPHVGVKDVECGEGH
FCHDNQTCCRDNRQGWACCPYRQGVCCADRRHCCP
AGFRCAARGTKCLRREAPRWDAPLRDPALRQLL.

Various other isoforms have been identified for human progranulin. In isoform 2 (identifier P28799-2) positions 377-531 are missing. In isoform 3 (identifier: P28799-3) 1-71 is altered and 72-251 is missing.

An exemplary progranulin nucleic acid sequence, which corresponds to SEQ ID NO:1 (accession number NM_002087.4 in Homo sapiens granulin precursor (GRN), mRNA) is provided below:

(SEQ ID NO: 1)
ATGTGGACCCTGGTGAGCTGGGTGGCCTTAACAGC
AGGGCTGGTGGCTGGAACGCGGTGCCCAGATGGTC
AGTTCTGCCCTGTGGCCTGCTGCCTGGACCCCGGA
GGAGCCAGCTACAGCTGCTGCCGTCCCCTTCTGGA
CAAATGGCCCACAACACTGAGCAGGCATCTGGGTG
GCCCCTGCCAGGTTGATGCCCACTGCTCTGCCGGC
CACTCCTGCATCTTTACCGTCTCAGGGACTTCCAG
TTGCTGCCCCTTCCCAGAGGCCGTGGCATGCGGGG
ATGGCCATCACTGCTGCCCACGGGGCTTCCACTGC
AGTGCAGACGGGCGATCCTGCTTCCAAAGATCAGG
TAACAACTCCGTGGGTGCCATCCAGTGCCCTGATA
GTCAGTTCGAATGCCCGGACTTCTCCACGTGCTGT
GTTATGGTCGATGGCTCCTGGGGGTGCTGCCCCAT
GCCCCAGGCTTCCTGCTGTGAAGACAGGGTGCACT
GCTGTCCGCACGGTGCCTTCTGCGACCTGGTTCAC
ACCCGCTGCATCACACCCACGGGCACCCACCCCCT
GGCAAAGAAGCTCCCTGCCCAGAGGACTAACAGGG
CAGTGGCCTTGTCCAGCTCGGTCATGTGTCCGGAC
GCACGGTCCCGGTGCCCTGATGGTTCTACCTGCTG
TGAGCTGCCCAGTGGGAAGTATGGCTGCTGCCCAA
TGCCCAACGCCACCTGCTGCTCCGATCACCTGCAC
TGCTGCCCCCAAGACACTGTGTGTGACCTGATCCA
GAGTAAGTGCCTCTCCAAGGAGAACGCTACCACGG
ACCTCCTCACTAAGCTGCCTGCGCACACAGTGGGG
GATGTGAAATGTGACATGGAGGTGAGCTGCCCAGA
TGGCTATACCTGCTGCCGTCTACAGTCGGGGGCCT
GGGGCTGCTGCCCTTTTACCCAGGCTGTGTGCTGT
GAGGACCACATACACTGCTGTCCCGCGGGGTTTAC
GTGTGACACGCAGAAGGGTACCTGTGAACAGGGGC
CCCACCAGGTGCCCTGGATGGAGAAGGCCCCAGCT
CACCTCAGCCTGCCAGACCCACAAGCCTTGAAGAG
AGATGTCCCCTGTGATAATGTCAGCAGCTGTCCCT
CCTCCGATACCTGCTGCCAACTCACGTCTGGGGAG
TGGGGCTGCTGTCCAATCCCAGAGGCTGTCTGCTG
CTCGGACCACCAGCACTGCTGCCCCCAGGGCTACA
CGTGTGTAGCTGAGGGGCAGTGTCAGCGAGGAAGC
GAGATCGTGGCTGGACTGGAGAAGATGCCTGCCCG
CCGGGCTTCCTTATCCCACCCCAGAGACATCGGCT
GTGACCAGCACACCAGCTGCCCGGTGGGGCAGACC
TGCTGCCCGAGCCTGGGTGGGAGCTGGGCCTGCTG
CCAGTTGCCCCATGCTGTGTGCTGCGAGGATCGCC
AGCACTGCTGCCCGGCTGGCTACACCTGCAACGTG
AAGGCTCGATCCTGCGAGAAGGAAGTGGTCTCTGC
CCAGCCTGCCACCTTCCTGGCCCGTAGCCCTCACG
TGGGTGTGAAGGACGTGGAGTGTGGGGAAGGACAC
TTCTGCCATGATAACCAGACCTGCTGCCGAGACAA
CCGACAGGGCTGGGCCTGCTGTCCCTACCGCCAGG
GCGTCTGTTGTGCTGATCGGCGCCACTGCTGTCCT
GCTGGCTTCCGCTGCGCAGCCAGGGGTACCAAGTG
TTTGCGCAGGGAGGCCCCGCGCTGGGACGCCCCTT
TGAGGGACCCAGCCTTGAGACAGCTGCTGTGA

An exemplary murine progranulin nucleic acid sequence is disclosed in SEQ ID NO: 5.

An exemplary codon-optimised human progranulin nucleic acid sequence has the sequence in SEQ ID NO: 6.

As used herein, the term “functional progranulin” refers to a wild-type form of the progranulin gene or protein, as well as variants (e.g., isoforms, splice variants, truncations, concatemers, and fusion constructs, among others) of wild-type progranulin proteins and nucleic acids encoding the same, so long as such variants retain normal, physiological abilities of wild-type progranulin, such as the ability of being secreted following post-translational modifications, to regulate lysosomal abundance and activity, to promote neuronal survival, and to function as anti-inflammatory molecule. Examples of such variants may include proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type progranulin protein (e.g., SEQ ID NO: 2), such as variants having an amino acid sequence that differs from that of wild-type progranulin by way of one or more conservative amino acid substitutions, provided that the progranulin variant retains the therapeutic function of a wild-type progranulin. Suitably, the expressed functional progranulin will have approximately the same activity as wild-type progranulin in equivalent healthy cells, though modified cells that express greater or lesser activity, for example one with 20%-200% activity of wild type, could also be used.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

A suitable synthetic promoter is the HLA-DRA (termed hHLA) synthetic promoter having the following sequence:

(SEQ ID NO: 8)
TCGATTTCCGAATTCCACTATCCCTGTCTAGAAGT
CAGATTGGGGTTAAAGAGTCTGTCCGTGATTGACT
AACAGTCTTAAATACTTGATTTGTTGTTGTTGTTG
TCCTGTTTGTTTAAGAACTTTACTTCTTTATCCAA
TGAACGGAGTATCTTGTGTCCTGGACCCTTTGCAA
GAACCCTTCCCCTAGCAACAGATGCGTCATCTCAA
AATATTTTTCTGATTGGCCAAAGAGTAATTGATTT
GCATTTTAATGGTCAGACTCTATTACACCCCACAT
TCTCTTTTCTTTTATTCrTGTCTGTTCTGCCTCAC
TCCCGAGCTCTACTGACTCCCAACAGAGCGCCCAA
GAAGAAAACGC

As used herein, the terms “pseudotype”, “pseudotyped” or “pseudotyping”, or the like, refers to a virus whose viral envelope proteins have been substituted with those from a different virus. For example, HIV can be pseudotyped with vesicular stomatitis virus G-protein (VSV G) envelope proteins, which allows the HIV virus to infect a different or broader range of cells, as HIV envelope proteins normally only target CD4+ presenting cells.

As used herein, the term “tissue-specific promoter” refers to a promoter that selectively facilitates the expression of a gene of interest in a particular cell type or tissue type. Examples of tissue-specific promoters that may be used in conjunction with the compositions and methods of the disclosure include a sp146/p47 promoter, CD11b promoter, CD68 promoter, and a sp146/gp9 promoter, among others.

As used herein, the term “ubiquitous promoter” refers to a promoter that facilitates the expression of a gene of interest in a variety of cell types or tissue types, such as a promoter that does not exhibit a preference for facilitating gene expression in one cell type over another or in one tissue type over another. Examples of tissue-specific promoters that may be used in conjunction with the compositions and methods of the disclosure include human phosphoglycerate kinase (hPGK) promoter and an elongation factor 1-alpha promoter, among others.

As used herein, the term “inducible promoter” refers to a promoter that facilitates the expression of a gene of interest when activated by a specific stimulus, such as a substance or biomolecule. It is thus a regulated promoter, becoming active in response to specific stirnuli while a constitutive promoter is an unregulated promoter which is active in all circumstances. An inducible promoter may have sone basal transcriptional activity without stimulation but be hyper activated when stimulated.

A suitable inducible promoter is that from the human HLA-DRA gene, encoding the Class II Major Histocompatibility Complex (MHC-II) α-chain immunoglobulin domain of Histocompatibility Leukocyte Antigen, or a synthetic version derived from the endogenous promoter region. Class II molecules are constitutively expressed at a low basal level by professional antigen presenting cells, as microglia cells, and their expression is strongly upregulated during inflammation (Jenny Pan-Yun Ting et al. Cell. 109(2) suppl. 1, S21-33, Apr. 19, 2002).

The MHC class II promoters can be constitutively activated, as in B-cells, or can be induced by IFN-γ, as in macrophage and in many cells of parenchymal and mesenchymal origin, such as endothelial cells, oligodendrocytes, and skin fibroblasts. In both cases, the promoter activation involves the binding of the CIITA co-activator to a series of DNA-binding proteins that have cognate sites at the MHC class II promoters. The promoter for the human MHC class II heavy chain gene, HLA-DRA, is the most well studied, and in this case, the promoter contains sites, from 5′ to 3′, for the trimeric RFX, CREB, the trimeric NF-Y, Oct-1, and YY1. RFX, CREB, and NF-Y contribute to the binding of the CIITA co-activator, which is either constitutively expressed in cells that constitutively express the MHC class II genes, or is induced by IFN-g activation of STAT1a.

A suitable synthetic HLA-DRA derived promoter for use in the invention disclosed herein comprises or consists of the sequence disclosed in SEQ ID NO: 8, or one with at least 90%, such as at least 95% sequence identity thereto.

As used herein, the term “plasmid” refers to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “regulatory sequence” includes promoters, enhancers and other expression control elements (e.g., polyadenylation signals) that control the transcription or translation of the gene(s). Such regulatory sequences are described, for example, in Perdew et al., Regulation of Gene Expression (Humana Press, New York, N.Y., (2014)); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject. The term sample can also relate to a prepared or processed sample, such as a mRNA- or cDNA-containing sample.

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the term “transgene” refers to a recombinant nucleic acid (e.g., DNA or cDNA) encoding a gene product (e.g., a gene product described herein) that can be introduced into a host cell. The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “subject” and “patient” are used interchangeably and refer to an organism (e.g., a mammal, such as a human) that is at risk of developing or has been diagnosed as having, and/or is undergoing treatment for, a disease, such as FTD as described herein.

As used herein, the terms “transduction” and “transduce” refer to a method of introducing a gene or other polynucleotide sequence using a viral vector construct into a cell by means of viral infection rather than transfection. Successfully transduced cells should then be capable of expressing the introduced gene or other polynucleotide and/or the viral vector nucleic acid or part thereof in the cell.

As used herein a “transduction enhancer” (TE) is a compound or substance that is capable of facilitating an increase in the amount of viral transduction into a cell population. Typically, the transduction enhancer is added to the culture medium before or during transduction of the recipient cells (e.g. HSPCs). A continuously expanding list of small molecule compounds and peptides acting as transduction enhancers (TEs) have been identified. Mechanistically, these can be grouped into two major categories: (1) entry enhancers, which physically increase co-localization of or lower the repulsion between viral vector particles and target cells, or which trigger fusion (e.g. RetroNectin, LentiBOOST, protamine sulfate (PS), Vectofusin-1, ViraDuctin, and staurosporine [Stauro]), and (2) post entry TEs, ultimately yielding higher copy numbers of integrated vector by affecting intracellular processes, such as prostaglandin E2 (PGE2) and dimethyl prostaglandin E2 (dmPGE2).

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to or at risk of developing the condition or disorder, as well as those in which the condition or disorder is to be prevented.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Vectors that can be used for the expression of a protein or proteins described herein include plasmids that contain regulatory sequences, such as promoter and enhancer regions, which direct gene transcription. Additionally, useful vectors for expression of a protein or proteins described herein may contain polynucleotide sequences that enhance the rate of translation of the corresponding gene or genes or improve the stability or nuclear export of the mRNA that results from gene transcription. Examples of such sequence elements are 5′ and 3′ untranslated regions, an IRES, and a polyadenylation signal site in order to direct efficient transcription of a gene or genes carried on an expression vector. Expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin, among others.

### DETAILED DESCRIPTION OF THE INVENTION

The present invention provides compositions and methods for treating neurodegenerative diseases caused by dysfunctional expression of progranulin using HSPCs that express functional progranulin optionally in conjunction with overexpression of one or more metallothioneins.

In particular, the present invention provides compositions and methods for treating or preventing FTD and other neurodegenerative diseases that could benefit from overexpression of progranulin, such as ALS, Alzheimer's, and Parkinson's. The compositions and methods described herein may be used, for example, to treat a patient, such as an adult human patient, that is suffering from, for example FTD, as well as to prophylactically treat a patient at risk of developing FTD. Patients may be treated, for example, by providing to the patients one or more modified HSPCs, such as a population of cells, capable of expressing human progranulin. Without being limited by mechanism, the provision of such agents results in repopulation of microglia capable of producing progranulin, thus treating an underlying cause of the disease and reverse its pathophysiology. Thus, using the compositions and methods described herein, a patient may not only be treated in a manner that alleviates one or more symptoms associated with FTD, but also in a curative fashion or preventative fashion.

According to a first aspect of the invention there is provided a population of modified hematopoietic stem cells (HSPCs) where the cells carry an exogenous copy of a nucleic acid encoding functional progranulin. The extraneous copy can be one or more additional copies.

Suitably the cells have been modified so as to be capable of expressing functional progranulin. In particular embodiments, the expression level of progranulin is at levels higher than that of the unmodified cells.

Suitably the HSPC cells are mammalian cells. In a particular embodiment, the HSPCs are human cells. Prior to being modified the HSPCs or their precursors may have been isolated from a patient to be treated or from a different subject, such as a healthy volunteer, perhaps a matched donor.

In a particular embodiment, the HSPC is functionally equivalent to a microglia progenitor cell upon transplantation.

Suitably the nucleic acid encodes human progranulin.

In particular embodiments, the nucleic acid is a genomic DNA or a complementary DNA (cDNA).

In particular embodiments, the nucleic acid that encodes progranulin is the or a wild-type sequence.

In particular embodiments, the nucleic acid that encodes progranulin is codon-optimised.

In particular embodiments, the nucleic acid encoding human progranulin comprises the sequence of SEQ ID NO: 1, or a sequence with 95% sequence identity thereto.

In particular embodiments, the progranulin encoded by the exogenous copy of a nucleic acid comprises the sequence of SEQ ID NO: 2, or a sequence with 95% sequence identity thereto.

In a particular embodiment, the nucleic acid is integrated into the genome of the cells.

In a particular embodiment the nucleic acid is introduced into the cells via a viral vector.

The viral vector is suitably a retroviral vector, such as a lentiviral vector.

In particular embodiments, the viral vector is a lentiviral vector which comprises a progranulin encoding cDNA (GRN cDNA) under the control of a ubiquitous promoter. In a particular embodiment the promoter is the human phosphoglycerate kinase (hPGK) promoter. An exemplary human PGK nucleic acid sequence is disclosed in SEQ ID NO: 7.

In particular embodiments, the viral vector is a lentiviral vector which comprises a progranulin encoding cDNA (GRN cDNA) under the control of an inducible promoter. Suitably, the inducible promoter is activated by an inflammatory cytokine, such as interferon gamma.

By activated by, we mean that the promoter expresses more transgene when contacted by the inflammatory cytokine. In a particular embodiment the inducible promoter is the HLA-DRA promoter or a synthetic version derived therefrom which is capable of being induced by IFN-γ.

In particular embodiments the promoter is a cytokine-reactive promoter, such as one that is responsive to human interferon gamma (IFNγ), tumor necrosis factor alpha (TNFα) or interleukin 1 beta (IL1β). Fischer et al. (Xenotransplantaion. 27(6): e12634, 2020) describe certain cytokine-inducible promoters and their uses, such as CAG, ELAM, hCCL2 and pA20.

In particular embodiments, the promoter is responsive to (i.e. activated by) an inflammatory cytokine. Suitable promoters include HLA-DRA, CAG, ELAM, hCCL2 and pA20.

In particular embodiments, the promoter is HLA-DRA promoter or a synthetic version derived therefore. A suitable synthetic HLA-DRA derived promoter for use in the invention disclosed herein comprises or consists of the sequence disclosed in SEQ ID NO: 8, or one with at least 90%, such as at least 95% sequence identity thereto.

In particular embodiments the nucleic acid that encodes progranulin has been introduced by gene editing, such as CRISPR/cas, TALENs or Zinc finger methodology.

In addition to progranulin, the HSPCs may be modified to express metallothionein.

Thus, in particular embodiments, the HSPCs also comprise an exogenously introduced nucleic acid that encodes metallothionein, such as metallothionein 1G.

In particular embodiments that use viral vector transduction, the nucleic acid that encodes metallothionein is or can be part of the viral vector. In particular embodiments, the viral vector contains 1, 2, 3, 4, or 5 copies of the polynucleotide encoding metallothionein.

In particular embodiments the nucleic acid that encodes metallothionein has been introduced by gene editing, such as CRISPR/cas, TALENs or Zinc finger methodology.

The HSPCs to be modified can be obtained from various sources, such as from the bone marrow, G-CSF or plerixafor mobilized peripheral, umbilical cord, amniotic fluid, chorionic villi, cord blood, placental blood or blood. Such HSPC are herein referred to as source HSPCs. Of course, such source HSPC are at this stage unmodified. In a particular embodiment, the HSPCs to be modified are obtained from mobilized peripheral blood or bone marrow. The HSPCs to be modified can be derived from a healthy individual or an individual with a disease to be treated, such as someone with FTD.

Thus, in particular embodiments, the source HSPCs are obtained from a healthy individual or an individual with a diagnosed disease or disorder.

The source HSPCs are then used in standard viral transduction protocols or targeted gene addition protocols. In particular embodiments, the population of source HSPCs are ex vivo cultured before and/or during the introduction of the exogenous copy of a nucleic acid encoding progranulin, and optionally metallothionein. Typically, this is the time point when the viral vectors or viral particles housing the exogenous nucleic acid to be transferred to the cells, are cultured with the population of unmodified HSPCs. With transduction approaches the viral vector or viral particles may be added to cells in a multiplicity of infection (MOI) between about 1 and 200 such as about 100

According to the second aspect of the invention, the population of modified HSPC cells are produced by a method comprising:

a) contacting a sample of unmodified HSPCs with a nucleic acid carrying an exogenous copy of a nucleic acid encoding progranulin; and

b) allowing the nucleic acid encoding progranulin to integrate into the genome of the cells so as to produce a population of modified HSPCs. Suitably, the nucleic acid is part of a vector, such as a viral vector.

According to variant of the second aspect of the invention there is provided a method for producing a population of hematopoietic stem cells (HSPCs) modified to express progranulin, the method comprising, contacting a sample of HSPCs with a vector carrying an exogenous copy of a nucleic acid encoding progranulin under conditions to allow the vector to transduce the HSPCs.

Suitably the method is conducted ex vivo.

In an embodiment of this second aspect of the invention, the method further comprises establishing that there is at least one-fold increase in the expression level of progranulin in the modified cells compared to non-modified cells. Suitably, there is at least a two-, three-, four-, five, six-, seven-, eight, nine-, ten-fold or greater expression level of progranulin in the modified cells compared to non-modified cells. Suitably, at least 10%, 20%, 30%, 40%, 50%, 60%, 70%, 80%, 90% or more of the source HSPCs have been transduced by the viral vector.

In embodiments of this second aspect of the invention, before, during or after contact with the viral vector the HSPC are cultured in a medium comprising one or more transduction enhancers, such as one or more cytokines and/or PGE2 or dmPGE2.

In particular embodiments, the sample of HSPCs is obtained from bone marrow, umbilical cord, amniotic fluid, chorionic villi, cord blood, placental blood or peripheral blood.

In a particular embodiment, the sample of HSPCs is obtained from mobilized peripheral blood or bone marrow.

In particular embodiments, the sample of HSPCs is obtained from a healthy individual or from an individual with a neurodegenerative disease to be treated, such as FT). In a particular embodiment, the modified HSPCs or the HSPCs to be modified according to the methods of the invention are CD34⁺ and/or CD45⁺

In a particular embodiment, the vector is viral vector, such as a retroviral vector.

In a particular embodiment, the viral vector is a lentiviral vector.

In a particular embodiment, the nucleic acid is integrated into the genome of the cells.

In a particular embodiment, the nucleic acid carrying an exogenous copy of a nucleic acid encoding progranulin is introduced into the cell by gene editing, such as CRISPR/cas, TALENs or Zinc finger methodology.

According to the third aspect of the invention there is provided a population of HSPCs modified to express progranulin produced by any method of the invention.

According to a variation of the third aspect of the invention there is provided a composition comprising the modified HSPCs of the invention.

In one embodiment, the composition is suitable for transplantation into a subject.

According to a further aspect of the invention there is provided the HSPCs of the first aspect of the invention or the HSPC produced by the second aspect of the invention or a composition of the third aspect of the invention for use in therapy.

According to the fourth aspect of the invention there is provided a method of treating a neurodegenerative disease caused by a mutated progranulin, comprising administering to the subject the modified HSPCs of the invention or the composition according to the third aspect of the invention.

The neurodegenerative disease caused by a mutated progranulin can be frontotemporal dementia (FTD). The neurodegenerative disease caused by a mutated progranulin can be neuronal ceroid lipofuscinosis (NCL).

The invention further comprises a method for treating neurodegenerative diseases mediated by enhancing progranulin expression above endogenous levels. The invention thus embraces the treatment of neurodegenerative diseases such as frontotemporal dementia (FTD), neuronal ceroid lipofuscinosis (NCL), amyotrophic lateral sclerosis (ALS), Alzheimer's disease (AD) and Parkinson's disease (PD) by introducing into the patient (e.g., a mammalian patient, such as a human patient (e.g., an adult human patient)) in need thereof HSPCs modified to express progranulin.

According to a variation of the fourth aspect of the invention there is provided a method of treating frontotemporal dementia (FTD), comprising administering to the subject the modified HSPCs of the invention or the composition according to the third aspect of the invention.

According to a variation of the fourth aspect of the invention there is provided a method of treating neuronal ceroid lipofuscinosis (NCL), comprising administering to the subject the modified HSPCs of the invention or the composition according to the third aspect of the invention.

In a particular embodiment of this aspect of the invention, the introduction of the HSPCs results in an increase in granulin levels in brain tissue, such as in myeloid cells and microglia.

In one embodiment, the HSPCs are autologous to the recipient subject.

In other embodiments, the HSPCs are non-autologous and allogenic to the recipient subject.

In another embodiment, the HSPCs are non-autologous and xenogeneic to the recipient subject.

The modified HSPCs can be administered to a subject or patient in need thereof by any suitable means. In particular embodiments, the HSPCs are administered to the patient by IV, ICV or ITL injection, or any combination thereof.

According to a fifth aspect of the invention there is provided a population of progranulin expressing HSPCs of the first aspect of the invention or the HSPCs produced by the second aspect of the invention or the composition of the third aspect of the invention for use in the prevention or treatment of a neurodegenerative disorder mediated by a mutated progranulin or aberrant expression of progranulin.

According to a variation of the fifth aspect of the invention there is provided a population of progranulin expressing HSPCs of the first aspect of the invention or the HSPCs produced by the second aspect of the invention or the composition of the third aspect of the invention for use in the manufacture of a medicament for the prevention or treatment of a neurodegenerative disorder mediated by a mutated progranulin or aberrant expression of progranulin.

In particular embodiments the neurodegenerative disorder is frontotemporal dementia (FTD) or neuronal ceroid lipofuscinosis (NCL).

Nucleic Acids Encoding Functional Progranulin

In some embodiments, the lentiviral vector comprises a nucleic acid molecule that encodes a functional progranulin, such as human progranulin shown in SEQ ID NO: 2.

In some embodiments, the progranulin protein/polypeptide encoded by the nucleic acid molecule has at least 90% sequence identity (e.g., 90%, 91%, 92%, 93%, 94%, 95%, 96%, 97%, 98%, 99%, or 100% sequence identity) to the amino acid sequence of SEQ ID NO: 2.

MWTLVSWVALTAGLVAGTRCPDGQFCPVAC
CLDPGGASYSCCRPLLDKWPTTLSRHLGGP
CQVDAHCSAGHSCIFTVSGTSSCCPFPEAV
ACGDGHHCCPRGFHCSADGRSCFQRSGNNS
VGAIQCPDSQFECPDFSTCCVMVDGSWGCC
PMPQASCCEDRVHCCPHGAFCDLVHTRCIT
PTGTHPLAKKLPAQRTNRAVALSSSVMCPD
ARSRCPDGSTCCELPSGKYGCCPMPNATCC
SDHLHCCPQDTVCDLIQSKCLSKENATTDL
LTKLPAHTVGDVKCDMEVSCPDGYTCCRLQ
SGAWGCCPFTQAVCCEDHIHCCPAGFTCDT
QKGTCEQGPHQVPWMEKAPAHLSLPDPQAL
KRDVPCDNVSSCPSSDTCCQLTSGEWGCCP
IPEAVCCSDHQHCCPQGYTCVAEGQCQRGS
EIVAGLEKMPARRASLSHPRDIGCDQHTSC
PVGQTCCPSLGGSWACCQLPHAVCCEDRQH
CCPAGYTCNVKARSCEKEVVSAQPATFLAR
SPHVGVKDVECGEGHFCHDNQTCCRDNRQG
WACCPYRQGVCCADRRHCCPAGFRCAARGT
KCLRREAPRWDAPLRDPALRQLL

[=isoform 1 which is the canonical sequence, but there are other isoforms]

In some embodiments, the progranulin polypeptide/protein has an amino acid sequence that differs from the amino acid sequence of SEQ ID NO: 2 by way of one or more conservative amino acid substitutions (e.g., by way of from 1 to 50 conservative amino acid substitutions).

In some embodiments, the progranulin polypeptide has the amino acid sequence of SEQ ID NO: 2.

In some embodiments, the nucleic acid molecule that encodes the progranulin has at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 1.

In some embodiments, the nucleic acid molecule that encodes the progranulin has the nucleotide sequence of SEQ ID NO: 1.

In some embodiments, the nucleic acid molecule that encodes the progranulin is a codon-optimised version of the nucleotide sequence of SEQ ID NO: 1. An exemplary codon optimised version is disclosed in SEQ ID NO: 6.

Nucleic Acids Encoding Metallothionein

Metallothioneins

Metallothioneins (MTs) belong to the group of intracellular cysteine-rich, metal-binding proteins and have been involved in homeostasis of essential metals such as zinc and copper, detoxification of toxic metals such as cadmium, and protection against oxidative stress. Furthermore, the MTs have been implicated in processes of neuroprotection and neuroregeneration in several conditions, including AD and LSD (Juarez-Rebollar et al., Oxid Med Cell Longev. 2017: 5828056, doi: 10.1155/2017/5828056; Ruttkay-Nedecky et al., Int. J Mol Sci. 14(3):6044-66, 2013).

Some embodiments of the present disclosure provide a modified HSPC cell that expresses a functional progranulin (as described herein) and optionally a metallothionein encoded by an exogenous nucleic acid molecule as well. The metallothionein is a MTI, MT2, MT3, or MT4 metallothionein in some embodiments. In some embodiments, the metallothionein is MTI. In some embodiments the MTI metallothionein is a MTIA, MTIB, MTIE, MTIF, MTIG, MTIH, MTIL, MTIM, or a MTIX metallothionein. In some embodiments, a cell expresses more than one metallothionein from an exogenous nucleic acid molecule. In some embodiments, a cell expresses MTIG from an exogenous nucleic acid molecule. In some embodiments, the cell is an HSPC or its progeny. In some embodiments, the cell is a microglia or microglia-like cell. Metallothioneins are described in International Application Nos. WO2018/136434 and WO2018/136435, the contents of which are incorporated herein by reference in their entirety.

Some embodiments of the present disclosure provide a modified cell that expresses progranulin and a metallothionein encoded by exogenous nucleic acid molecules. In some embodiments, the metallothionein is a MTI, MT2, MT3, or MT4 metallothionein. In some embodiments, the modified cell expresses a MTIA, MTIB, MTIE, MTIF, MTIG, MTIH, MTIL, MTIM, or a MTIX metallothionein and progranulin. In some embodiments, the modified cell expresses MTIG and progranulin from exogenous nucleic acid molecules. In some embodiments, the cell is an HSPC or its progeny. In some embodiments, the cell is a microglia or microglia-like cell.

In some embodiments, the HSPC has been modified to comprise a nucleic acid molecule that encodes one or more copies of metallothionein, such as metallothionein 1G shown in SEQ ID NO: 3. Suitably the HSPC is modified to contain 1, 2, 3, 4, 5, 6, or more copies of a nucleic acid molecule that encodes a metallothionein.

In some embodiments, the progranulin protein/polypeptide encoded by the nucleic acid molecule has at least 90% sequence identity (e.g., 90%, 91%, 92%, 93%, 94%, 95%, 96%, 97%, 98%, 99%, or 100% sequence identity) to the amino acid sequence of SEQ ID NO: 3.

In some embodiments, the metallothionein 1G has the amino acid sequence of SEQ ID NO: 3.

In some embodiments, the nucleic acid molecule that encodes the metallothionein 1G has at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 4.

In some embodiments, the nucleic acid molecule that encodes the metallothionein has the nucleotide sequence of SEQ ID NO: 4.

Source Cells

Microglia have a developmental origin distinct from that of bone marrow-derived myelomonocytes (Ginhoux et al. Science 330, 841-845 (2010), the contents of which are incorporated herein by reference in their entirety). However, cells having a microglia-like phenotype can be derived from transplanted donor HSPCs. HSPCs capable of generating microglia-like cells upon transplantation into myeloablated recipients are retained within human and murine long-term hematopoietic stem and progenitor cells (HSPCs), thereby providing a reservoir of pluripotent cells capable of differentiating into therapeutic microglia for the treatment of the neurodegenerative diseases mentioned herein.

Suitable cells that may be used in conjunction with the compositions and methods described herein include hematopoietic stem cells (HSCs) and hematopoietic progenitor cells (HPCs), that are capable of undergoing further differentiation. As used herein, HSCs and HPCs either as individual populations of cells or mixed together are herein also referred to as HSPCs. HSCs are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34⁺. In addition, HSCs also refer to long term repopulating HSC (LT-HSC) and short-term repopulating HSC (ST-HSC). Any of these HSCs can be used in conjunction with the compositions and methods described herein.

HSCs and other pluripotent progenitors can be obtained from blood products. A blood product is a product obtained from the body or an organ of the body containing cells of hematopoietic origin. Such sources include unfractionated bone marrow, umbilical cord, placenta, peripheral blood, or mobilized peripheral blood. All of the aforementioned crude or unfractionated blood products can be enriched for cells having HSC or myeloid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and re-establish productive and sustained hematopoiesis. Such selection is accomplished using, for example, commercially available magnetic anti-CD34 beads (Dynal, Lake Success, N.Y.). Myeloid progenitor cells can also be isolated based on the markers they express. Unfractionated blood products can be obtained directly from a donor or retrieved from cryopreservative storage. HSCs and myeloid progenitor cells can also be obtained from by differentiation of ES cells, iPS cells or other reprogrammed mature cells types.

Cells that may be used in conjunction with the compositions and methods described herein include CD34⁺/CD90⁺ cells, CD34⁺ CD38⁻ cells and CD34⁺/CD164⁺ cells. These cells may contain a higher percentage of HSCs. These cells are described in WO2015/059674, WO2017/218948, Radtke et al. Sci. Transl. Med. 9: 1-10, 2017, and Pellin et al. Nat. Comm. 1-: 2395, 2019, the disclosures of each of which are hereby incorporated by reference in their entirety.

Other suitable cells that may be used in conjunction with the compositions and methods described herein include those that are CD45⁺.

In some embodiments, the patient undergoing treatment is the donor that provides cells (e.g., pluripotent cells, such as CD34⁺ hematopoietic stem and/or progenitor cells) that are subsequently modified to express progranulin according to the invention before being re-administered to the patient. In such cases, withdrawn cells (e.g., HSPCs) may be re-infused into the subject following, for example, incorporation of a transgene encoding functional progranulin, such that the cells may subsequently home to hematopoietic (and CNS) tissue and establish productive hematopoiesis, thereby populating or repopulating a line of cells (including the generation of microglia equivalent cells) that are defective or deficient in the patient. In cases in which the patient undergoing treatment also serves as the cell donor, the transplanted cells (e.g., HSPCs) are less likely to undergo graft rejection. This stems from the fact that the infused cells are derived from the patient and express the same HLA class I and class II antigens as expressed by the patient. Alternatively, the patient and the donor may be distinct. In some embodiments, the patient and the donor are related, and may, for example, be HLA-matched (matched donor). As described herein, HLA-matched donor-recipient pairs have a decreased risk of graft rejection, as endogenous T cells and NK cells within the transplant recipient are less likely to recognize the incoming hematopoietic stem or progenitor cell graft as foreign and are thus less likely to mount an immune response against the transplant. Exemplary HLA-matched donor-recipient pairs are donors and recipients that are genetically related, such as familial donor-recipient pairs (e.g., sibling donor-recipient pairs). In some embodiments, the patient and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

Suitably, the HSPC that may be used in conjunction with the compositions and methods described herein act as being functionally equivalent to a microglia progenitor cell upon transplantation.

Thus, HSPCs that may be used in conjunction with the compositions and methods described herein include allogeneic cells and autologous cells. When allogeneic cells are used, the cells may optionally be HLA-matched to the subject receiving a cell treatment.

Modifying Cells

To produce the modified HSPC cells described herein, any approach known in the art may be used to genetically engineer the cell to be able to express a functional progranulin. For example, cells may be modified by gene editing to replace a functional gene for an endogenous damaged gene, such as GRN. Alternatively, a functional gene can be introduced elsewhere in the cell genome so as to produce functional progranulin. “Gene editing” tools can manipulate a cell's DNA sequence at a specific chromosomal locus without introducing mutations at other sites of the genome. This technology effectively enables a researcher to manipulate the genome of a cell in vitro or in vivo.

In one embodiment, gene editing involves targeting an endonuclease to a specific site in a genome to generate a double strand break at the specific location. If a donor DNA molecule (e.g., a plasmid or oligonucleotide) is introduced, interactions between the nucleic acid comprising the double strand break and the introduced DNA can occur, especially if the two nucleic acids share homologous sequences. In this instance, a process termed “gene targeting” can occur, in which the DNA ends of the chromosome invade homologous sequences of the donor DNA by homologous recombination. By using the donor plasmid sequence as a template for homologous recombination, a seamless knock out of the gene of interest can be accomplished. Importantly, if the donor DNA molecule includes a deletion within the target gene (e.g., GRN), homologous recombination-mediated double strand break repair will introduce the donor sequence intothe chromosome, resulting in the deletion being introduced within the chromosomal locus. By targeting the nuclease to a genomic site that contains the target gene, the concept is to use double strand break formation to stimulate homologous recombination and to thereby replace the dysfunctional target gene with a functional form of the gene. The advantage of the homologous recombination pathway isthat it has the potential to seamlessly swap out an endogenous dysfunctional gene with a functional, such as wild-type version at the locus of the endogenous gene. Alternatively, the dysfunctional gene can be left in place and the functional gene introduced at a different chromosomal location.

Genome editing tools may use double strand breaks to enhance gene manipulation of cells. Such methods can employ zinc finger nucleases, described for example in U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; and 6,479,626; and U.S. Pat. Publ. Nos. 20030232410 and US2009020314, which are incorporated herein by reference); Transcription Activator-Like Effector Nucleases (TALENs; described for example in U.S. Pat. Nos. 8,440,431; 8,440,432; 8,450,471; 8,586,363; and 8,697,853; and U.S. Pat. Publ. Nos. 20110145940; 20120178131; 20120178169; 20120214228; 20130122581; 20140335592; and 20140335618; which are incorporated herein by reference), and the CRISPR (Clustered Regularly Interspaced Short Palindromic Repeats)/Cas9 system (described for example in U.S. Pat. Nos. 8,697,359; 8,771,945; 8,795,965; 8,871,445; 8,889,356; 8,906,616; 8,932,814; 8,945,839; 8,993,233; and 8,999,641; and U.S. Pat. Publ. Nos. 20140170753; 20140227787; 20140179006; 20140189896; 20140273231; 20140242664; 20140273232; 20150184139; 20150203872; 20150031134; 20150079681; 20150232882; and 20150247150,

which are incorporated herein by reference). For example, zinc finger nuclease DNA sequence recognition capabilities and specificity can be unpredictable. Similarly, TALENs and CRISPR/Cas cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. Zinc finger nucleases and TALENs entail use of modular sequence-specific DNA binding proteins to generate specificity for about 18 bases sequences in the genome RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break. The zinc finger nuclease is then repaired either by non-homologous end joining, which leads to insertion/deletion (indel) mutations, or by homology-directed repair, which requires an exogenous template and can generate a precise modification at a target locus (Mali et al., Science, Feb. 15, 2013; 339 (6121): 823-6, the contents of which are herein by reference in their entirety). Unlike gene therapy methods that add a functional, or partially functional, copy of a gene to a subject's cells but retain the original dysfunctional copy of the gene, this system can remove the defect in the dysfunctional copy. Genetic correction using modified nucleases has been demonstrated in tissue culture cells and rodent models of rare diseases.

CRISPR has been used in a wide range of organisms including baker's yeast (S. cerevisiae), zebra fish, nematodes (e.g., C. elegans), plants, mice, and several other organisms. Additionally, CRISPR has been modified to make programmable transcription factors that allow scientists to target and activate or silence specific genes. Libraries of tens of thousands of guide RNAs are now available. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length, with some CRISPR spacer sequences exactly matching sequences from plasmids and phages, although some spacers match the prokaryote's genome (self-targeting spacers). New spacers can be added rapidly in response to phage infection. CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define eight CRISPR subtypes (E coli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about thirty base pairs in length), which are then inserted into the CRISPR locus near the leader sequence. RNAs from the CRISPR loci are constitutively expressed and are processed by Cas proteins to small RNAs comprising individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes CRISPR transcripts. Interestingly, CRISPR-based phage inactivation in E. coli requires Cascade and Cas3, but neither CasI nor Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V-restriction enzymes. See also U.S. Patent Publication 2014/0068797, which is incorporated by reference in its entirety As an RNA guided protein, Cas9 requires an RNA molecule to direct the recognition of DNA targets. Though Cas9 preferentially interrogates DNA sequences containing a protospacer adjacent motif (PAM) sequence (i.e., NGG). However, the Cas9-gRNA complex requires a substantial complementarity between the guide RNA (gRNA) and the target nucleic acid sequence to create a double strand break. Synthetic gRNA can be designed to combine the essential RNA sequences for Cas9 targeting into a single RNA expressed with the RNA polymerase type 21 promoter U6 driving expression. Synthetic gRNAs are slightly over 100 bases at the minimum length and contain a portion which is targets the 20 protospacer nucleotides immediately preceding the PAM sequence NGG. In one approach, an HSPC cell is altered to introduce a functional progranulin gene. In another approach, an HSPC cell is altered to replace a dysfunctional progranulin gene with a functional version using a CRISPR-Cas system. Cas9, for example, can be used to target a progranulin gene. Upon target recognition, Cas9 induces double strand breaks in the progranulin target gene. Homology-directed repair (HDR) at the double-strand break site can allow insertion of a functional form of the progranulin sequence. The following US patents and patent publications are incorporated herein by reference: U.S. Pat. No. 8,697,35; 20140170753; 20140179006; 20140179770; 20140186843; 20140186958; 20140189896; 20140227787; 20140242664; 20140248702; 20140256046; 20140273230; 20140273233; 20140273234; 20140295556; 20140295557; 20140310830; 20140356956; 20140356959; 20140357530; 20150020223; 20150031132; 20150031133; 20150031134; 20150044191; 20150044192; 20150045546; 20150050699; 20150056705; 20150071898; 20150071899; 20150071903; 20150079681; 20150159172; 20150165054; 20150166980; and 20150184139.

In particular embodiments, the HSPCs are edited to express an exogenous progranulin using CRISPR, TALENs or Zinc finger nuclease methodology.

Use of Hemizygous CX3CRI Cells

The modified HSPCs of the invention can act as vehicles to deliver progranulin to the brain. The speed of implantation and therapeutic efficacy can be affected by the pace of reconstitution of resident microglia by the transplanted modified HSPCs.

Several reports propose the CX3CL1/CX3CR1 axis as a potential target for therapeutic intervention in the context of neurodegenerative diseases.

CX3CR1, also known as the fractalkine receptor, is a seven-transmembrane domain receptor belonging to G protein-coupled receptors family which is expressed on microglia that binds Fractalkine (CX3CL1) and regulates microglia recruitment to sites of neuroinflammation, Being a G protein-coupled receptor, CX3CR1's role is mostly inhibitory as it acts to reduce production of cAMP and prevent triggering signalling cascades mediated by second messengers. Microglia are the only cell in the central nervous system that express CX3CR1, which they express at high levels, particularly during development and in response to brain damage/pathology.

Activation of the CX3CR1-CX3CL1 axis leads to maintenance of microglia in a quiescent state and of homeostasis in the neuronal network. Under physiological conditions, CX3CL1 seems to inhibit microglia activation, while in particular conditions a paradoxical promotion of an inflammatory response may occur. Neurons are the greater producers of CX3CL1 in the brain and this axis is important for communication with microglia cells. Astrocytes (GFAP⁺) also display constitutive mRNAexpression forCX3CL1. Endothelial cells in the brain and spinal cord, as opposed to those in other locations, do not present constitutive CX3CL1 expression on the surface, which suggests that it is rather dependent on their activation. CX3CL1 and CX3CR1 are also expressed in the choroid plexus.

The inventor has found that (i) transplantation of total bone marrow or HSPCs from donor mice haplo-insufficient for the CX3CR1 gene results in an greater and faster appearance of microglia like donor cells in the recipients' brain, as compared to standard wild type donors, and that (ii) in the context of competitive transplantation, haplo-insufficient donor derived cells contribute to a greater extent as compared to wildtype donor cells to the repopulation of the hematopoietic organs and brain myeloid compartment of the recipients.

Thus, transplantation of CX3CR1 haploinsufficient HSPCs capable of expressing functional progranulin is expected to result in a more robust and faster myeloid engraftment and generation of bona fide microglia.

Thus, the present disclosure contemplates isolating HSPCs and knocking out one allele of CX3CR1 to create a hemizygous cell, which can be cultured to generate a population of cells that are hemizygous for CX3CR1. Such CX3CR1 hemizygous HSPCs can then be modified to incorporate a nucleic acid sequence encoding progranulin and optionally metallothionein protein in the missing CX3CR1 allele locus. These cDNAs will be expressed by the CX3CR1 locus. In this way, the hemizygous HSPCs are manipulated to express a therapeutic agent in a regulated and microglia specific manner. Alternatively, an isolated HSPC may be edited to remove one copy of CX3CR1 to generate a hemizygous HSPC. Editing a single copy of the CX3CR1 comprises, in some embodiments, replacing the CX3CR1 allele with an exogenous nucleic acid molecule encoding a therapeutic agent. Such editing is carried out using any method known in the art.

In various embodiments of the above aspects, the source HSPCs are first modified to be hemizygous for the CX3CR1 gene.

In one approach, an HSPC cell is altered to delete or inactivate a CX3CR1 allele using a CRISPR-Cas system. Cas9 can be used to target a CX3CR1 gene. Upon target recognition, Cas9 induces double strand breaks in the CX3CR1 target gene. Homology-directed repair (HDR) at the double-strand break site can allow insertion of an inactive or deleted form of the CX3CR1 sequence.

Expression of Exogenous Therapeutic Agents

Progranulin and metallothioneins are therapeutic polypeptides useful for the treatment of neurodegenerative diseases. Polynucleotide encoding such proteins can be inserted into expression vectors by techniques known in the art. For example, double stranded DNA can be cloned into a suitable vector by restriction enzyme linking involving the use of synthetic DNA linkers or by blunt-ended ligation. DNA ligases are usually used to ligate the DNA molecules and undesirable joining can be avoided by treatment with alkaline phosphatase.

The present invention also contemplates recombinant vectors (e.g., recombinant plasmids or recombinant expression vectors) that include nucleic acid molecules encoding progranulin alone or along with one or more copies of metallothionein as described herein. The term “recombinant vector” includes a vector (e.g., plasmid, phage, phasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. For example, a recombinant vector may include a nucleotide sequence encoding a polypeptide, or fragment thereof, operatively linked to regulatory sequences such as promoter sequences, terminator sequences, long terminal repeats, untranslated regions, and the like, as defined herein. Recombinant expression vectors allow for expression of the genes or nucleic acids included in them.

In some embodiments of the present disclosure, one or more DNA molecule having a nucleotide sequence encoding one or more polypeptides or polynucleotides described herein are operatively linked to one or more regulatory sequences, which can integrate the desired DNA molecule into a eukaryotic cell. Cells (e.g., HSPCs, microglia) which have been stably transfected or transduced by the introduced DNA can be selected, for example, by introducing one or more markers which allow for selection of host cells which contain the expression vector. A selectable marker gene can either be linked directly to a nucleic acid sequence to be expressed or be introduced into the same cell by co-transfection or co-transduction. Any additional elements needed for optimal synthesis of polynucleotides or polypeptides described herein would be apparent to one of ordinary skill in the art.

In some embodiments, an HSPC may be modified by introducing an exogenous nucleic acid molecule into the cell. The exogenous nucleic acid may comprise a transgene encoding a therapeutic agent for the treatment of the neurodegenerative disease, such as FTD. The exogenous nucleic acid, in some embodiments, comprises regulatory elements for expressing a transgene. For example, an exogenous nucleic acid molecule may comprise a transgene encoding a therapeutic agent for the treatment of the neurodegenerative disease, such as FTD, and a promoter for expressing the transgene. In some embodiments, the promoter is a constitutively active promoter such as, for example, the cytomegalovirus (CMV), simian virus 40 (SV40) promoter or PGK.

In some embodiments, the promoter may be a tissue-specific promoter, wherein the transgene is expressed upon engraftment and differentiation of the HSPC. For example, tetracycline is a drug that can be used to activate a tetracyclin-sensitive promoter. In some embodiments, a neuronal specific promoter is the synapsin (Syn) promoter.

In some embodiments, the promoter may be an inducible promoter, wherein the transgene is expressed only in the presence or absence of a particular compound. Alternatively, the promoter may be an inducible promoter, wherein expression of the transgene is enhanced in the presence of a particular compound.

In a particular embodiment the promoter is HLA-DRA promoter or one derived therefrom, which is regulated by inflammatory cytokines such as interferon gamma.

Using an HLA-DRA promoter the inventor has demonstrated that expression of the progranulin transgene is enhanced when in the presence of the inflammatory cytokine interferon gamma. Interferon gamma and other cytokines levels are increased in inflammatory conditions. The use of a promoter that regulates progranulin expression based on local inflammatory signals has the potential to deliver more effective treatment because the amount of progranulin/granulin produced by the modified cells will respond to the local neuroinflammatory situation.

In some embodiments, microglia or microglia-like cells derived from an HSPC comprising a transgene driven by a brain-specific promoter transplanted into the brain of a subject will express the transgene.

In some embodiments, the exogenous nucleic acid molecule may comprise, in addition to a transgene, a detectable label or other marker that allows identification of cells that have been successfully modified or that are derived from cells that have been successfully modified to express the transgene.

Methods of introducing exogenous nucleic acid molecules into a cell are known in the art. For example, eukaryotic cells can take up nucleic acid molecules from the environment via transfection (e.g., calcium phosphate-mediated transfection). Transfection does not employ a virus or viral vector for introducing the exogenous nucleic acid into the recipient cell. Stable transfection of a eukaryotic cell comprises integration into the recipient cell's genome of the transfected nucleic acid, which can then be inherited by the recipient cell's progeny.

Eukaryotic cells (i.e., HSPCs) can be modified via transduction, in which a virus or viral vector stably introduces an exogenous nucleic acid molecule to the recipient cell. Eukaryotic transduction delivery systems are known in the art. Transduction of most cell types can be accomplished with retroviral, lentiviral, adenoviral, adeno-associated, and avian virus systems, and such systems are well-known in the art. While retroviruses systems are generally not compatible with neuronal cell transduction, lentiviruses are a genus of retroviruses well-suited for transducing stem cells as well as neuronal cells. Thus, in some embodiments of the present disclosure, the viral vector system is a lentiviral system. In some embodiments, the viral vector system is an avian virus system, for example, the avian viral vector system described in WO2010/130844, the contents of which is incorporated herein by reference in their entirety. In some embodiments, the viral vectors are assembled or packaged in a packaging cell prior to contacting the intended recipient cell. In some embodiments, the vector system is a self-inactivating system, wherein the viral vector is assembled in a packaging cell, but after contacting the recipient cell, the viral vector is not able to be produced in the recipient cell.

The components of a viral vector are typically encoded on plasmids, and because efficiencies of transduction decrease with large plasmid size, multiple plasmids that have different viral sequences necessary for packaging may be necessary. For example, in a lentiviral vector system, a first plasmid may comprise a nucleotide sequence encoding a Group antigens (gag) and/or a reverse transcriptase (pol) gene, while a second plasmid encodes regulator of expression of virion proteins (rev) and/or envelope (env) genes. The exogenous nucleic acid molecule comprising a transgene can be packaged into the vector and delivered into a recipient cells where the transgene is integrated into the recipient cell's genome. Additionally, the transgene may be packaged using a split-packaging system as described in WO2010/130844.

After the introduction of one or more vector(s), host cells are cultured prior to administration to a subject. In some embodiments, the expression of recombinant proteins can be detected by immunoassays including Western blot analysis, immunoblot, and immunofluorescence. Purification of recombinant proteins can be carried out by any of the methods known in the art or described herein, for example, any conventional procedures involving extraction, precipitation, chromatography, and electrophoresis. A further purification procedure that may be used for purifying proteins is affinity chromatography using monoclonal antibodies which bind a target protein. Generally, crude preparations containing a recombinant protein are passed through a column on which a suitable monoclonal antibody is immobilized. The protein usually binds to the column via the specific antibody while the impurities pass through. After washing the column, the protein is eluted from the gel by changing pH or ionic strength, for example.

Generation of Modified HSPCs by Viral Transduction

Priming of Cells

In some embodiments, the source HSPCs are expanded ex vivo by contacting with one or more cell expansion agents described herein or known in the art to promote cell proliferation, thereby yielding a population of cells for transduction. For example, the expansion agent may be StemRegenin 1, also known in the art as compound SR1, that is disclosed in U.S. Pat. Nos. 8,927,281 and 9,580,426, the disclosures of each of which are incorporated herein by reference in their entirety.

Further expansion agents that may be used in conjunction with the compositions and methods of the disclosure include compound UM-171, which is described in U.S. Pat. No. 9,409,906, the disclosure of which is incorporated herein by reference in its entirety and histone deacetylase (HDAC) inhibitors, as described, for example, in WO 2000/023567, the disclosure of which is incorporated herein by reference

In some embodiments, prior to isolation of the source cells from the patient or donor, the patient or donor is administered one or more pluripotent cell mobilization agents that stimulate the migration of pluripotent cells (e.g., CD34⁺ HSCs and HPCs) from a stem cell niche, such as the bone marrow, to peripheral circulation. Exemplary cell mobilization agents that may be used in conjunction with the compositions and methods of the disclosure are described herein and known in the art. For example, the mobilization agent may be a C—X—C motif chemokine receptor (CXCR) 2 (CXCR2) agonist, such as Gro-beta, or a truncated variant thereof (see for example, U.S. Pat. Nos. 6,080,398; 6,447,766; and 6,399,053, the disclosures of each of which are incorporated herein by reference in their entirety). or Alternatively, the mobilization agent may be a CXCR4 antagonist, such as plerixafor or a variant thereof. Plerixafor and structurally similar compounds are described, for example, in U.S. Pat. Nos. 6,987,102; 7,935,692; and 7,897,590, the disclosures of each of which are incorporated herein by reference. Additionally, or alternatively, the mobilization agent may be a granulocyte colony-stimulating factor (G-CSF). The use of G-CSF as an agent to induce mobilization of pluripotent cells (e.g., CD34⁺ HSCs and/or HPCs) from a stem cell niche to peripheral circulation is described, for example, in US 2010/0178271, the disclosure of which is incorporated herein by reference in its entirety.

Viral Vector

Suitably, the source HSPCs are transduced ex vivo with a viral vector to form cells capable of expressing functional progranulin.

Eukaryotic cells (i.e., HSPCs) can be modified via transduction, in which a virus or viral vector stably introduces an exogenous nucleic acid molecule to the recipient cell. Eukaryotic transduction delivery systems are known in the art. Transduction of most cell types can be accomplished with retroviral, lentiviral, adenoviral, adeno-associated, and avian virus systems, and such systems are well-known in the art.

In some embodiments, the viral vector system is an avian virus system, for example, the avian viral vector system described in WO2010/130844, the contents of which is incorporated herein by reference in their entirety.

Suitably the vector used in the methods and compositions described herein is a retroviral vector. One type of retroviral vector that may be used in the methods and compositions described herein is a lentiviral vector. Lentiviral vectors (LVs), a subset of retroviruses, transduce a wide range of dividing and non-dividing cell types, including neuronal cells, with high efficiency, conferring stable, long-term expression of the transgene. An overview of optimization strategies for packaging and transducing LVs is provided in Delenda, The Journal of Gene Medicine 6: S125 (2004), the disclosure of which is incorporated herein by reference.

Thus, in some embodiments, the viral vector is a Retroviridae family viral vector, such as a lentiviral vector. In some embodiments, the viral vector is a lentivirus/lentiviral vector system.

In some embodiments, the lentiviral vector is derived from a lentivirus selected from the group consisting of: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In particular embodiments, the lentiviral vector is derived from an HIV lentivirus, such as HIV-1 lentivirus.

In some embodiments, the viral vectors are assembled or packaged in a packaging cell prior to contacting the intended recipient cell. In some embodiments, the vector system is a self-inactivating system, wherein the viral vector is assembled in a packaging cell, but after contacting the recipient cell, the viral vector is not able to be produced in the recipient cell.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans co-expression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The lentiviral vector may further include a pHR′ backbone, which may include for example as provided below. It will be readily apparent to one skilled in the art that optionally one or more of these regions is substituted with another region performing a similar function.

The Lentigen LV described in Lu et al., Journal of Gene Medicine 6:963 (2004) may be used to express the DNA molecules and/or transduce cells.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

In some embodiments, the Retroviridae family viral vector contains a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR. In some embodiments, the 5′ LTR is modified to comprise a deletion compared to the wild-type 5′ LTR.

In particular embodiments, the promoter of the 5′ LTR is replaced with a heterologous promoter selected from the group consisting of: a phosphoglycerate kinase (PGK) promoter, a TSPO promoter, a HLA-DRA promoter, a cytomegalovirus (CMV) promoter, a Rous Sarcoma Virus (RSV) promoter, or a Simian Virus 40 (SV40) promoter.

In a particular embodiment, the heterologous gene (e.g. coding for progranulin) is under the control of a phosphoglycerate kinase (PGK) promoter, such as human PGK promoter.

In a particular embodiment, the heterologous gene (e.g. coding for progranulin) is under the control of a HLA-DRA promoter. A suitable synthetic HLA-DRA derived promoter for use in the invention disclosed herein comprises or consists of the sequence disclosed in SEQ ID NO: 8, or one with at least 90%, such as at least 95% sequence identity thereto.

In a particular embodiment, the heterologous gene (e.g. coding for progranulin) is under the control of a hTSPO promoter. A suitable synthetic TSPO promoter for use in the invention disclosed herein comprises or consists of the sequence disclosed in SEQ ID NO: 9, or one with at least 90%, such as at least 95% sequence identity thereto.

In further embodiments, the RNA export element comprises a hepatitis B virus post-transcriptional regulatory element (PRE) or a human immunodeficiency virus (HIV) rev response element (RRE).

In certain embodiments, the 3′ LTR comprises a polyadenylation sequence. In particular embodiments, the lentiviral vector encodes an ATP-binding cassette, sub-family D, member 1 (ABCD1) polypeptide.

In further embodiments, the lentiviral vector comprises a myeloproliferative sarcoma virus enhancer, negative control region deleted, dl587rev primer-binding site substituted (MND) promoter or transcriptionally active fragment thereof operably linked to a polynucleotide encoding an ATP-binding cassette, sub-family D, member 1 (ABCD1) polypeptide.

Viral envelope proteins (env) determine the range of host cells which can ultimately be infected/transduced by recombinant retroviruses (including lentiviruses) generated from cell lines.

The viral vectors used in the present invention may result from “pseudotype” formation, where co-infection of a cell by different viruses produces progeny virions containing the genome of one virus encapsulated by the envelope proteins of another. The use of pseudotyping broadens the host range of the virus by utilising the viral entry mechanism of the encapsulating virus.

Pseudotyping of lentiviral particles, such as those based on HIV, with vesicular stomatitis virus (VSV) proteins (including VSV G) have proved popular.

In some embodiments, the viral vector is a pseudotyped viral vector, such as a pseudotyped viral vector. In some embodiments, the viral vector is an HIV-1 vector that is VSV G pseudotyped.

The vector construct can be transfected into a suitable packaging cell line to produce infectious viral particles that can be used to transduce the source HSPCs. The production of infectious viral particles and viral stock solutions can be carried out using conventional techniques. Methods for preparing viral stock solutions are known in the art, e.g. Soneoka et al. Nucl. Acids Res. 23:628-633, 1995; Landau et al. J Virol. 66:5110-5113, 1992.

In various embodiments, the present invention contemplates, in part, a population of HSPCs transduced with a viral vector (e.g. lentivirus/lentiviral vector), wherein at least 50% of the source cells are transduced. Suitably there is an average vector copy number (VCN) of about 0.5 to about 5.0. Suitably the viability of the population of hematopoietic cells is at least 50%, such as at least 60%, at least 75%.

In particular embodiments, the lentiviral vector transduces the HSPCs at a multiplicity of infection (MOI) of about 1 to about 250.

In particular embodiments, the lentiviral vector transduces the HSPCs at a multiplicity of infection (MOI) of about 10 to about 100.

In some embodiments, the MOI is about 1, about 3, about 5, about 10, about 20, about 30, about 40, about 50, about 70, about 100, about 125, about 150, about 175, about 200, or more.

As can be seen in the Examples, varying the MOI can influence the vector copy number in the transduced cells. Accordingly, in particular embodiments the lentiviral vector transduces the HSPCs at a MOI that correlates with a desired vector copy number to be achieved.

In certain embodiments, at least 25% of the cells have been transduced.

In certain embodiments, at least 50% of the cells have been transduced.

In certain embodiments, at least 75% of the cells have been transduced.

In additional embodiments, at least 90% of the cells have been transduced.

In particular embodiments, the average VCN is at least 1.0.

In particular embodiments, the average VCN is at least 1.5.

In particular embodiments, the average VCN is at least 2.0.

In particular embodiments, the average VCN is at least 2.5.

In particular embodiments, the average VCN is at least 5.

In particular embodiments, the average VCN is at least 10.

In particular embodiments, the average VCN is at least 1.5.

In particular embodiments, when using a constitutive promoter, such as PGK, the average VCN is between 1 and 5, such as 1, 2, 3, 4 or 5.

In particular embodiments, when using an inducible promoter, such as hHLA comprising SEQ ID NO:8, or a sequence with at least 90% sequence identity thereto, the average VCN is between 3 and 10, such as 3, 4, 5, 6, 7, 8, 9 and 10. A range between 4 and 8 is suitable.

In particular embodiments, the invention utilises an hTSPO promoter, such as hTSPO comprising SEQ ID NO: 9, or a sequence with at least 90% sequence identity thereto. Suitably, when using this type of promoter, the desired average VCN in the transduced HSPCs is between 3 and 10, such as 3, 4, 5, 6, 7, 8, 9 and 10. A range between 4 and 8 is suitable.

In some embodiments, viability of the population of cells is at least 50%.

In some embodiments, viability of the population of cells is at least 75%.

In some embodiments, viability of the population of cells is at least 85%.

In certain embodiments, viability of the population of cells is at least 95%.

In further embodiments, the population of cells comprises at least 1×10⁷ HSPCs cells.

In additional embodiments, the population of cells comprises at least 1×10⁸ HSPCs cells.

In certain embodiments, the population of cells comprises at least 1×10⁹ HSPCs cells.

Prior to, during and after the transduction the cells may be cultured in media suitable for the maintenance, growth, or proliferation of the cells. Suitable culture media and conditions are well known in the art. Such media include, for example, Dulbecco's Modified, Eagle's Medium® (DMEM), DMEM F12 Medium®, F-12K Medium®, Iscove's Modified Dulbecco's Medium®, Iscove's Minimal Essential Medium® (IMEM), RPMI-1640 Medium®, Stemline® II Hematopoietic Stem Cell Media (Merck), and serum-free medium for culture and expansion of haematopoietic cells SFEM®. Such media can then be supplemented with agents such as cytokines, PGE2 etc as discussed below and elsewhere herein.

Enhancing Transduction Efficacy

It is known that viral transduction efficacy can be enhanced by culturing the cells with various agents referred to herein as transduction enhancers (TEs).

In some embodiments, the cells are transduced by contacting the cells with a viral vector (e.g., a viral vector described above) and a substance that reduces activity and/or expression of protein kinase C (PKC). Such substance can be added as a supplement to the cell culture medium.

Suitably, the substance that reduces activity and/or expression of PKC is a PKC inhibitor like staurosporine or a variant thereof.

In some embodiments, the cell is contacted with stauprimide, e.g., as described in Caravatti et al. Bioorg. Med. Chem. Letters 4:199-404, 1994, the disclosure of which is hereby incorporated by reference in its entirety.

Transduction Using an HDAC Inhibitor

HDAC inhibitors have also been shown to increase transduction efficacy. A variety of agents can be used to inhibit histone deacetylases in order to increase the expression of a transgene during viral transduction. Without wishing to be bound by theory, reduced transgene expression from viral vectors may be caused by epigenetic silencing of vector genomes carried out by histone deacetylates. Hydroxamic acids represent a particularly robust class of HDAC inhibitors that inhibit these enzymes by virtue of hydroxamate functionality that binds cationic zinc within the active sites of these enzymes. Exemplary inhibitors include trichostatin A, as well as Vorinostat (N-hydroxy-N′-phenyl-octanediamide, described in Marks et al., Nature Biotechnology 25, 84 to 90 (2007); Stenger, Community Oncology 4, 384-386 (2007), the disclosures of which are incorporated by reference herein). Other HDAC inhibitors include Panobinostat, described in Drugs of the Future 32(4): 315-322 (2007), the disclosure of which is incorporated herein by reference.

Additional examples of hydroxamic acid inhibitors of histone deacetylases include the compounds shown below, described in Bertrand, European Journal of Medicinal Chemistry 45:2095-2116 (2010), the disclosure of which is incorporated herein by reference.

Other HDAC inhibitors that do not contain a hydroxamate substituent have also been developed, including Valproic acid (Gottlicher, et al., EMBO J. 20(24): 6969-6978 (2001) and Mocetinostat (N-(2-Aminophenyl)-4-[[(4-pyridin-3-ylpyrimidin-2-yl)amino]methyl]benzamide, described in Balasubramanian et al., Cancer Letters 280: 211-221 (2009)), the disclosure of each of which is incorporated herein by reference. Other small molecule inhibitors that exploit chemical functionality distinct from a hydroxamate include those described in Bertrand, European Journal of Medicinal Chemistry 45:2095-2116 (2010), the disclosure of which is incorporated herein by reference.

Additional examples of chemical modulators of histone acetylation useful with the compositions and methods of the invention include modulators of HDAC1, HDAC2, HDAC3, HDAC4, HDAC5, HDAC6, HDAC7, HDAC8, HDAC9, HDAC10, Sirt1, Sirt2, and/or HAT, such as butyrylhydroxamic acid, M344, LAQ824 (Dacinostat), AR-42, Belinostat (PXD101), CUDC-101, Scriptaid, Sodium Phenylbutyrate, Tasquinimod, Quisinostat (JNJ-26481585), Pracinostat (SB939), CUDC-907, Entinostat (MS-275), Mocetinostat (MGCD0103), Tubastatin A HCl, PCI-34051, Droxinostat, PCI-24781 (Abexinostat), RGFP966, Rocilinostat (ACY-1215), C1994 (Tacedinaline), Tubacin, RG2833 (RGFP109), Resminostat, Tubastatin A, BRD73954, BG45, 4SC-202, CAY10603, LMK-235, Nexturastat A, TMP269, HPOB, Cambinol, and Anacardic Acid.

In some particular embodiments, the HDAC inhibitor is Scriptaid.

Transduction Enhancement Using a Cyclosporine

In some embodiments, the modified HSPCs of the invention are produced by transducing the cells with the viral vector in the presence of a cyclosporine, such as cyclosporine A (CsA) or cyclosporine H (CsH). The cyclosporine can be added as a supplement to the cell culture medium.

In some embodiments, the concentration of the cyclosporine, when contacted with the cell, is from about 1 μM to about 10 μM (e.g., about 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.1 μM, 3.2 μM, 3.3 μM, 3.4 μM, 3.5 μM, 3.6 μM, 3.7 μM, 3.8 μM, 3.9 μM, 4 μM, 4.1 μM, 4.2 μM, 4.3 μM, 4.4 μM, 4.5 μM, 4.6 μM, 4.7 μM, 4.8 μM, 4.9 μM, 5 μM, 5.1 μM, 5.2 μM, 5.3 μM, 5.4 μM, 5.5 μM, 5.6 μM, 5.7 μM, 5.8 μM, 5.9 μM, 6 μM, 6.1 μM, 6.2 μM, 6.3 μM, 6.4 μM, 6.5 μM, 6.6 μM, 6.7 μM, 6.8 μM, 6.9 μM, 7 μM, 7.1 μM, 7.2 μM, 7.3 μM, 7.4 μM, 7.5 μM, 7.6 μM, 7.7 μM, 7.8 μM, 7.9 μM, 8 μM, 8.1 μM, 8.2 μM, 8.3 μM, 8.4 μM, 8.5 μM, 8.6 μM, 8.7 μM, 8.8 μM, 8.9 μM, 9 μM, 9.1 μM, 9.2 μM, 9.3 μM, 9.4 μM, 9.5 μM, 9.6 μM, 9.7 μM, 9.8 μM, 9.9 μM, or 10 μM).

Transduction Using an Activator of Prostaglandin E Receptor Signalling

Culturing of HSPCs in the presence of a compound that activated prostaglandin E receptor signalling, in particular prostaglandin E2 (PgE2) and dimethylprostaglandin E2 (dmPGE2) has been shown to increase the transduction efficacy of viral vectors, including lentiviral vectors.

In some embodiments, the modified HSPCs of the invention are produced by transducing the cells in the presence of an activator of prostaglandin E receptor signalling. Suitably, the activator of prostaglandin E receptor signalling is added as a supplement to the cell culture medium.

In some embodiments, the activator of prostaglandin E receptor signalling is a small molecule, such as a compound described in WO 2007/112084 or WO 2010/108028, the disclosures of each of which are incorporated herein by reference as they pertain to prostaglandin E receptor signalling activators.

In some embodiments, the activator of prostaglandin E receptor signalling is a small molecule, such as a small organic molecule, a prostaglandin, a Wnt pathway agonist, a cAMP/PI3K/AKT pathway agonist, a Ca²⁺ second messenger pathway agonist, a nitric oxide (NO)/angiotensin signalling agonist, or another compound known to stimulate the prostaglandin signalling pathway, such as a compound selected from Mebeverine, Flurandrenolide, Atenolol, Pindolol, Gaboxadol, Kynurenic Acid, Hydralazine, Thiabendazole, Bicuciline, Vesamicol, Peruvoside, Imipramine, Chlorpropamide, 1,5-Pentamethylenetetrazole, 4-Aminopyridine, Diazoxide, Benfotiamine, 12-Methoxydodecenoic acid, N-Formyl-Met-Leu-Phe, Gallamine, IAA 94, Chlorotrianisene, and or a derivative of any of these compounds.

In some embodiments, the activator of prostaglandin E receptor signalling is a naturally-occurring or synthetic chemical molecule or polypeptide that binds to and/or interacts with a prostaglandin E receptor, typically to activate or increase one or more of the downstream signalling pathways associated with a prostaglandin E receptor.

In some embodiments, the activator of prostaglandin E receptor signalling is selected from the group consisting of: prostaglandin A2 (PGA2), PGB2, PGD2, PGE1 (Alprostadil), PGE2, PGF2, PGI2 (Epoprostenol), PGH2, PGJ2, and derivatives and analogs thereof.

In some embodiments, the activator of prostaglandin E receptor signalling is PGE2 or dmPGE2.

In some embodiments, the activator of prostaglandin E receptor signalling is 15d-PGJ2, deltal2-PGJ2, 2-hydroxyheptadecatrienoic acid (HHT), Thromboxane (TXA2 and TXB2), PGI2 analogs, e.g., Iloprost and Treprostinil, PGF2 analogs, e.g., Travoprost, Carboprost tromethamine, Tafluprost, Latanoprost, Bimatoprost, Unoprostone isopropyl, Cloprostenol, Oestrophan, and Superphan, PGE1 analogs, e.g., 11-deoxy PGE1, Misoprostol, and Butaprost, and Corey alcohol-A ([3aa,4a,5,6aa]-(−)-[Hexahydro-4-(hydroxymetyl)-2-oxo-2H-cyclopenta/b/furan-5-yl][1,1-biphenyl]-4-carboxylate), Corey alcohol-B (2H-Cyclopenta[b]furan-2-on,5-(benzoyloxy)hexahydro-4-(hydroxymethyl)[3aR-(3aa,4a,5,6aa)]), and Corey diol ((3aR,4S,5R,6aS)-hexahydro-5-hydroxy-4-(hydroxymethyl)-2H-cyclopenta[b]furan-2-one).

In some embodiments, the activator of prostaglandin E receptor signalling is a prostaglandin E receptor ligand, such as prostaglandin E2 (PGE2), or an analogs or derivative thereof. Prostaglandins refer generally to hormone-like molecules that are derived from fatty acids containing 20 carbon atoms, including a 5-carbon ring, as described herein and known in the art. Illustrative examples of PGE2 “analogs” or “derivatives” include, but are not limited to, 16,16-dimethyl PGE2, 16-16 dimethyl PGE2 p-(p-acetamidobenzamido) phenyl ester, I I-deoxy-16,16-dimethyl PGE2, 9-deoxy-9-methylene-16, 16-dimethyl PGE2, 9-deoxy-9-methylene PGE2, 9-keto Fluprostenol, 5-trans PGE2, 17-phenyl-omega-trinor PGE2, PGE2 serinol amide, PGE2 methyl ester, 16-phenyl tetranor PGE2, 15(S)-15-methyl PGE2, 15 (R)-15-methyl PGE2, 8-iso-15-keto PGE2, 8-iso PGE2 isopropyl ester, 20-hydroxy PGE2, nocloprost, sulprostone, butaprost, 15-keto PGE2, and 19 (R) hydroxy PGE2.

In some embodiments, the activator of prostaglandin E receptor signalling is a prostaglandin analog or derivative having a similar structure to PGE2 that is substituted with halogen at the 9-position (see, e.g., WO 2001/12596, herein incorporated by reference in its entirety), as well as 2-decarboxy-2-phosphinico prostaglandin derivatives, such as those described in US 2006/0247214, herein incorporated by reference in its entirety).

In some embodiments, the activator of prostaglandin E receptor signalling is a non-PGE2-based ligand. In some embodiments, the activator of prostaglandin E receptor signalling is CAY10399, ONO_8815Ly, ONO-AE1-259, or CP-533,536. Additional examples of non-PGE2-based EP2 agonists include the carbazoles and fluorenes disclosed in WO 2007/071456, herein incorporated by reference for its disclosure of such agents. Illustrative examples of non-PGE2-based EP₃ agonist include, but are not limited to, AE5-599, MB28767, GR 63799X, ONO-NT012, and ONO-AE-248. Illustrative examples of non-PGE₂-based EP₄ agonist include, but are not limited to, ONO-4819, APS-999 Na, AH23848, and ONO-AE 1-329. Additional examples of non-PGE2-based EP4 agonists can be found in WO 2000/038663; U.S. Pat. Nos. 6,747,037; and 6,610,719, each of which are incorporated by reference for their disclosure of such agonists

In some embodiments, the activator of prostaglandin E receptor signalling is a Wnt agonist. Illustrative examples of Wnt agonists include, but are not limited to, Wnt polypeptides and glycogen synthase kinase 3 (GSK3) inhibitors. Illustrative examples of Wnt polypeptides suitable for use as compounds that stimulate the prostaglandin EP receptor signalling pathway include, but are not limited to, Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt1Oa, Wnt1Ob, Wnt11, Wnt14, Wnt15, or biologically active fragments thereof. GSK3 inhibitors suitable for use as agents that stimulate the prostaglandin EP receptor signalling pathway bind to and decrease the activity of GSK3a, or GSK3. Illustrative examples of GSK3 inhibitors include, but are not limited to, BIO (6-bromoindirubin-3′-oxime), LiCl, Li₂CO₃, or other GSK-3 inhibitors, as exemplified in U.S. Pat. Nos. 6,057,117 and 6,608,063, as well as US 2004/0092535 and US 2004/0209878, and ATP-competitive, selective GSK-3 inhibitors CHIR-911 and CHIR-837. (also referred to as CT-99021/CHIR-99021 and CT-98023/CHIR-98023, respectively) (Chiron Corporation (Emeryville, Calif.)).

In some embodiments, method further includes contacting the cell with a GSK3 inhibitor. Suitably, the GSK3 inhibitor can be added to the cell culture medium.

In some embodiments, the activator of prostaglandin E receptor signalling is an agent that increases signalling through the cAMP/P13K/AKT second messenger pathway, such as an agent selected from the group consisting of dibutyryl cAMP (DBcAMP), phorbol ester, forskolin, sclareline, 8-bromo-cAMP, cholera toxin (CTx), aminophylline, 2,4 dinitrophenol (DNP), norepinephrine, epinephrine, isoproterenol, isobutylmethylxanthine (IBMX), caffeine, theophylline (dimethylxanthine), dopamine, rolipram, iloprost, pituitary adenylate cyclase activating polypeptide (PACAP), and vasoactive intestinal polypeptide (VIP), and derivatives of these agents.

In some embodiments, the activator of prostaglandin E receptor signalling is an agent that increases signalling through the Ca²⁺ second messenger pathway, such as an agent selected from the group consisting of Bapta-AM, Fendiline, Nicardipine, and derivatives of these agents.

In some embodiments, the activator of prostaglandin E receptor signalling is an agent that increases signalling through the NO/Angiotensin signalling, such as an agent selected from the group consisting of L-Arg, Sodium Nitroprusside, Sodium Vanadate, Bradykinin, and derivatives thereof.

Transduction Using a Polycationic Polymer

In some embodiments, therapeutic cells of the disclosure are produced by transducing the cells in the presence of a polycationic polymer. Suitably, the polycationic polymer can be added to the cell culture medium.

In some embodiments, the polycationic polymer is polybrene, protamine sulfate, polyethylenimine, or a polyethylene glycol/poly-L-lysine block copolymer.

In some embodiments, the polycationic polymer is protamine sulfate.

In some embodiments, the cell is further contacted with an expansion agent during the transduction procedure. The cell may be, for example, a hematopoietic stem cell and the expansion agent may be a hematopoietic stem cell expansion agent, such as a hematopoietic stem cell expansion agent known in the art or described herein.

Additional Transduction Enhancers

In some embodiments of the methods described herein, during the transduction procedure, the cell is further contacted with an agent that inhibits mTOR signalling. The agent that inhibits mTOR signalling may be, for example, rapamycin, among other suppressors of mTOR signalling.

Additional transduction enhancers that may be used in conjunction with the compositions and methods of the disclosure include, for example, tacrolimus and vectorfusin.

Spinoculation

In some embodiments of the disclosure, a cell or population of cells targeted for transduction may be spun e.g., by centrifugation, while being cultured with a viral vector (e.g., in combination with one or more additional agents described herein). This “spinoculation” process may occur with a centripetal force of, e.g., from about 200×g to about 2,000×g. The centripetal force may be, e.g., from about 300×g to about 1,200×g (e.g., about 300×g, 400×g, 500×g, 600×g, 700×g, 800×g, 900×g, 1,000×g, 1,100×g, or 1,200×g, or more). In some embodiments, the cell is spun for from about 10 minutes to about 3 hours (e.g., about 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, or more). In some embodiments, the cell is spun at room temperature, such as at a temperature of about 25° C.

Exemplary transduction protocols involving a spinoculation step are described, e.g., in Millington et al., PLoS One 4:e6461 (2009); Guo et al., Journal of Virology 85:9824-9833 (2011); O'Doherty et al., Journal of Virology 74:10074-10080 (2000); and Federico et al., Lentiviral Vectors and Exosomes as Gene and Protein Delivery Tools, Methods in Molecular Biology 1448, Chapter 4 (2016), the disclosures of each of which are incorporated herein by reference.

Pre-Treatment Conditioning

The modified HSPCs that express a progranulin transgene according to the present invention can be administered to a patient in need thereof as described herein. In such instances, prior to administration of the cells to the patient, the patient may be administered an agent that ablates an endogenous population of CD34⁺ cells, allowing the administered CD34⁺ cells to engraft in the patient. Examples of conditioning agents include myeloablative conditioning agents, which deplete a wide variety of hematopoietic cells in a patient. For instance, that patient may be pre-treated with an alkylating agent, such as a nitrogen mustard (e.g., bendamustine, chlorambucil, cyclophosphamide, ifosfamide, mechlorethamine, or melphalan), a nitrosourea (e.g., carmustine, lomustine, or streptozocin), an alkyl sulfonate (e.g., busulfan), a triazine (e.g., dacarbazine or temozolomide), or an ethylenimine (e.g., altretamine or thiotepa). In some embodiments, the patient is administered a conditioning agent that selectively ablates a specific population of endogenous cells, such as a population of endogenous CD34⁺ HSCs or HPCs.

Methods of Measuring Progranulin Gene Expression

Preferably, the compositions and methods of the disclosure are used to facilitate expression of functional progranulin at physiologically normal levels in a patient or therapeutically beneficial levels (e.g., in a human patient having FTD). In some embodiments, the introduced modified HSPCs of the invention may effect progranulin expression in a FTD patient at a level of, for example, from about 20% to about 200% of the level of functional progranulin expression observed in a human subject of comparable age and body mass index that does not have a progranulin deficiency (e.g., about 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% of the level of functional progranulin expression observed in a human subject of comparable age and body mass index that does not have a progranulin deficiency).

Introduction of the modified HSPCs of the invention may, for example, effectuate expression of functional progranulin in the desired location or type of cells (e.g. microglia) at a level of about 20% of that observed in a human subject of comparable age and body mass index that does not have FTD. In some embodiments, it effectuates expression of functional progranulin at a level of about 30% of that observed in a human subject of comparable age and body mass index that does not have FTD. In some embodiments, it effectuates expression of functional progranulin at a level of about 40% of that observed in a human subject of comparable age and body mass index that does not have FTD. In some embodiments, it effectuates expression of functional progranulin at a level of about 50% of that observed in a human subject of comparable age and body mass index that does not have FTD. In some embodiments, it effectuates expression of functional progranulin at a level of about 60% of that observed in a human subject of comparable age and body mass index that does not have FTD. In some embodiments, it effectuates expression of functional progranulin at a level of about 70% of that observed in a human subject of comparable age and body mass index that does not have FTD. In some embodiments, it effectuates expression of functional progranulin at a level of about 80% of that observed in a human subject of comparable age and body mass index that does not have FTD. In some embodiments, it effectuates expression of functional progranulin at a level of about 90% of that observed in a human subject of comparable age and body mass index that does not have FTD. In some embodiments, it effectuates expression of functional progranulin at a level of about 100% of that observed in a human subject of comparable age and body mass index that does not have FTD. In some embodiments, it effectuates expression of functional progranulin at a level of about 110% of that observed in a human subject of comparable age and body mass index that does not have FTD. In some embodiments, it effectuates expression of functional progranulin at a level of about 120% of that observed in a human subject of comparable age and body mass index that does not have FTD. In some embodiments, it effectuates expression of functional progranulin at a level of about 130% of that observed in a human subject of comparable age and body mass index that does not have FTD. In some embodiments, it effectuates expression of functional progranulin at a level of about 140% of that observed in a human subject of comparable age and body mass index that does not have FTD. In some embodiments, it effectuates expression of functional progranulin at a level of about 150% of that observed in a human subject of comparable age and body mass index that does not have FTD. In some embodiments, it effectuates expression of functional progranulin at a level of about 160% of that observed in a human subject of comparable age and body mass index that does not have FTD. In some embodiments, it effectuates expression of functional progranulin at a level of about 170% of that observed in a human subject of comparable age and body mass index that does not have FTD. In some embodiments, it effectuates expression of functional progranulin at a level of about 180% of that observed in a human subject of comparable age and body mass index that does not have FTD. In some embodiments, it effectuates expression of functional progranulin at a level of about 190% of that observed in a human subject of comparable age and body mass index that does not have FTD. In some embodiments, it effectuates expression of functional progranulin at a level of about 200% of that observed in a human subject of comparable age and body mass index that does not have FTD.

The expression level of functional progranulin expressed in the desired cells or tissues of a patient can be ascertained, for example, by evaluating the concentration or relative abundance of mRNA transcripts derived from transcription of a functional progranulin transgene. Additionally, or alternatively, gene expression can be determined by evaluating the concentration or relative abundance of functional progranulin protein produced by transcription and translation of a progranulin transgene. Protein concentrations can also be assessed using functional assays, such as MDP detection assays. The sections that follow describe exemplary techniques that can be used to measure the expression level of a progranulin transgene upon delivery to a patient, such as a patient having FTD as described herein. Transgene expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

Nucleic Acid Detection

Nucleic acid-based methods for determining progranulin transgene expression detection that may be used in conjunction with the compositions and methods described herein include imaging-based techniques (e.g., Northern blotting or Southern blotting). Such techniques may be performed using cells obtained from a patient following administration of the progranulin transgene. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, N.Y. 11803-2500. Typical protocols for evaluating the status of genes and gene products are found, for example in Ausubel et al., eds., 1995, Current Protocols in Molecular Biology, Units 2 (Northern Blotting), 4 (Southern Blotting), 15 (Immunoblotting) and 18 (PCR Analysis).

Transgene detection techniques that may be used in conjunction with the compositions and methods described herein to evaluate progranulin expression further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For instance, transgene expression at the mRNA level may be determined using RNA-Seq (e.g., as described in Mortazavi et al., Nat. Methods 5:621-628 (2008) the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Transgene expression levels may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labelled to generate cDNA. The probes can then hybridize to one or more complementary nucleic acids arrayed and immobilized on a solid support. The array can be configured, for example, such that the sequence and position of each member of the array is known. Hybridization of a labelled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labelled target from RNA isolated from the sample, 2) hybridization of the labelled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of a transgene in a target cell following delivery to a patient. In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

Protein Detection

Transgene expression can additionally be determined by measuring the concentration or relative abundance of a corresponding protein product (e.g., progranulin) encoded by a gene of interest. Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize transgene expression in a cell from a patient (e.g., a human patient) following delivery of the transgene encoding progranulin. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

Freeze Thawing of Modified HSPCs.

Suitably, after the HSPCs have been modified according to the present invention they can be used directly for treating a patient or they can be cryopreserved for later use following appropriate thawing.

Methods and formulations for cryopreserving and then thawing HSPCs for subsequent use are known (see for example, WO2018/046457, incorporated herein by reference).

Suitably the modified HSPCs are formulated in a cryoprotective formulation. Suitably, the cryoprotective formulation comprises about 5% by volume of dimethyl sulfoxide (DMSO) and about 7% weight by volume of human serum albumin (HSA). The modified HSPCs are suspended in the cryoprotective formulation and then subjected to freezing.

The modified HSPCs are frozen at an appropriate cell concentration, such as a concentration of at least about 1×10⁶/ml, such as at least 2×10⁶/ml, 5×10⁶/ml or 10×10⁶/ml (i.e. 1×10⁷/ml).

There are many standard methods known in the art which can be used to freeze the cells, e.g. immersing containers holding the cell suspension in a solid carbon dioxide and alcohol mixture, or in liquid nitrogen, or placed directly in a freezer set at a desired temperature. In one embodiment, the suspension is frozen using a programmed freezer (i.e. controlled rate freezer). Controlled rate freezers are commercially available and well known in the art, for example the EF600M controlled rate freezer (Aysmptote). Such controlled rate freezers can be used to both freeze and thaw a suspension.

Suitably, the suspension is frozen at a temperature from about −200° C. to a temperature of about −35° C. such as about −80° C.

The cells may then be stored frozen for a significant period of time. Suitably for the viability of the modified HSPCs are maintained for at least 2 months, such as at least 4 months, at least 6 months and in particular at least 12 months.

There are many standard methods known in the art which can be used to thaw the frozen suspension, e.g. by allowing the suspension to thaw slowly at room temperature, or by immersing the frozen suspension in a liquid, e.g. a water-bath set at a temperature of about 37° C. Cells can also be thawed by mixing the suspension with a thawed medium. In one embodiment, the frozen suspension is thawed using a programmed freezer (e.g. a controlled rate freezer).

Prior to administration the cryoprotective formulation can be exchanged for a formulation suitable for delivery to the patient, as described herein.

Pharmaceutical Compositions and Dosing

In a further aspect, the disclosure provides a pharmaceutical composition comprising (i) a population of modified HSPCs as described herein that express functional progranulin and (ii) one or more carriers, diluents, and/or excipients. The cells (e.g., HSPCs) may be, for example, human cells, such as human HSCs and/or HPCs.

Exemplary carriers, diluents, and excipients that may be used in conjunction with the compositions and methods of the disclosure are described, e.g., in Remington: The Science and Practice of Pharmacy (2012, 22nd ed.) and in The United States Pharmacopeia: The National Formulary (2015, USP 38 NF 33).

In some embodiments, the composition is formulated for administration to a human patient. For example, the composition may be formulated for intravenous (IV) injection, intracerebroventricular (ICV) injection or intrathecal lumbar (ITL) injection to the human patient.

The modified HSPCs as described herein can be used in therapy.

The modified HSPCs as described herein can be administered as therapeutic compositions (e.g., as pharmaceutical compositions). Cellular compositions as described herein can be provided as sterile liquid preparations, e.g., isotonic aqueous solutions, suspensions, emulsions, dispersions, or viscous compositions, which may be buffered to a selected pH. A liquid preparation may be easier to prepare than a gel, another viscous composition, and a solid composition. Additionally, a liquid composition may be more convenient to administer (i.e., by injection). Viscous compositions, on the other hand, can be formulated within the appropriate viscosity range to provide longer contact periods with specific tissues. Liquid or viscous compositions can comprise a carrier, which can be a solvent or dispersing medium comprising, for example, water, saline, phosphate buffered saline, polyol (e.g., glycerol, propylene glycol, liquid polyethylene glycol, and the like), and suitable mixtures thereof.

Sterile injectable solutions can be prepared by incorporating the cells described herein in a sufficient amount of an appropriate diluent. Such compositions may be in admixture with a suitable carrier or excipient such as sterile water, physiological saline, glucose, dextrose, or another carrier or excipient suitable for delivering live cells to a subject. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like,depending upon the route of administration and the preparation desired. Standard texts, such as “Remington's Pharmaceutical Science”, 17th edition, 1985, incorporated herein by reference, may be consulted to prepare suitable preparations, without undue experimentation. Additives that enhance the stability and sterility of the cellular compositions, including antimicrobial preservatives, antioxidants, chelating agents, and buffers, can be added.

Prevention of the action of microorganisms can be ensured by an antibacterial or antifungal agent including, but not limited to, parabens, chlorobutanol, phenol, and sorbic acid. According to the present disclosure, however, any vehicle, diluent, or additive used must be compatible with the cells.

The compositions can be isotonic, i.e., they have the same osmotic pressure as blood and cerebrospinal fluid. The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride may be suitable for buffers containing sodium ions. Viscosity of the compositions, if desired, can be maintained at a selected level using a pharmaceutically acceptable thickening agent. In some embodiments, the thickening agent is methylcellulose, which is readily and economically available and is easy to work with. Other suitable thickening agents include, but are not limited to, xanthan gum, carboxymethyl cellulose, hydroxypropyl cellulose, and carbomer. The concentration of the thickener will depend upon the agent selected and the amount of the agent used. Suitable carriers and other additives maybe chosen depending on the route of administration and the nature of the dosage form (e.g., a liquid dosage form can be formulated into a solution, a suspension, a gel, or another liquid form, such as a time release formulation or liquid-filled form). An effective number/amount of cells to be administered can vary for the subject being treated. In one embodiment, between about 10⁴ to about 10⁸ cells, and in another embodiment between about 10⁵ to about 10⁷ cells are administered to a subject.

The skilled artisan can readily determine the amounts of cells and optional additives, vehicles, and/or carrier in compositions to be administered. In one embodiment any additive (in addition to the cell(s)) is present in an amount of about 0.001% to about 50% (weight) solution in phosphate buffered saline, and the active ingredient is present in the order of micrograms to milligrams, such as about 0.0001% to about 5 wt %. In another embodiment, the active ingredient is present at about 0.0001% to about 1 wt %. In yet another embodiment, the active ingredient is present at about 0.0001% to about 0.05 wt %. In still other embodiments, the active ingredient is present at about 0.001% to about 20 wt %. In some embodiments, the active ingredient is present at about 0.01% to about 10 wt %. In another embodiment, the active ingredient is present at about 0.05% to about 5 wt %. For any composition to be administered to an animal or human, and for any particular method of administration, toxicity can be determined by measuring the lethal dose (LD) and LD₅₀ in a suitable animal model e.g., a rodent such as mouse. The dosage of the composition(s), concentration of components therein, and timing of administering the composition(s), which elicit a suitable response can also be determined. Such determinations do not require undue experimentation in light of the knowledge of the skilled artisan, this disclosure, and the documents cited herein. The time for sequential administrations can also be ascertained without undue experimentation.

The compositions described herein may be administered to a patient with a neurodegenerative disease (e.g., a human patient suffering from FTD) by one or more of a variety of routes, such as intravenously (IV) or by intracerebroventricular (ICV) injection. In some embodiments, the patient has a loss-of-function mutation in the endogenous gene encoding progranulin. In some embodiments, the mutation is heterozygous. In some embodiments, the mutation is homozygous. The mutation may be, for example, c.1477C>T (p.Arg493Ter), c.26C>A (p.Ala9Asp), c.-8+5G>C. In some embodiments the mutation is a frameshift mutation, such as p.C31LfsX32, p.S82VfsX174, P.L271LfsX174 or p.T382NfsX32. In some embodiments the mutation is a frameshift, insertion, deletion or transversion mutation.

Numerous nonsense mutations, deletions and splice-site mutations have been detected in patients with FTD and CLN11. For example, Yu et al. (Arch Neurol 67(2):161-170, 2010, tested 434 patients with FTD. They sequenced all 13 GRN exons and at least 80 base pairs of flanking introns. They identified 58 genetic variants. Twenty-four of these appeared to be pathogenic.

Haploinsufficiency of GRN is the predominant mechanism leading to FTD See also Petkau and Leavitt, Trends Neurosci. 37(7):388-98, 2014, Kao et al., Nat Rev Neurosci. 18(6):325-333, 2017).

Other suitable examples of FTD pathogenic mutations are: p.A9D, p.V77I, p.C105R, p.T182M, p.T251S, p.A276V, p.R298H, p.R212W, p.P357R, p.R19W, p.G70S, and p.R433W

Homozygous GRN mutations have also been reported, although carriers present a vastly different clinical phenotype known as neuronal ceroid lipofuscinosis (NCL).

The number of modified HSPCs administered to a patient and the route of administration may depend on a number of factors, for example, on the expression level of the desired protein(s) in the cells, the percentage of successfully transduced cells, the vector copy number (VCN), the patient, pharmaceutical formulation methods, the speed needed to elicit a therapeutic effect, administration methods (e.g., administration time and administration route), the patient's age, body weight, sex, severity of the disease being treated, and whether or not the patient has been treated with agents to ablate endogenous pluripotent cells (e.g., endogenous CD34+ cells, hematopoietic stem or progenitor cells, or microglia, among others). The number of cells administered may be, for example, from 1×10⁶ cells/kg to 1×10¹² cells/kg, or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more). Cells may be administered in an undifferentiated state, or after partial or complete differentiation into microglia. The number of pluripotent cells may be administered in any suitable dosage form.

In some embodiments, the cell or the composition comprising the cell is administered to a subject in a targeted manner. For example, in some embodiments, a composition comprising a cell expressing progranulin is administered directly to a subject's brain. In some embodiments, the composition is delivered directly to the brain via intracerebroventricular (ICV) administration. In some embodiments, the composition is delivered in this manner to the lateral ventricles of the subject's brain.

ICV delivery following ablative conditioning is superior to conventional methods for HSC transplantation in timing and extent of replacement of resident microglia by the progeny of the transplanted cells and allows exclusive transplanted cell engraftment in the CNS. Conventional methods for HSC transplantation include the use of total bone marrow or aphaeretic products or cord blood, or of hematopoietic stem and progenitor cells (HSPCs) in the case of autologous gene therapy. In contrast, the present invention provides cell populations enriched for cells having microglia-repopulating activity. The ICV approach is therapeutically beneficial as this delivers the modified HSPCs directly to the brain which improves the speed and extent of microglia reconstitution by the transplanted donor cells and increases the therapeutic protein delivery to the brain as compared toa single intravenous (IV) transplantation.

In a particular embodiment, the modified HSPCs of the present invention are delivered directly to the brain after ablative conditioning.

Alternative intra-CNS routes include ITL that enhances engraftment of the transplanted cells and generate a myeloid/microglia progeny, but also favors bone marrow/hematopoietic engraftment of the transplanted cells.

Alternatively, the composition may be delivered systemically, such as by intravenous administration. Cells administered in such a manner must traverse the blood brain barrier prior to engrafting in the subject's brain. Other modes of administration (parenteral, mucosal, implant, intraperitoneal, intradermal, transdermal, intramuscular, intravenous including infusion and/or bolus injection, and subcutaneous) are generally known in the art. In some embodiments, cells are administered in a medium suitable for injection, such as phosphate buffered saline, into a subject. Because the cells being administered to the subject are intended to repopulate microglia cells, intracerebroventricular administration may be advantageous as other routes of administration require crossing the blood brain barrier. Engraftment of transplanted cells into a subject's brain provides a population of cells that express a therapeutic agent. But because the transplanted cells are meant to replace endogenous cells (i.e., microglia cells), in certain embodiments, methods of treating a subject having susceptibility to, or at risk of developing a neurodegenerative disease, such as FTD, further comprise administering to a subject prior to administering the modified HSPCs of the invention, an agent for ablating endogenous microglia and/or their progenitors. In some embodiments, the agent is an alkylating agent. In particular, nanoparticle delivery of alkylating agents may be effective in creating a suitable environment for engraftment of transplanted HSPCs, as described in WO2018/071898, the contents of which is incorporated herein by reference in its entirety. Multiple routes of administration may be used to treat a single patient at one time, or the patient may receive treatment via one route of administration first and receive treatment via another route of administration during a second appointment, e.g., 1 week later, 2 weeks later, 1 month later, 6 months later, or 1 year later. Compositions may be administered to a subject once, or cells may be administered one or more times (e.g., 2-10 times) per week, month, or year.

Diagnosis

The modified HSPCs and methods of the invention are particularly suited for use in the prevention or treatment of a patient with or susceptible to developing a neurodegenerative disease mediated by mutant endogenous progranulin, such as FTD. A patient (e.g., a human patient) can be diagnosed as having FTD in a variety of ways. Genetic testing offers one avenue by which a patient may be diagnosed as having (or at risk of developing) FTD. For example, a genetic analysis can be used to determine whether a patient has a loss-of-function mutation in the endogenous gene encoding progranulin, such as a mutation in a progranulin gene selected from the group consisting of: c.1477C>T (p.Arg493Ter), c.26C>A (p.Ala9Asp), c.-8+5G>C. Exemplary genetic tests that can be used to determine whether a patient has such a mutation include polymerase chain reaction (PCR) methods known in the art and described herein, among others.

Clinically, FTD may be detected, for example, by way of clinical presentations that resemble behavioural variant FTD (such as early bahavioral disinhibition, early apathy, compulsive/ritualistic behaviour), primary progressive aphasia (difficulty with language) and atypical parkinsonism. The clinical diagnosis can be combined with neuroimaging (Computed Tomography, Magnetic Resonance Imaging, Positron Emission Tomography), which may reveal brain atrophy, decreased perfusion and glucose metabolism in the frontal and temporal lobes. The diagnosis for GRN-FTD is established by molecular genetic testing.

Prevention

Using the compositions and methods described herein, a subject (e.g., a human subject) may be administered the modified HSPCs so as to prevent the onset of FTD. The subject may be one that is at risk of developing FTD but has not yet presented with an observable symptom of the disease. For example, the subject may be one that has a loss-of-function mutation in the endogenous gene encoding progranulin, such as a mutation in a progranulin gene selected from the group consisting of: c.1477C>T (p.Arg493Ter), c.26C>A (p.Ala9Asp), c.-8+5G>C. As described above, a subject can be identified as having such a mutation using standard molecular biology techniques known in the art and described herein, including PCR-based methodologies, among others.

Method of Treatment

The population of HSPCs modified to express functional progranulin as described herein, or a pharmaceutical composition comprising such population of cells can be used to treat or prevent a patient from developing a neurodegenerative disease mediated by defective expression of progranulin, which included null expression, subnormal expression levels of expression of a dysfunctional variant of progranulin. Such defective expression is often manifest by mutation in the GRN gene. The presence of defective progranulin expression and/or a pathogenic mutation in the GRN gene, can be determined as described herein.

The modified HSPCs contribute to the turnover of brain resident myeloid populations upon transplantation in recipients receiving a proper pre-transplant preparatory regimen. Transplanted HSPCs home to the brain, engraft locally and give rise to a mature progeny that shares transcriptional, morphologic and functional features with microglia when functionally defined microglia progenitors are (partially) ablated by pre-transplant chemotherapy.

According to another aspect of the invention there is provided the HSPCs of the first aspect of the invention or the HSPC produced by the second aspect if the invention or a composition of the third aspect of the invention for use in therapy.

According to the fourth aspect of the invention there is provided a method of treating a neurodegenerative disorder mediated by aberrant expression of progranulin, such as frontotemporal dementia (FTD) or neuronal ceroid lipofuscinosis (NCL), in a subject in need thereof, the method comprising administering to the subject the HSPCs of the first aspect of the invention or the HSPC produced by the second aspect if the invention or a composition of the third aspect of the invention.

According to the fifth aspect of the invention there is provided a population of progranulin expressing HSPCs of the first aspect of the invention or the HSPCs produced by the second aspect of the invention or the composition of the third aspect of the invention for use in the prevention or treatment of a neurodegenerative disorder mediated by aberrant expression of progranulin such as frontotemporal dementia (FTD) or neuronal ceroid lipofuscinosis (NCL).

According to the sixth aspect of the invention there is provided a method of preventing or treating a disease mediated by a dysfunctional gene encoding progranulin, the method comprising:
(i) determining whether a subject has a mutation in the progranulin gene that leads to aberrant expression or amount of progranulin; and,
(ii) if the subject has a mutation in the progranulin gene that leads to aberrant expression or amount of progranulin they are administered the HSPCs of the first aspect of the invention or the HSPC produced by the second aspect if the invention or a composition of the third aspect of the invention.

According to a variant of the sixth aspect of the invention there is provided a population of modified HSPCs of the first aspect of the invention or the HSPC produced by the second aspect if the invention or a composition of the third aspect of the invention for use in the prevention or treatment of a disease mediated by a dysfunctional gene encoding progranulin, the method comprising:
(i) determining whether a subject has a mutation in the progranulin gene that leads to aberrant expression or amount of progranulin; and,
(ii) if the subject has a mutation in the progranulin gene that leads to aberrant expression or amount of progranulin they are administered

In particular embodiments, determining whether a subject has a mutation in the progranulin gene that leads to aberrant expression or amount of progranulin is carried out on a sample previously isolated from the subject. Suitably the sample comprises peripheral blood or other body tissue.

In embodiments of the fourth, fifth or sixth aspects the neurodegenerative disorder mediated by aberrant expression of progranulin is selected from the group consisting of: frontotemporal dementia (FTD), neuronal ceroid lipofuscinosis (NCL), amyotrophic lateral sclerosis (ALS), Alzheimer's disease (AD) and Parkinson's disease (PD).

In embodiments of the fourth, fifth or sixth aspects the neurodegenerative disorder mediated by aberrant expression of progranulin is frontotemporal dementia (FTD).

In particular embodiments, when the modified HSPCs comprise progranulin under the control of a regulated promoter that is activated by cytokines, such as for example, DRA-HLA promoter, such cells produce an enhanced level of progranulin when an activating cytokine is present, such as for example interferon gamma. Such activating cytokines are more prevalent in inflammatory diseased states and so expression of the anti-inflammatory progranulin is regulated by inflammatory signals.

Therapeutic Activities of Agents that Increase Progranulin Expression

A health care professional may diagnose a subject as having a neurodegenerative disease mediated by a dysfunctional progranulin gene using standard molecular biological diagnostic tools. Numerous nonsense mutations, deletions and splice-site mutations have been detected in patients with FTD and CLN11. For example, Yu et al. (Arch Neurol 67(2):161-170, 2010, tested 434 patients with FTD. They sequenced all 13 GRN exons and at least 80 base pairs of flanking introns. Thy identified 58 genetic variants. Twenty-four of these appeared to be pathogenic. Haploinsufficiency of GRN is the predominant mechanism leading to FTD.

Examples of FTD pathogenic mutations are: p.A9D, p.V77I, p.C105R, p.T182M, p.T251S, p.A276V, p.R298H, p.R212W, p.P357R, p.R19W, p.G70S, and p.R433W.

In some embodiments, after providing the patient with the modified HSPCs that express functional progranulin, the patient exhibits sustained disease remission. For example, the patient may exhibit sustained disease remission for 30 days, two months, three months, four months, five months, six months, seven months, eight months, nine months, ten months, eleven months, or one year or longer after providing the patient with the modified HSPCs that express functional progranulin.

In some embodiments, after providing the patient with the modified HSPCs that express functional progranulin, the patient exhibits enhancements over baseline neuropsychological and neurophysiological evaluation, including assessment of short interval intracortical inhibition (SICI), intracortical facilitation (ICF), short interval intracortical facilitation (SICF) and long interval intracortical inhibition (LICI), as assessed using transcranial magnetic stimulation (TMS), according to standard protocols.

Other positive signal outcome measures could be, or be based on, any of the following neuropsychological tests:
Change in Mini Mental State Examination (MMMSE) scores from baseline

(MMMSE is questionnaire that is used to measure cognitive impairment);
Change in phonemic fluencies scores from baseline (e.g. produce as many words as possible beginning with a specified letter in 60 seconds);
Change in semantic fluencies scores from baseline (e.g. produce as many words as possible from a category in 60 seconds);
Change in digit span forward scores from baseline (e.g. participants hear a sequence of numerical digits and are tasked to recall the sequence correctly, with increasingly longer sequences being tested in each trial. The participant's span is the longest number of sequential digits that can accurately be remembered;
Change in digit span backward scores from baseline (e.g. participants hear a sequence of numerical digits and are tasked to recall the sequence correctly in reverse order, with increasingly longer sequences being tested in each trial. The participant's span is the longest number of sequential digits that can accurately be remembered;
Change in camel and cactus test scores from baseline (e.g. evaluates associative semantic memory with 64 items presented for naming and word-picture matching);
Change in Trail Making Test A (TMTA) scores from baseline (e.g. a task that requires a subject to connect a sequence of 25 consecutive targets on a sheet of paper to examine cognitive processing speed;
Change in Trail Making Test B (TMTB) scores from (e.g. a task that requires a subject to connect a sequence of 25 consecutive targets on a sheet of paper, alternating between numbers and letters, to examine executive functioning;
Change in Stroop test scores from baseline (measure a person's selective attention capacity and skills, as well as their processing speed ability;
Change in Symbol Digit test scores from baseline (It consists of digit-symbol pairs followed by a list of digits. Under each digit the subject should write down the corresponding symbol as fast as possible. The number of correct symbols within the allowed time is measured;
Change in Block Design test scores from baseline (evaluates spatial visualization ability and motor skills. The test-taker uses hand movements to rearrange blocks that have various color patterns on different sides to match a pattern. The items in a block design test are scored both by accuracy in matching the pattern and by speed in completing each item;
Change in The modified EkmanFaces Test from baseline (e.g. each face is presented on a sheet with six labels of basic emotions below the photograph. The patient is required to respond verbally, deciding the label that best described the facial expression shown).
Quality of life questionnaire

The tests are carried out at appropriate intervals from therapeutic intervention. Such as 2, 4, 12, 26 and 52 week intervals from baseline.

In some embodiments, after providing the patient with the modified HSPCs that express functional progranulin, the patient no longer requires treatment with Antidepressant/antipsychotic. For example, the patient may not require drug treatment for at least three months (e.g., for from about three months to about one year, such as for three months, four months, five months, six months, seven months, eight months, nine months, ten months, eleven months, or one year, or longer). In some embodiments, the patient does not require treatment with the immunosuppressive agents, biologic agents, and/or corticosteroids for up to five years.

In some embodiments, after providing the patient with the modified HSPCs that express functional progranulin, the patient exhibits an improvement in CSF progranulin levels.

For example, after providing the patient with the modified HSPCs that express functional progranulin, the patient may not exhibit evidence of microglia activation for at least three months (e.g., for from about three months to about one year, such as for three months, four months, five months, six months, seven months, eight months, nine months, ten months, eleven months, or one year, or longer). In some embodiments, the patient does not exhibit evidence of microglia activation by positron emission tomography (PET) for up to five years.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991).

### PARTICULAR EMBODIMENTS OF THE INVENTION

E1. A population of modified hematopoietic stem cells (HSPCs) where the cells carry an exogenous copy of a nucleic acid encoding progranulin, optionally human progranulin.

E2. The population of modified HSPCs of E1, wherein the cells comprise on average between 1 and 10, such as 2-5 or 4-8 copies of progranulin.

E3. The population of modified HSPCs of E1 or E2, wherein the cells express progranulin.

E4. The population of modified HSPCs of any one of E1 to E3, wherein the nucleic acid is a genomic DNA or a complementary DNA (cDNA).

E5. The population of modified HSPCs of any one of E1 to E4, wherein the nucleic acid encoding progranulin comprises the sequence of SEQ ID NO: 1, or a sequence with 95% sequence identity thereto.

E6. The population of modified HSPCs of any one of E1 to E5, wherein the progranulin encoded by the exogenous copy of a nucleic acid comprises the sequence of SEQ ID NO: 2, or a sequence with 95% sequence identity thereto.

E7. The population of modified HSPCs of any one of E1 to E6, wherein the nucleic acid is integrated into the genome of the cells.

E8. The population of modified HSPCs of any one of E1 to E7, wherein the nucleic acid is introduced into the cells via a viral vector.

E9. The population of modified HSPCs of E8, wherein the viral vector is a lentiviral vector.

E10. The population of modified HSPCs of E9, wherein the lentiviral vector comprises the GRN cDNA under the control of the human phosphoglycerate kinase (hPGK) promoter.

E11. The population of modified HSPCs of E9, wherein the lentiviral vector comprises the GRN cDNA under the control of a human HLA-DRA derived promoter, such as the promoter comprising SEQ ID NO:8, or a sequence with at least 90% sequence identity thereto.

E12. The population of modified HSPCs of any one of the preceding embodiments, wherein the HSPC also comprises an exogenously introduced nucleic acid that encodes a metallothionein, such as Metallothionein 1G.

E13. The population of modified HSPCs of any one of the preceding embodiments, wherein the HSPC cells are mammalian cells, suitably human cells.

E14. The population of modified HSPCs of any one of the preceding embodiments, wherein prior to the modification, the HSPCs are obtained from the bone marrow, umbilical cord, amniotic fluid, chorionic villi, cord blood, placental blood or peripheral blood.

E15. The population of modified HSPCs of E14, wherein the HSPCs are obtained from mobilized peripheral blood or bone marrow.

E16. The population of modified HSPCs of any one of the preceding embodiments, wherein the HSPCs are derived from a healthy individual.

E17. The population of modified HSPCs any one of the preceding embodiments, wherein the HSPCs are derived from an individual with a diagnosed disease or disorder.

E18. The population of modified HSPCs of any one of the preceding embodiments, wherein the HSPC cells are ex vivo cultured before or after or both before and after the introduction of the exogenous copy of a nucleic acid encoding progranulin.

E19. The population of modified HSPCs of any one of the preceding embodiments, wherein the HSPC cells are produced by a method comprising:

a) contacting a sample of unmodified HSPCs with a nucleic acid carrying an exogenous copy of a nucleic acid encoding progranulin; and

b) allowing the nucleic acid encoding progranulin to integrate into the genome of the cells so as to produce a population of modified HSPCs. E20. The population of modified HSPCs of E19, wherein the method further comprises establishing that there is at least one-fold increase in the number of progranulin expressing cells compared to non-modified cells.

E21. An ex vivo method for producing a population of hematopoietic stem cells (HSPCs) modified to express progranulin, the method comprising:

a) contacting a sample of HSPCs with a vector carrying an exogenous copy of a nucleic acid encoding progranulin, and optionally an exogenous copy of a nucleic acid encoding metallothionein, under conditions to allow the vector to transduce the HSPCs; and

b) culturing the cells in step (a) to produce a population of modified HSPCs cells expressing progranulin, and optionally also metallothionein.

E22. The method of E21, wherein before, during or after contact with the viral vector the HSPC are cultured in a medium comprising one or more transduction enhancers.

E23. The method of E22, wherein the method further comprises establishing that there is at least one-fold increase in the number of progranulin expressing cells compared to non-modified cells.

E24. The method of E21, E22 or E23, wherein the sample of HSPC is obtained from bone marrow, umbilical cord, amniotic fluid, chorionic villi, cord blood, placental blood or peripheral blood.

E25. The method of E24, wherein the sample of HSPC is obtained from mobilized peripheral blood or bone marrow.

E26. The method of any one of E21 to E25, wherein the sample of HSPCs is obtained from a healthy individual.

E27. The method of E26, wherein the sample of HSPCs is obtained from an individual with FTD.

E28. The method of any one of the E1 to E27, wherein the HSPCs in the sample are CD34⁺ and/or CD45⁺.

E29. The method of any one of claims E21 to E28, wherein the vector is viral vector.

E30. The method of E29, wherein the viral vector is a lentiviral vector.

E31. The method of any one of E21 to E28, wherein the nucleic acid is integrated into the genome of the cells.

E32. A population of progranulin expressing HSPCs produced by the method of any one of claims E21 to E31.

E33. A population of HSPCs expressing exogenously introduced progranulin and metallothionein produced by the method of any one of claims E21 to E31.

E34. A composition comprising the HSPCs of any one of E1 to E20, E32 or E33.

E35. A composition for transplantation into a subject, the composition comprising the HSPCs of any one of E1 to E20, 32 or E33.

E36. A method of treating frontotemporal dementia (FTD) in a subject in need thereof, the method comprising administering to the subject the composition of E35.

E37. The method of E36, wherein the introduction of the HSPCs results in an increase in granulin levels in brain tissue, in particular in microglia/microglia-like, transplant derived cells.

E 38. The method of E36 or E37, wherein the HSPCs are autologous to the recipient subject.

E39. The method of E36 or E37, wherein the HSPCs are non-autologous and allogenic to the recipient subject.

E40. The method of E36 or E37, wherein the HSPCs are non-autologous and xenogeneic to the recipient subject.

E 41. The method of any one of E36 to E40, wherein the HSPCs are administered to the patient via intravenous (IV) and/or intracerebroventricular (ICV) and/or intrathecal lumbar (ITL) injection.

E42. A composition comprising the HSPCs of any one of E1 to E20, E32 or E33 for use in the prevention or treatment of a neurodegenerative disease or disorder selected from FTD and NCL.

E43. A population of progranulin expressing HPSCs of any one of E1 to E20, E32 or E33 for use in the prevention or treatment of a neurodegenerative disease or disorder selected from FTD and NCL.

E44. A population of modified hematopoietic stem cells (HSPCs) where the cells carry at least one, such as between 2 and 8, exogenous copy of a nucleic acid encoding human progranulin under the control of a promoter that is inducible by an inflammatory cytokine, such as interferon gamma, optionally wherein the promoter is derived from HLA-DRA, such as one comprising the sequence disclosed in SEQ ID NO 8, or a sequence having at least 90% sequence identity thereto.

E45. A population of modified hematopoietic stem cells (HSPCs) where the cells carry at least one exogenous copy of a nucleic acid encoding human progranulin under the control of a synthetic promoter derived from HLA-DRA, such as one comprising the sequence disclosed in SEQ ID NO 8, or a sequence having at least 90% sequence identity thereto.

The following examples provide those of ordinary skill in the art with a complete description of how to make and use the compositions and therapeutic methods of the disclosure and are not intended to limit the scope of what the inventors regard as their invention.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Transduction of GRN^{-/-} HAP1 and Hela Cell Lines with LVs Encoding the Human GRN

(i) Vector Construction, Production and Characterization

The mouse GRN (mGRN), human GRN wild-type (hGRN_wt) (as in SEQ ID NO: 1) or human GRN codon optimized (hGRN_co) (75% homology with wild-type sequence) cDNAs were sub-cloned into the pCCL.PGK.WPREmut-kana back bone lentivirus (LV) transfer plasmid by Genewiz® (Leipzig, Germany). pCCL.PGK.WPREmut-kana is a HIV-based, LV lentivirus expression plasmid in which the transcript is driven by the ubiquitous human phosphoglycerate kinase (hPGK) promoter. The plasmid contains all the viral processing elements for the production of self-inactivating (SIN) 3^{rd} generation lentivirus particles together with a pUC origin of amplification and an E. coli Kanamicin resistance. The cDNAs were designed to contain the complete GRN (mouse or human) ORF together with the kozak consensus sequence, and to be flanked by the restriction enzyme sites BamHI and Sall1, in order to sub-clone the transgene into the LV backbone with the correct orientation. Upon receipt from Genewiz, the three LV plasmids were transformed into DH5α competent E. coli cells, single colonies were grown, LV plasmid DNA was extracted, and the presence and size of the cDNA inserts checked by diagnostic enzymatic digestion.

LV particles were produced by transient transfection of HEK293T cells with 4 different packaging plasmids (pEnv, pGag-Pol, pRev, pAdvantage), together with the transgene, by calcium phosphate method following standard operating procedures. The LVs were then concentrated by ultracentrifugation, re-suspended in phosphate buffered saline (PBS) and stored at −80° C. until use. Each LV (mGRN_LV, hGRN_wt_LV and hGRN_co_LV) was then functionally titered in order to measure infectious viral particles. LV titer (Transduction Unit/ml) was calculated by transducing HEK293T cells with serial dilutions of the concentrated LV and estimating vector copy number (VCN) integration events through droplet digital PCR method (ddPCR). Notably, the titer for all the LVs produced was in the range of 10⁹ TU/ml. Finally, to confirm functionality of the vectors, GRN (human or murine) expression was evaluated on transduced cells by western blot (WB) analysis.

Key Sequences:

Mouse GRN (mGRN) Sequence Used:

(SEQ ID NO: 5)
GGATCCGCCACCATGTGGGTCCTGATGAGCTGGCT
GGCCTTCGCGGCAGGGCTGGTAGCCGGAACACAGT
GTCCAGATGGGCAGTTCTGCCCTGTTGCCTGCTGC
CTTGACCAGGGAGGAGCCAACTACAGCTGCTGTAA
CCCTCTTCTGGACACATGGCCTAGAATAACGAGCC
ATCATCTAGATGGCTCCTGCCAGACCCATGGCCAC
TGTCCTGCTGGCTATTCTTGTCTTCTCACTGTGTC
TGGGACTTCCAGCTGCTGCCCGTTCTCTAAGGGTG
TGTCTTGTGGTGATGGCTACCACTGCTGCCCCCAG
GGCTTCCACTGTAGTGCAGATGGGAAATCCTGCTT
CCAGATGTCAGATAACCCCTTGGGTGCTGTCCAGT
GTCCTGGGAGCCAGTTTGAATGTCCTGACTCTGCC
ACCTGCTGCATTATGGTTGATGGTTCGTGGGGATG
TTGTCCCATGCCCCAGGCCTCTTGCTGTGAAGACA
GAGTGCATTGCTGTCCCCATGGGGCCTCCTGTGAC
CTGGTTCACACACGATGCGTTTCACCCACGGGCAC
CCACACCCTACTAAAGAAGTTCCCTGCACAAAAGA
CCAACAGGGCAGTGTCTTTGCCTTTTTCTGTCGTG
TGCCCTGATGCTAAGACCCAGTGTCCCGATGATTC
TACCTGCTGTGAGCTACCCACTGGGAAGTATGGCT
GCTGTCCAATGCCCAATGCCATCTGCTGTTCCGAC
CACCTGCACTGCTGCCCCCAGGACACTGTATGTGA
CCTGATCCAGAGTAAGTGCCTATCCAAGAACTACA
CCACGGATCTCCTGACCAAGCTGCCTGGATACCCA
GTGAAGGAGGTGAAGTGCGACATGGAGGTGAGCTG
CCCTGAAGGATATACCTGCTGCCGCCTCAACACTG
GGGCCTGGGGCTGCTGTCCATTTGCCAAGGCCGTG
TGTTGTGAGGATCACATTCATTGCTGCCCGGCAGG
GTTTCAGTGTCACACAGAGAAAGGAACCTGCGAAA
TGGGTATCCTCCAAGTACCCTGGATGAAGAAGGTC
ATAGCCCCCCTCCGCCTGCCAGACCCACAGATCTT
GAAGAGTGATACACCTTGTGATGACTTCACTAGGT
GTCCTACAAACAATACCTGCTGCAAACTCAATTCT
GGGGACTGGGGCTGCTGTCCCATCCCAGAGGCTGT
CTGCTGCTCAGACAACCAGCATTGCTGCCCTCAGG
GCTTCACATGTCTGGCTCAGGGGTACTGTCAGAAG
GGAGACACAATGGTGGCTGGCCTGGAGAAGATACC
TGCCCGCCAGACAACCCCGCTCCAAATTGGAGATA
TCGGTTGTGACCAGCATACCAGCTGCCCAGTAGGG
CAAACCTGCTGCCCAAGCCTCAAGGGAAGTTGGGC
CTGCTGCCAGCTGCCCCATGCTGTGTGCTGTGAGG
ACCGGCAGCACTGTTGCCCGGCCGGGTACACCTGC
AATGTGAAGGCGAGGACCTGTGAGAAGGATGTCGA
TTTTATCCAGCCTCCCGTGCTCCTGACCCTCGGCC
CTAAGGTTGGGAATGTGGAGTGTGGAGAAGGGCAT
TTCTGCCATGATAACCAGACCTGTTGTAAAGACAG
TGCAGGAGTCTGGGCCTGCTGTCCCTACCTAAAGG
GTGTCTGCTGTAGAGATGGACGTCACTGTTGCCCC
GGTGGCTTCCACTGTTCAGCCAGGGGAACCAAGTG
TTTGCGAAAGAAGATTCCTCGCTGGGACATGTmTG
AGAGATCCGGTCCCAAGACCGCTACTGTAAGTCGA
C

Human GRN codon optimized sequence (hGRN.co) used:

(SEQ ID NO: 6)
GGATCCGCCACCATGTGGACACTGGTCAGCTGGGTG
GCCCTCACAGCTGGACTGGTGGCTGGCACAAGATG
TCCCGACGGCCAGTTTTGCCCCGTGGCTTGTTGCC
TCGACCCCGGAGGCGCTTCCTACAGCTGCTGTAGA
CCTCTGCTGGATAAATGGCCTACAACACTGAGCAG
ACATCTGGGCGGCCCTTGCCAAGTCGATGCTCATT
GCTCCGCCGGCCACAGCTGCATCTTCACAGTGTCC
GGAACATCCAGCTGCTGCCCCTTTCCCGAAGCTGT
CGCTTGCGGAGACGGCCATCACTGCTGTCCTAGAG
GCTTTCACTGCAGCGCTGACGGCAGAAGCTGCTTC
CAGAGGAGCGGAAACAACAGCGTGGGAGCCATTCA
ATGTCCCGACAGCCAGTTCGAATGCCCCGACTTTA
GCACATGCTGCGTGATGGTGGACGGCAGCTGGGGA
TGTTGCCCCATGCCCCAAGCCAGCTGCTGCGAGGA
TAGAGTCCACTGTTGTCCCCACGGAGCTTTTTGCG
ATCTGGTGCATACAAGATGCATCACCCCCACCGGC
ACCCACCCTCTGGCCAAGAAACTGCCCGCTCAGAG
GACAAATAGAGCTGTGGCTCTGAGCAGCAGCGTCA
TGTGTCCCGACGCTAGGTCTAGATGCCCCGACGGC
TCCACATGCTGTGAACTGCCCTCCGGCAAGTATGG
CTGCTGCCCTATGCCCAATGCTACATGCTGCTCCG
ATCATCTGCATTGCTGCCCCCAAGACACCGTGTGT
GATCTGATTCAGAGCAAGTGCCTCTCCAAGGAGAA
CGCCACCACAGATCTGCTGACCAAGCTCCCCGCCC
ACACAGTGGGCGATGTGAAGTGTGACATGGAGGTG
AGCTGCCCCGACGGATACACATGCTGTAGGCTGCA
GTCCGGCGCTTGGGGATGCTGTCCCTTCACCCAAG
CCGTGTGCTGTGAAGACCATATCCATTGTTGCCCC
GCTGGATTCACATGCGACACCCAAAAGGGCACATG
CGAGCAAGGCCCTCATCAAGTGCCTTGGATGGAGA
AAGCCCCCGCTCATCTGTCTCTGCCCGATCCCCAA
GCTCTGAAGAGGGACGTGCCTTGCGATAACGTGAG
CAGCTGCCCTTCCAGCGACACATGCTGTCAACTGA
CCAGCGGAGAGTGGGGCTGTTGCCCTATCCCCGAG
GCTGTCTGCTGTAGCGACCACCAGCACTGTTGTCC
TCAAGGATACACATGCGTGGCCGAAGGACAATGTC
AGAGGGGCTCCGAGATCGTGGCTGGACTCGAGAAG
ATGCCCGCCAGAAGAGCCTCTCTGAGCCATCCTAG
AGACATCGGATGCGACCAGCATACAAGCTGCCCCG
TGGGCCAGACATGTTGCCCCTCTCTGGGCGGAAGC
TGGGCTTGTTGCCAACTCCCCCACGCCGTCTGTTG
TGAGGATAGACAACATTGCTGTCCCGCCGGCTACA
CATGCAACGTCAAGGCCAGAAGCTGCGAAAAGGAG
GTGGTGTCCGCCCAGCCCGCCACATTTCTGGCTAG
ATCCCCCCATGTGGGCGTGAAGGATGTGGAGTGCG
GAGAGGGACACTTTTGCCACGACAACCAGACATGC
TGCAGAGACAATAGGCAAGGCTGGGCTTGCTGCCC
TTATAGGCAAGGCGTGTGCTGTGCTGATAGAAGGC
ACTGTTGCCCCGCCGGCTTCAGATGCGCCGCCAGA
GGCACCAAATGTCTGAGAAGAGAGGCTCCTAGGTG
GGACGCTCCTCTGAGAGATCCCGCTCTGAGGCAGC
TGCTGTGAGTCGAC

Human PGK promoter sequence used:

(SEQ ID NO: 7)
ATCATCGATTTCCGAATTCCACGGGGTTGGGGTTG
CGCCTTTTCCAAGGCAGCCCTGGGTTTGCGCAGGG
ACGCGGCTGCTCTGGGCGTGGTTCCGGGAAACGCA
GCGGCGCCGACCCTGGGTCTCGCACATTCTTCACG
TCCGTTCGCAGCGTCACCCGGATCTTCGCCGCTAC
CCTTGTGGGCCCCCCGGCGACGCTTCCTGCTCCGC
CCCTAAGTCGGGAAGGTTCCTTGCGGTTCGCGGCG
TGCCGGACGTGACAAACGGAAGCCGCACGTCTCAC
TAGTACCCTCGCAGACGGACAGCGCCAGGGAGCAA
TGGCAGCGCGCCGACCGCGATGGGCTGTGGCCAAT
AGCGGCTGCTCAGCAGGGCGCGCCGAGAGCAGCGG
CCGGGAAGGGGCGGTGCGGGAGGCGGGGTGTGGGG
CGGTAGTGTGGGCCCTGTTCCTGCCCGCGCGGTGT
TCCGCATTCTGCAAGCCTCCGGAGCGCACGTCGGC
AGTCGGCTCCCTCGTTGACCGAATCACCGACCTCT
CTCCCCAACGCG.

(ii) Transduction of GRN^{-/-} human cell lines (MOI test)

HAP1 (GRN^{-/-} and wt) and Hela (GRN^{-/-} and wt) cell lines were obtained through the Bluefield Project to Cure FTD. GRN^{-/-} HAP1 cells are a near-haploid cell line possessing GRN ablation. Hap1 cells were cultured as adherent cells in Iscove's Modified Dulbecco's Medium (IMDM) containing 10% of fetal bovine serum (FBS), 1% Glutamine (Q) and 1% Penicillin/Streptomycin (Pen/Streo). GRN^{-/-} Hela cells were generated through CRISPR gRNA-Cas9 technology and were cultured as adherent cells in Dulbecco's Modified Eagle's Medium (DMEM) containing 10% FBS, 1% G and 1% Pen/Strep. To estimate the optimal titre that would lead to high transduction efficiency and low toxicity in GRN^{-/-} HAP1 and GRN^{-/-} Hela cell lines, a dose response experiment was performed. To optimize LV transduction, cells were transduced at different multiplicity of infection (MOI) (1, 3 and 10) with mGRN, hGRN_wt or hGRN_co LV. In parallel, cells were also transduced with LV encoding for the green fluorescent protein (GFP) in order to evaluate transduction efficiency by flow cytometry analysis. GRN^{-/-} HAP1 and Hela cells were seeded at low density (1×10⁵—Hela; 2×10⁵ HAP1) in 12-well plastic culture plates and incubated under standard culture conditions for 6 hours, to allow the cells to properly attach to the culture plate. Cells were then transduced for 16 hours with a final volume of 0.5 ml/well of complete culture medium containing LV. Control untransduced cells were cultured under the same conditions without the LV. The following day, the lentivirus containing medium was discarded, cells were washed with PBS and 1 ml of fresh culture medium added. Cells were left in an incubator at 37° C., 5% CO₂. Cells were passaged for at least 12 days following LV infection before being processed for further analysis.

(iii) Characterization of GRH-Corrected GRN^{-/-} Human Cell Lines by WB and FC

To evaluate transduction efficiency, GRN^{-/-} HAP1 and Hela cells transduced with the GFP_LV at different MOIs, were collected and quantification of GFP-positive cells evaluated by FC analysis. Untransduced cells were used as control. With the transduction protocol described above, for both cell lines, the transduction efficiency improved with increasing MOIs. In particular, with a MOI of 1, 50% of cells were successfully transduced, while approximately 80% and 97% of cells were transduced with a MOI of 5 and 10 respectively.

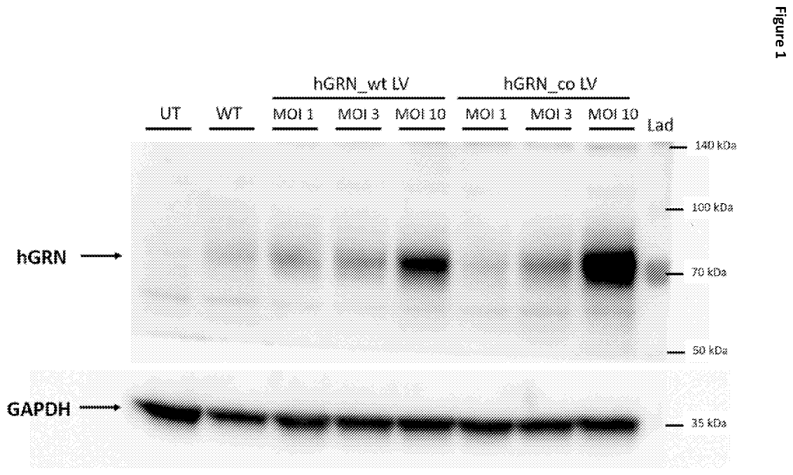

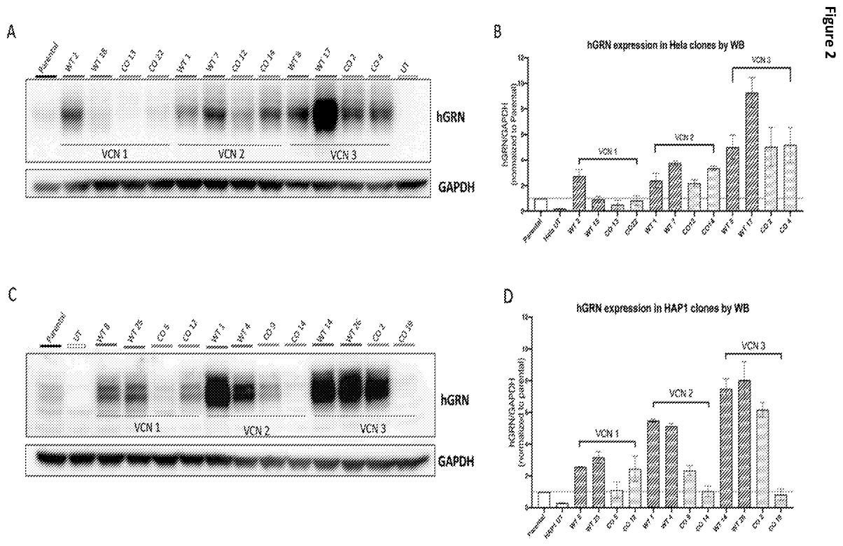

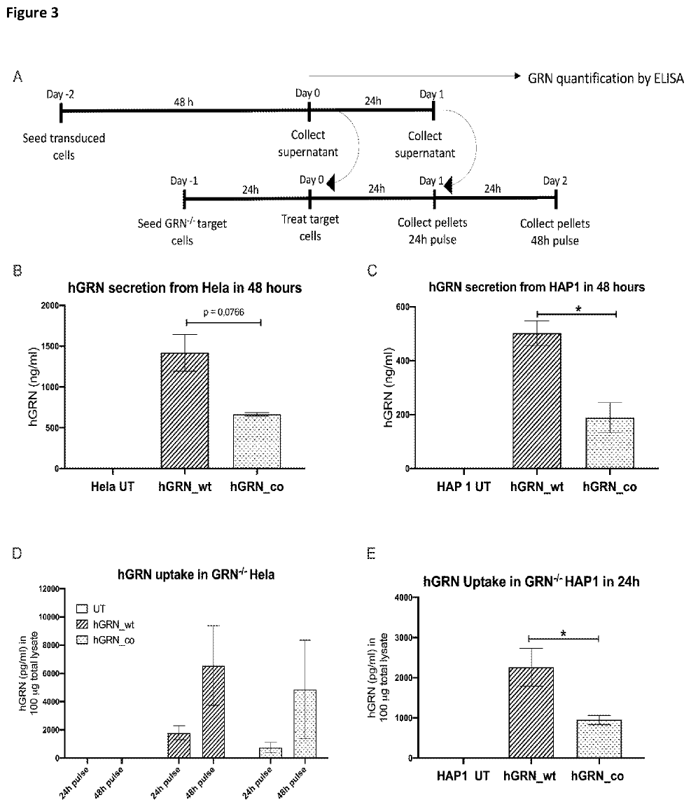

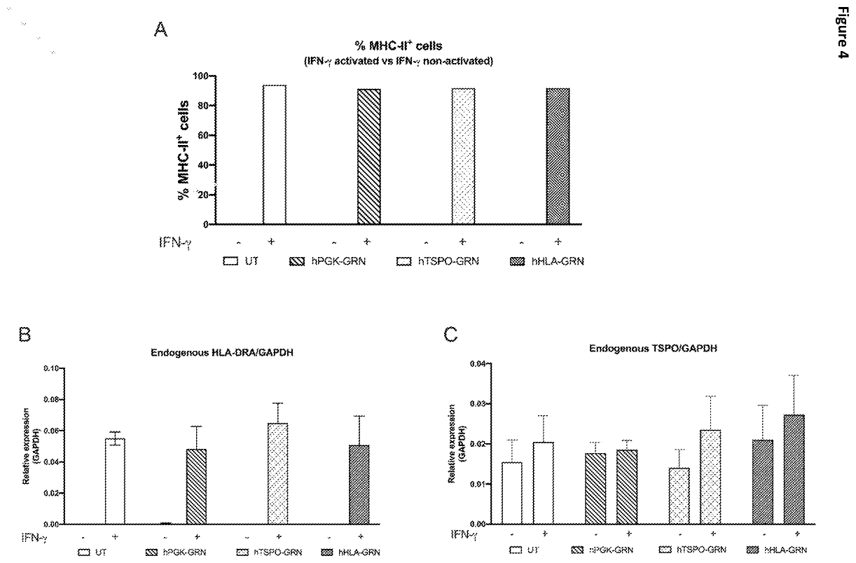

GRN^{-/-} HAP1 and Hela cells transduced with the three LVs encoding for the murine or human GRN were processed to evaluate and compare GRN expression by WB analysis. FIG. 1 shows a representative immunoblot in which the expression of human GRN is compared between GRN^{-/-} HAP1 untransduced cells (negative control), wild-type HAP1 cells (parental control) and GRN^{-/-} HAP1 cells transduced with either the hGRN_wt_LV or hGRN_co_LV at different MOIs. As can be seen in the figure, in cells infected with the two LVs at a MOI of 1, the levels of GRN expression are comparable to those seen in control wild-type HAP1 cells, while no expression of GRN is detected in untransduced GRN^{-/-} HAP1 cells. Notably, extensive GRN overexpression is observed in cells infected with LVs at MOI of 3 and 10. Moreover, it is evident that the LV encoding for the codon optimized version of GRN can induce greater protein expression.

### Example 2. Genetic Correction and Cross-Correction of GRN^{-/-} Cell Lines with LVs Encoding the Human GRN

(i) Transduction of GRN^{-/-} Hela and HAP1 Cell Lines and Generation of Single Cell Clones.

HAP1 and Hela GRN^{-/-} cells were transduced with the hGRN wt or hGRN_co LVs at a MOI of 3. Cells were seeded at low density (1×10⁵—Hela; 2×10⁵ HAP1) in 12-well plastic culture plates and incubated under standard culture conditions for 6 hours, to allow the cells to properly attach to the culture plate. Cells were then transduced for 16 hours with a final volume of 0.5 ml/well of complete culture medium containing LV. Control untransduced cells were cultured under the same conditions without the LV. The following day, the lentivirus containing medium was discarded, cells were washed with PBS and 1 ml of fresh culture medium added. Cells were left in an incubator at 37° C., 5% CO₂. Cells were passaged for at least 14 days following LV infection before being processed for VCN determination by digital droplet PCR (ddPCR). Upon confirmation of successful LVs transduction, single cell clones were produced to evaluate and compare hGRN expression driven by the hGRN_wt and hGRN_co LV at a specific VCN. Single cell clones from transduced HAP1 and Hela cells were generated through single cell sorting with the BD FACSAria™ flow cytometer. For each cell line, ˜20 single cell clones were cultured and expanded. Transduced clonal populations were finally screened for VCN by ddPCR to select clones with a VCN of 1, 2 or 3. At the end of the screening process we were able to obtain at least 2 different cell clones for each VCN (1,2 and 3) for both the hGRN_wt and hGRN_co LV groups.

(ii) Comparison of GRN Expression in GRN^{-/-} Cells Genetically Corrected with the hGRN_Wt_LV Vs the hGRN_Co_LV by Western Blot.

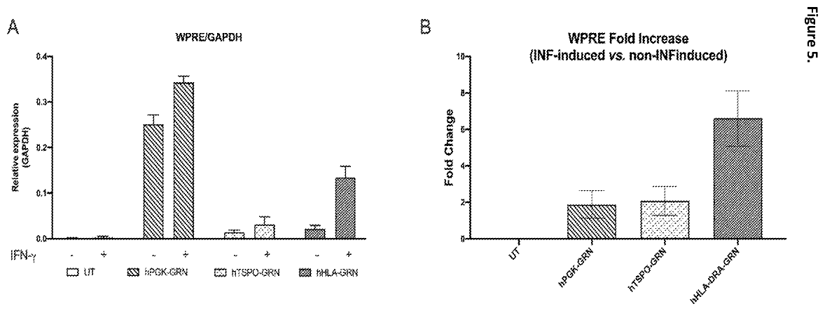

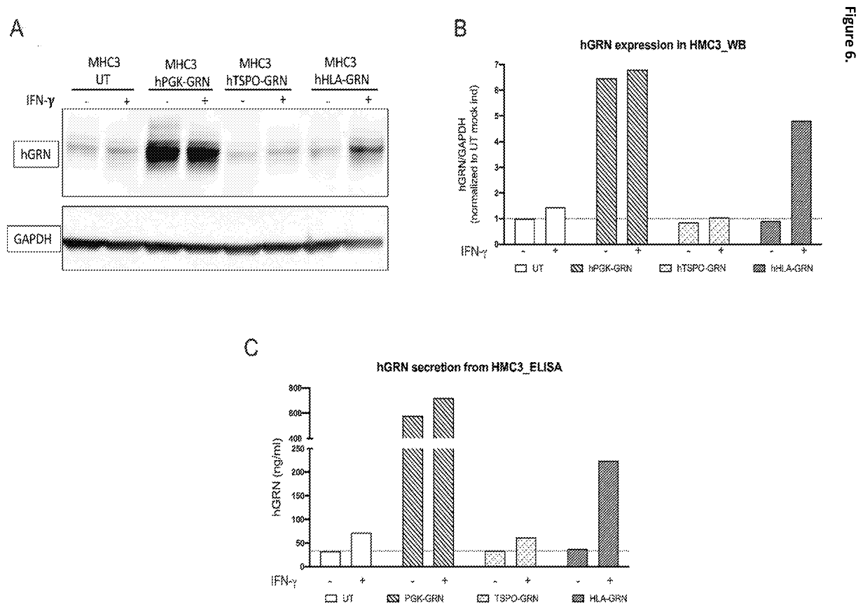

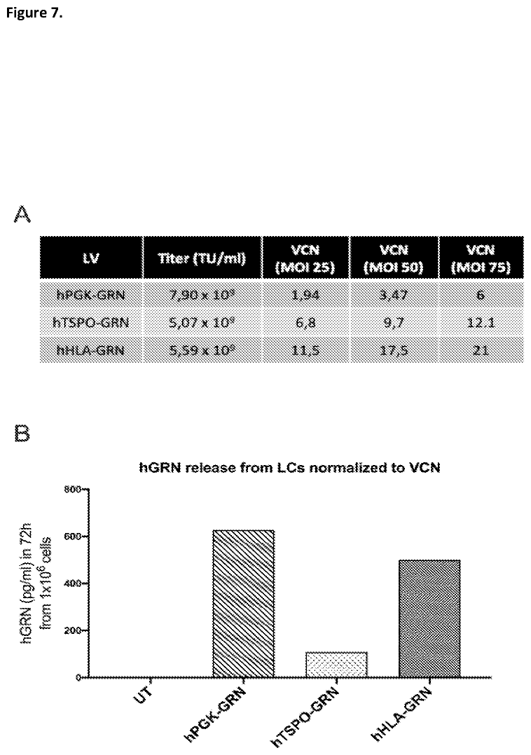

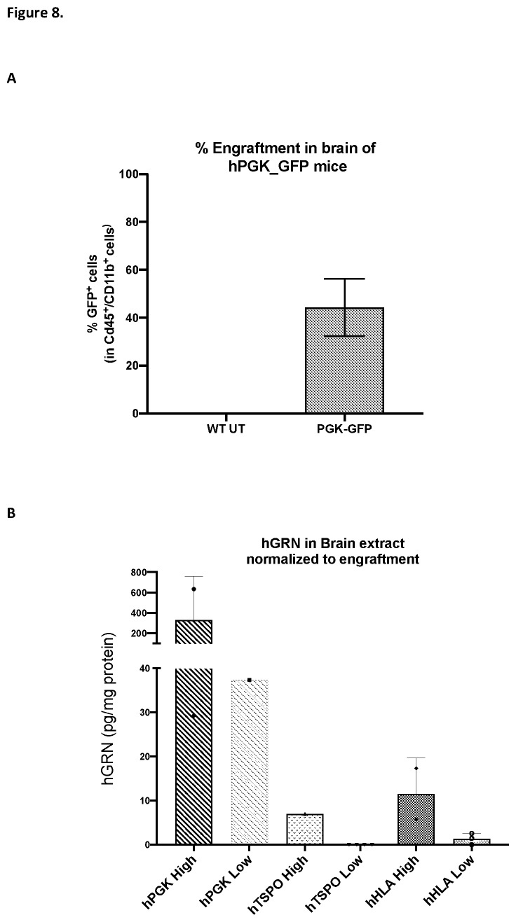

GRN^{-/-} HAP1 and Hela transduced cell clones were processed to evaluate and compare GRN expression by WB analysis on total cell lysates using an anti-human GRN specific antibody. FIG. 2A shows a representative immunoblot in which the expression of human GRN is compared between GRN^{-/-} untransduced (UT—negative control) cells, wild-type Hela (parental) cells and GRN^{-/-} genetically corrected Hela cell clones, transduced with either the hGRN_wt_LV or hGRN_co_LV, and bearing 1,2 or 3 LV copies (VCN). The immunoblot shows the hGRN specific band in parental (positive control) and transduced cells, while no band is detected in control UT GRN^{-/-} cells. The band was of the expected size, at a level between the 70 and the 85 kDa bands of the protein ladder. GAPDH (37 kDa) was used as internal loading control. As shown by immunoblot densitometry analysis (FIG. 2B), there was a clear correlation between the hGRN expression levels and VCN in both hGRN_wt and hGRN_co clones, confirming that higher levels of protein expression can be achieved by increasing the number of LV integration copies in the genome of the transduced cells. Notably, protein expression was generally higher in Hela hGRN_wt clones when compared to hGRN_co clones, at every VCN range analyzed. Higher hGRN expression in hGRN_wt clones, compared to hGRN_co, was also observed in genetically corrected GRN^{-/-} HAP1 cells (FIG. 2C), as confirmed by immunoblot densitometry analysis (FIG. 2D).

(iii) Comparison of GRN Release in Expression in GRN^{-/-} Cells Genetically Corrected with the hGRN_Wt_LV Vs the hGRN_Co_LV and Evaluation of Cross-Correction Ability by ELISA.

To confirm that hGRN could be correctly processed and released from genetically corrected GRN^{-/-} cells and subsequently internalized by GRN^{-/-} cells, we performed in vitro cross-correction experiments employing GRN^{-/-} Hela or HAP1 hGRN_wt or hGRN_co transduced clones as producing cells and GRN^{-/-} Hela or HAP1 untransduced cells as target cells (FIG. 3A). First, hGRN_wt or hGRN_co LV transduced Hela and HAP1 cell clones (VCN 3) were cultured at cell confluence for 48 hours and supernatants collected to measure and compare hGRN release in the medium by ELISA assay, using a commercially available hGRN ELISA KIT (Abcam, ab252364) and following the manufacturer's instructions. As shown in FIG. 3, detectable levels of hGRN, expressed as nanograms per milliliter (ng/ml), could be detected in the culture media of both Hela (FIG. 3B) and Hap1 (FIG. 3C) transduced cell clones, while no signal was detected in control untransduced (UT) GRN^{-/-} cell media. Importantly, cells transduced with the hGRN_wt LV secreted significantly higher levels of hGRN protein in comparison to cells transduced with the hGRN_co LV. This data was in line with the western blot data, confirming the superiority of the wild type cDNA (hGRN_wt), over the codon-optimized cDNA (hGRN_co), in inducing protein overexpression upon genetic correction of GRN^{-/-} cell lines. The capacity of LV-transduced cells (producing cells) to cross-correct GRN^{-/-} target cells was then evaluated. Briefly, Hela or HAP1 LV-transduced cells were cultured at cellular confluence for 48-72 hours and cell culture supernatant collected and used to treat GRN^{-/-} target cells. At the end of the treatment (24-48 hours) target cells were collected and total protein extracted to measure the levels of internalized hGRN by ELISA assay. As a mock treatment condition, GRN^{-/-} target cells were treated with medium collected from control untransduced cells. As shown in FIG. 3D, hGRN was detected in GRN^{-/-} Hela target cells following 24-hour treatment with supernatant from hGRN_wt and hGRN_co LV-transduced Hela cells, indicating a successful cross-correction. Interestingly, a longer duration treatment (48 hours), allowed for hGRN build-up in target cells. As expected, hGRN was not detectable in cells treated with medium from GRN^{-/-} UT cells. Importantly, hGRN was detected in GRN^{-/-} HAP1 cells following treatment with supernatant obtained from hGRN_wt and hGRN_co LV-transduced cells, with the amount of internalized hGRN that was significantly higher in cells treated with the hGRN_wt medium compared to hGRN_co. hGRN was not detectable in cells treated with medium from GRN^{-/-} UT cells. Overall, these results demonstrate that upon lentiviral transduction with LVs encoding for the hGRN, GRN^{-/-} cells can be efficiently corrected and induced to express and produce biologically relevant levels of hGRN, which upon release in the culture media maintain protein stability as shown by protein internalization in target cross-corrected GRN^{-/-} cells. Moreover, the hGRN wild type cDNA sequence (hGRN_wt) demonstrated to be superior to the codon-optimized cDNA (hGRN_co), and thus selected as the best transgene for further investigations.

### Example 3. Evaluation and Comparison of LV-Derived hGRN cDNA Expression Driven by the Synthetic hTSPO and hHLA Inducible Promoters In Vitro in Comparison to the Constitutive hPGK Promoter

(i) Production of Lentiviral Vectors Carrying the Human Translocator Protein Promoter (hTSPO) or hHLA Promoter to Drive the Human GRN.

Two different HIV-based lentivirus (LV) expression vectors were used to drive the expression of GRN in a myeloid-specific and inducible manner: i) the pCCL.HLA-DRA.WPREmut-kana LV vector in which the transcript is driven by an innovative microglia/myeloid regulated promoter based on the minimally regulatory elements of the human HLA-DRA promoter (SEQ ID NO: 8); and ii) the pCCL.sinPPT.hTspo(MPP01)wPRE vector in which the transgene is driven by an innovative, ad hoc-designed, inducible promoter derived from the human TSPO (MPP01(hTSPO_prximal5′prom)(“P1”) in International Patent Publication No. WO2021067629; SEQ ID NO: 9 herein). The pCCL.PGK.WPREmut-kana LV vector, in which the transcript is driven by the ubiquitous human phosphoglycerate kinase (hPGK) promoter, was used as control. The human GRN_wild type (hGRN_wt) cDNA was sub-cloned into the 2 novel lentiviral transfer plasmids by Genewiz® (Leipzig, Germany). The original vectors contain all the viral processing elements for the production of self-inactivating (SIN) 3^{rd} generation lentivirus particles together with a pUC origin of amplification and an E. coli Kanamicin or Ampicillin resistance. Upon receipt from Genewiz, the three LV plasmids were transformed into DH5α competent E coli cells, single colonies grown, LV plasmid DNA extracted, and the presence and size of the cDNA inserts checked by diagnostic enzymatic digestion. LV particles were produced by transient transfection of HEK293T cells with the packaging plasmids (pEnv, pGag-Pol, pRev, pAdvantage), together with the transfer plasmid, by calcium phosphate method following standard operating procedures. The LVs were then concentrated by ultracentrifugation, re-suspended in phosphate buffered saline (PBS) and stored at −80° C. until use. Each LV (hPGK-GRN, hTSPO-GRN and hHLA-DRA-GRN) was then functionally titred to measure infectious viral particles. LV infectious titers were determined as Transducing Units per ml (TU/ml) of viral stock by evaluating the resulting Vector Copy Number (VCN) in HEK293T cells transduced with serial dilutions of each LV stock, after two weeks in culture. VCN was determined by digital droplet PCR (ddPCR). In several productions, all the LVs could be produced with an Infectious Titer generally above 10⁹ TU/ml.

SEQ ID NO: 9 (MPP01(hTSPO_prximal5′prom)(“P1”):
Tgcatcaccgcgttgcggcctcatcagtcccacga
ctttgtgcccattttactcatgaggagatggaggc
ccagagagccagtcagaaagtggctgggccaggac
taagagtgcagcgcgctgcctccgtgccctgcgtc
aacagctcaaggaactggggtgtccggaaatgggg
ccaaggctgctgggcagcaggacgctcagggcctt
ggcctcaggagagcaaattccccactcggagatcg
gtcttgttgctgcattttattcatgggaaatctga
ggctagaagagacgacaaacgacacgccgttggac
acacggcaacgttttagatgttgggtctggccggg
cggccgtcaccggtcaccatggggaggaggaggag
ccgagagacttgctcgcggccggggggaggcagaa
gcgcgtcccgcgggagaggtggctttgaggagtga
gctcccggtcccgcggggacgcgagtgggcccagt
gcccgggctgccaggcggggcggggcggggccggg
cgactgagaggggcggggcctggcggctgggaggg
gCGGGGCGGATGCGGGGACAGCGGCCTGGCTAACT
CCTGCACGGCAGTGCCCTTCCCGGAGCGTGCCCTC
GCCG

The P1 promoter (disclosed in WO2021067629) comprises 635 bp that correspond to nucleotide residues −562 to +73 (capital letters) of the hTspo immediate 5′ promoter.

(ii) Transduction of HMC3 Human Cell Line with GRN LVs.

HMC3 cells (ATCC® CRL-3304™) were obtained from ATCC®. HMC3 cells are a human microglia transformed cell line retaining the properties of primary microglia cells. Resting HMC3 cells strongly express the microglia/macrophage marker IBA1, and upon activation by IFN-gamma, they upregulate markers of activated microglia such as MHC-II, CD68 and CD11b. HMC3 cells are cultured as adherent cells in Iscove's Modified Dulbecco's Medium (IMDM) containing 10% of fetal bovine serum (FBS), 1% Glutamine (Q) and 1% Penicillin/Streptomycin (Pen/Strep). HMC3 cells were mock-transduced or transduced with the hPGK-GRN, hTSPO-GRN or the hHLA-GRN LVs for 16 hours at a MOI ranging from 1 to 30. After two weeks in culture, mock- and LV-transduced cells were collected, and the genomic DNA was extracted for VCN determination by ddPCR. HMC3 resulted efficiently transduced at all the MOIs tested, with VCNs correlating with the increase of MOIs (data not shown). Bulk cell populations of mock-transduced and LV-transduced cells with an average VCN of 2 were selected for further experiments.

(iii) Evaluation of the GRN Gene Expression in LV-Transduced HMC3 Cells Before and After Cell-Activation

To test the capability of the hTSPO and hHLA promoter to drive GRN expression in a robust and regulated fashion, bulk cell populations of mock-transduced and LV-transduced cells with an average VCN of 2 were activated by incubation with IFNγ 100 ng/ml for 72 hours to evaluate the hTSPO and hHLA-driven transgene expression in resting vs. activated cell conditions. Mock-activated cells were maintained in the same culture conditions without IFNγ. Briefly, 1×10⁵ cells were seeded in 6-well plastic culture plates and incubated with 100 ng/ml IFNγ for a total of 72 hours. For optimal induction, fresh media containing IFNγ was added every 24 hours. As a positive cell marker of microglia activation, the surface antigen MHC II was used, and quantified by cytofluorimetric analysis at the end of the activation protocol. While 90% of the IFN-γ activated cells were MHC II⁺, not activated cells were virtually all negative for MHC II (FIG. 4A). At the end of the activation protocol, activated and control HMC3 cells were collected, total RNA extracted, reverse-transcribed into cDNA and used as a template for downstream gene expression analysis. We performed three duplex TaqMan real-time PCR assays specific for the endogenous human TSPO, endogenous human HLA-DRA and the LV transcript (WPRE), coupled to the human Glyceraldehyde 3-Phosphate Dehydrogenase (GAPDH) as a reference gene (N=2 experiments, run in duplicate). The results show that transcriptional level of the endogenous HLA-DRA gene increased from virtually zero in mock-activated (resting) HMC3 cells to approximately 5% of the GAPDH in all the activated cell samples (FIG. 4B), confirming the cytofluorimetric results. The transcriptional level of the endogenous TSPO gene increased from approximately 1.8% of the GAPDH in mock-activated (resting) HMC3 cells to approximately 2.6% of the GAPDH in all the activated cell samples (FIG. 4C).

As expected, lentiviral transcripts were absent in mock-transduced cells, independently from the resting/activation status. Cells transduced with the control LV hPGK-GRN showed a high basal lentiviral transcriptional level (FIG. 5A), corresponding to approximately the 25% of GAPDH, which increased up to −35% upon INFγ stimulation. The hTSPO promoter showed a minimal basal transcriptional activity, corresponding to ˜1.5% of GAPDH, which increased up to ˜3% of GAPDH upon INFγ stimulation (FIG. 5A). Notably, the hHLA promoter showed a minimal basal transcriptional activity, corresponding to˜2% of GAPDH (FIG. 5A), which increased up to ˜13% of GAPDH upon INFγ stimulation equivalent to a 6.6-fold increase (FIG. 5B). This indicates a good responsiveness of the synthetic hHLA promoter to INFγ stimulation, reproducing the transcriptional regulatory profile of the endogenous HLA-DRA promoter.

(iv) Evaluation of hGRN Protein Expression and Secretion in LV-Transduced HMC3 Cells Before and After Cell-Activation

Total protein extracts were prepared from the same cells previously analysed for mRNA expression and used for Western Blot (WB) analysis of human GRN protein content (FIGS. 6A and B). Comparable levels of total protein extracts were used, as indicated by GAPDH bands of comparable size in all the tested samples (FIG. 6A, bottom), while the band corresponding to the human GRN changed intensity in different samples: mock-transduced cells showed a minimal signal, corresponding to the basal endogenous expression of hGRN in HMC3 cells; cells transduced with the hPGK-GRN showed a very intense signal, which remained unchanged in resting and INFγ stimulated cells, confirming a high and constitutive gene expression driven by this promoter; cells transduced with the hTSPO-GRN did not show relevant differences with mock-transduced cells; and finally, cells transduced with the hHLA-GRN LV showed a consistent increase of hGRN protein quantity upon cell stimulation with INFγ. These results are in line with the mRNA expression analysis. Densitometric analysis of the WB bands (FIG. 6B) allowed a clear quantification of the WB signals, indicating that hGRN protein production increased ˜5-fold in cells transduced with hHLA-GRN upon INFγ stimulation (FIG. 6B). On the contrary, no relevant changes in GRN expression could be detected in the cells mock-transduced or transduced with the control LV, upon INFγ stimulation (FIG. 6B).

### Example 4. HSPC Transplantation in FTD Mice to Achieve Brain Delivery of hGRN

(i) LV Transduction of Wild-Type Murine Lineage Negative (Lin⁻) HSPCs—Preliminary MOI Test

Lineage-negative (Lin⁻) HSPCs were collected and purified from the bone marrow (BM) of wild-type C57BL/6J mice. First, to define the optimal transduction conditions for in vivo transplantation experiments, cells were transduced with the hPGK-GRN, the hTSPO-GRN or the hHLA-GRN LV, for 16 hours at increasing MOIs (25, 50, 75). After transduction, cells were washed in PBS and maintained in liquid culture (LC) with a standard cytokine cocktail. After two weeks in culture, mock- and LV-transduced cells were collected, and the genomic DNA was extracted for VCN determination by ddPCR. Although the different LVs showed a variable infectivity in Lin⁻ cells, they were all able to efficiently transduce the relevant target cells, demonstrating linearity between MOI and VCN (FIG. 7A). Importantly, the capacity of Lin⁻ cells to release hGRN following LV transduction was confirmed with all vectors, as demonstrated by ELISA assay on medium collected from 12 days LCs (FIG. 7B)

(ii) ICV transplantation of LV-transduced GRN^{+/-} and GRN^{-/-} HSPCs in FTD mice

To test and compare the capability of the three promoters (hPGK, hTSPO and hHLA) to drive hGRN expression in vivo in the clinically-relevant cell-type, organ and disease model, we investigated a Hematopoietic Stem/Progenitor Cell (HSPC) transplantation assay in FTD mice. In particular, we tested the capacity of Lin⁻ murine HSPCs derived from FTD mice and transduced with the three hGRN-encoding LVs (hPGK-GRN, hTSPO-GRN and hHLA-GRN LVs) to efficiently deliver the therapeutic hGRN protein to the brain of FTD mice, following intracerebroventricular (ICV) transplantation and upon engraftment, proliferation and differentiation. The three LVs were tested at two different vector doses (low vs high) based on the VCN achieved in transduced HSPCs that were transplanted.

Lin⁻ HSPCs were collected from the bone marrow (BM) of GRN^{+/-} or GRN^{-/-} donor mice and transduced with the hPGK-GRN, hTSPO-GRN or hHLA-GRN LV, for 16 hours at different MOIs based on the preliminary MOI test performed at step (i). After transduction, cells were washed and transplanted into fully-myeloablated young adult (8-weeks-old) GRN^{+/-} or GRN^{-/-} recipient mice (N=2-5 per group) by intracerebroventricular (ICV) administration. The control group (N=2) received Lin⁻ HSPCs that were previously transduced with a LV encoding GFP under control of the hPGK promoter. FIG. 8 shows a summary of the cell products and treatment groups.

TABLE 1
HSPC transplantation in FTD mice: summary of treatment groups

| | Promoter to | | | |
|---|---|---|---|---|
| | induce GRN | Transgene dose | | |
| Group Name | expression | (VCN) | MOI | N of mice |
| TSPO-GRN | hTSPO | 1-2 | 8 | 5 |
| low | | | | |
| TSPO-GRN | hTSPO | 7-8 | 30 | 4 |
| high | | | | |
| HLA-GRN | hMHC-II | 1-2 | 5 | 5 |
| low | | | | |
| HLA-GRN | hMHC-II | 7-8 | 15 | 5 |
| high | | | | |
| PGK-GRN | hPGK | 1-2 | 25 | 2 |
| low | | | | |
| PGK-GRN | hPGK | 7-8 | 85 | 2 |
| high | | | | |
| PGK-GFP | hPGK | 7-8 | 15 | 2 |
| UT WT | — | — | | 3 |

Table summarising the transplantation groups and the MOI used to transduce Lin⁻ cells with the specific LV in order to define the transgene dose.

For each transplantation experiment, a fraction of the cells was maintained in liquid culture two weeks for VCN determination, while another fraction was plated in Methocult to assess the clonogenic potential of the transduced HSPCs by the colony forming cell (CFC) assay. Five days after ICV cell-administration, transplanted mice intravenously received total BM cells obtained from FTD mice to support the systemic hematopoietic reconstitution.

Forty-two days post-transplantation, control and treated mice were sacrificed by intra-cardiac perfusion with saline and brains collected. The perfused brains were differentially processed to assess: i) engraftment efficiency, as estimated by VCN on myeloid enriched single cell homogenates; ii) hGRN content by ELISA on total protein extracts; and iii) lysosomal size/abundance by immunofluorescence on frozen tissue sections. For the control mice transplanted with HSPCs transduced with the GFP-expressing LV, part of the brain was processed to a single cell homogenate to be analysed for GFP expression by cytofluorimetry.

Although with some variability, we could observe HSPC engraftment (as estimated by VCN on brain) independently from the vector (hPFGK-GRN vs hTSPO-GRN vs hHLA-GRN) or the transgene dose (low vs high) (Table 2).

TABLE 2
Summary table showing HSPC VCN, brain VCN and % of
engraftment estimation. The engraftment estimation
is calculated based on the VCN of transplanted HSPCs
and the VCN in the brain of treated mice.

| | | | | Estimated |
|---|---|---|---|---|
| Group | Genotype | HSPC VCN | Brain VCN | engraftment % |
| TSPO HIGH | Ho | 12.7 | na | na |
| | Ho | 12.7 | 0.78 | 60 |
| | He | 7.8 | 0.00 | 0.4 |
| | He | 7.8 | 0.00 | 0.1 |
| TSPO LOW | Ho | 2.7 | 0.07 | 26.7 |
| | Ho | 2.7 | 0.03 | 11.2 |
| | Ho | 2.7 | 0.14 | 50.7 |
| | He | 2.1 | 0.00 | 1.5 |
| | He | 2.1 | 0.11 | 51.3 |
| HLA HIGH | Ho | 7.6 | 0.02 | 3.0 |
| | Ho | 7.6 | 0.02 | 2.1 |
| | Ho | 7.6 | 0.33 | 43.4 |
| | He | 7.9 | 0.01 | 1.1 |
| | He | 7.9 | 0.20 | 25.0 |
| HLA low | Ho | 2.9 | 0.09 | 29.9 |
| | Ho | 2.9 | 0.16 | 56.2 |
| | Ho | 2.9 | 0.26 | 89.4 |
| | He | 1.09 | 0.00 | 3.9 |
| | He | 1.09 | 0.00 | 2.5 |
| PGK HIGH | Ho | 8.4 | 0.09 | 11.2 |
| | Ho | 5 | 0.31 | 62.9 |
| PGK LOW | Ho | 2.4 | 0.05 | 19.1 |
| | Ho | 2.4 | 0.17 | 72.9 |
| GFP | Ho | 6.5 | 0.34 | 51.7 |
| | Ho | 6.6 | 0.13 | 20.0 |
| wt | wt | — | 0.02 | — |
| | wt | — | 0.00 | — |
| | wt | — | 0.01 | — |

Successful engraftment was also observed in mice that received HSPCs transduced with the PGK-GFP LV (FIG. 8A). Notably, in mice with a successful engraftment of donor cells, we were able to detect hGRN at a level comparable to the engraftment efficiency and to the VCN of the transplanted GRN-transduced HSPCs (FIG. 8B). As shown, the strong and constitutive hPGK promoter resulted in a higher hGRN expression in vivo compared to the two inducible novel promoters, in both the low and high dose groups. Of interest, the hGRN expression was well detectable and higher in the hHLA-high group compared to the hTSPO-high group, with levels decreased in the hHLA-low group as expected. In the hTSPO-low group no hGRN signal could be detected.

Overall, the in vivo data confirmed that upon ICV transplantation, GRN-transduced HSPCs are able to engraft and repopulate the brain myeloid compartment of myeloablated FTD mice, and to deliver hGRN locally, and that the delivery of the therapeutic hGRN into the brain of recipient mice is proportional to the degree of brain engraftment, to the transgene dose and can be controlled in a regulated fashion reflecting the nature of the promoter that drives transgene expression.

1. A population of modified hematopoietic stem and progenitor cells (HSPCs) where the cells carry one or more exogenous copies of a nucleic acid encoding progranulin, optionally wherein the progranulin is human progranulin.

2. (canceled)

3. The population of modified HSPCs of claim 1, wherein the cells express progranulin.

4. The population of modified HSPCs of claim 1, wherein the nucleic acid is a genomic DNA or a complementary DNA (cDNA).

5. The population of modified HSPCs of claim 1, wherein
(i) the nucleic acid encoding progranulin comprises the sequence of SEQ ID NO: 1, or a sequence with 95% sequence identity thereto; and/or
(ii) the progranulin encoded by the exogenous copy of a nucleic acid comprises the sequence of SEQ ID NO: 2, or a sequence with 95% sequence identity thereto.

6. (canceled)

7. The population of modified HSPCs of claim 1, wherein the nucleic acid is integrated into the genome of the cells via a viral vector or gene editing, wherein the nucleic acid is introduced into the cells via a viral vector, such as a lentiviral vector.

8. (canceled)

9. The population of modified HSPCs of claim 7, wherein the lentiviral vector comprises the GRN cDNA under
(i) the control of the human phosphoglycerate kinase (h PGK) promoter, or
(ii) under the control of an inflammatory cytokine inducible promoter, such as the human HLA-DRA promoter.

10. (canceled)

11. The population of modified HSPCs of claim 1, wherein:
a) the HSPC also comprises an exogenously introduced nucleic acid that encodes a metallothionein, such as Metallothionein 1G; and/or
b) the HSPC cells are mammalian cells, such as human cells; and/or
c) prior to the modification, the HSPCs are obtained from the bone marrow, umbilical cord, amniotic fluid, chorionic villi, cord blood, placental blood or peripheral blood; and/or
d) the HSPCs are derived from a healthy individual or are derived from an individual with a diagnosed disease or disorder; and/or
e) the HSPC cells are ex vivo cultured before or after or both before and after the introduction of the one or more exogenous copies of a nucleic acid encoding progranulin.

12.-17. (canceled)

18. The population of modified HSPCs of claim 1, wherein the HSPC cells are produced by a method comprising:
a) contacting a sample of unmodified HSPCs with a nucleic acid carrying an exogenous copy of a nucleic acid encoding progranulin; and
b) allowing the nucleic acid encoding progranulin to integrate into the genome of the cells so as to produce a population of modified HSPCs; optionally wherein the method further comprises establishing that there is at least one-fold increase in the amount of progranulin expressed in the modified cells compared to non-modified cells.

19. (canceled)

20. An ex vivo method for producing a population of hematopoietic stem cells (HSPCs) modified to express progranulin, the method comprising:
a) contacting a sample of HSPCs with a vector carrying an exogenous copy of a nucleic acid encoding progranulin, and optionally an exogenous copy of a nucleic acid encoding metallothionein, under conditions to allow the vector to transduce the HSPCs; and
b) culturing the cells in step (a) to produce a population of modified HSPCs cells expressing progranulin, and optionally also metallothionein, optionally wherein before, during or after contact with the viral vector the HSPCs are cultured in a medium comprising one or more transduction enhancers.

21. (canceled)

22. The method of claim 20, wherein
a) the method further comprises establishing that there is at least one-fold increase in the number of progranulin expressing cells compared to non-modified cells; and/or
b) the sample of HSPC is obtained from bone marrow, umbilical cord, amniotic fluid, chorionic villi, cord blood, placental blood or peripheral blood; and/or
c) the sample of HSPC is obtained from mobilized peripheral blood or bone marrow; and/or
d) the sample of HSPCs is obtained from a healthy individual or an individual with FTD; and/or
e) the HSPCs in the sample are CD34+ and/or CD45+; and/or
f) the vector is viral vector, such as a lentiviral vector; and/or
g) the nucleic acid(s) is/are integrated into the genome of the cells.

23.-29. (canceled)

30. A population of progranulin expressing HSPCs produced by the method of claim 20.

31. (canceled)

32. A composition comprising the HSPCs of claim 1.

33. (canceled)

34. A method of treating or preventing a neurodegenerative disorder mediated by aberrant expression of progranulin, such as frontotemporal dementia (FTD) or neuronal ceroid lipofuscinosis (NCL), in a subject in need thereof, the method comprising administering to the subject the population of modified HSPC cells of claim 1.

35. (canceled)

36. The method of claim 34, wherein the introduction of the HSPCs results in an increase in progranulin levels in brain tissue, in particular in microglia.

37. The method of claim 34, wherein the HSPCs are:
(i) autologous to the recipient subject;
(ii) non-autologous and allogenic to the recipient subject; or
(iii) non-autologous and xenogeneic to the recipient subject.

38. The method of claim 37, wherein the HSPCs are administered to the patient via intravenous (IV) and/or intracerebroventricular (ICV) and/or intrathecal lumbar (ITL) injection and/or intra cisterna magna (ICM).

39.-40. (canceled)

41. A method of preventing or treating a disease mediated by a dysfunctional gene encoding progranulin, the method comprising:
(i) determining whether a subject has a mutation in the progranulin gene that leads to aberrant expression or amount of progranulin; and,
(ii) if the subject has a mutation in the progranulin gene that leads to aberrant expression or amount of progranulin they are administered the modified HSPCs of claim 1, or a composition comprising such population of HSPCs.

42. A composition comprising the HSPCs of claim 30.

43. A method of treating or preventing a neurodegenerative disorder mediated by aberrant expression of progranulin, such as frontotemporal dementia (FTD) or neuronal ceroid lipofuscinosis (NCL), in a subject in need thereof, the method comprising administering to the subject the population of modified HSPC cells of claim 30.

44. A method of treating or preventing a neurodegenerative disorder mediated by aberrant expression of progranulin, such as frontotemporal dementia (FTD) or neuronal ceroid lipofuscinosis (NCL), in a subject in need thereof, the method comprising administering to the subject the composition of claim 32.


SEQUENCE LISTING (9 sequences)

SEQ ID NO: 1 - DNA, 1782 bp, Homo sapiens
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 2 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 3 - protein, 62 aa, Homo sapiens
MDPNCSCAAAGVSCTCASSCKCKECKCTSCKKSCCSCCPVGCAKCAQGCICKGASEKCSCCA

SEQ ID NO: 4 - DNA, 420 bp, Homo sapiens
actccgccttccacgtgcacccactgcctcttcccttctcgcttgggaactctagtctcgcctcgggttgcaatggaccccaactgctcctgtgccgctggtgtctcctgcacctgcgccagctcctgcaagtgcaaagagtgcaaatgcacctcctgcaagaagagctgctgctcctgctgccctgtgggctgtgccaagtgtgcccagggctgcatctgcaaaggggcatcggagaagtgcagctgctgcgcctgatgtcgggacagccctgctcccaagtacaaatagagtgacccgtaaaatccaggattttttgttttttgctacaatcttgacccctttgctacattcctttttttctgtgaaatatgtgaataataattaaacacttagacttgaaaaaaaaaaaaaaaaaaa

SEQ ID NO: 5 - DNA, 1788 bp, Mus musculus
ggatccgccaccatgtgggtcctgatgagctggctggccttcgcggcagggctggtagccggaacacagtgtccagatgggcagttctgccctgttgcctgctgccttgaccagggaggagccaactacagctgctgtaaccctcttctggacacatggcctagaataacgagccatcatctagatggctcctgccagacccatggccactgtcctgctggctattcttgtcttctcactgtgtctgggacttccagctgctgcccgttctctaagggtgtgtcttgtggtgatggctaccactgctgcccccagggcttccactgtagtgcagatgggaaatcctgcttccagatgtcagataaccccttgggtgctgtccagtgtcctgggagccagtttgaatgtcctgactctgccacctgctgcattatggttgatggttcgtggggatgttgtcccatgccccaggcctcttgctgtgaagacagagtgcattgctgtccccatggggcctcctgtgacctggttcacacacgatgcgtttcacccacgggcacccacaccctactaaagaagttccctgcacaaaagaccaacagggcagtgtctttgcctttttctgtcgtgtgccctgatgctaagacccagtgtcccgatgattctacctgctgtgagctacccactgggaagtatggctgctgtccaatgcccaatgccatctgctgttccgaccacctgcactgctgcccccaggacactgtatgtgacctgatccagagtaagtgcctatccaagaactacaccacggatctcctgaccaagctgcctggatacccagtgaaggaggtgaagtgcgacatggaggtgagctgccctgaaggatatacctgctgccgcctcaacactggggcctggggctgctgtccatttgccaaggccgtgtgttgtgaggatcacattcattgctgcccggcagggtttcagtgtcacacagagaaaggaacctgcgaaatgggtatcctccaagtaccctggatgaagaaggtcatagcccccctccgcctgccagacccacagatcttgaagagtgatacaccttgtgatgacttcactaggtgtcctacaaacaatacctgctgcaaactcaattctggggactggggctgctgtcccatcccagaggctgtctgctgctcagacaaccagcattgctgccctcagggcttcacatgtctggctcaggggtactgtcagaagggagacacaatggtggctggcctggagaagatacctgcccgccagacaaccccgctccaaattggagatatcggttgtgaccagcataccagctgcccagtagggcaaacctgctgcccaagcctcaagggaagttgggcctgctgccagctgccccatgctgtgtgctgtgaggaccggcagcactgttgcccggccgggtacacctgcaatgtgaaggcgaggacctgtgagaaggatgtcgattttatccagcctcccgtgctcctgaccctcggccctaaggttgggaatgtggagtgtggagaagggcatttctgccatgataaccagacctgttgtaaagacagtgcaggagtctgggcctgctgtccctacctaaagggtgtctgctgtagagatggacgtcactgttgccccggtggcttccactgttcagccaggggaaccaagtgtttgcgaaagaagattcctcgctgggacatgtttttgagagatccggtcccaagaccgctactgtaagtcgac

SEQ ID NO: 6 - DNA, 1800 bp, Artificial Sequence
  exemplary codon-optimised human progranulin nucleic acid sequence
ggatccgccaccatgtggacactggtcagctgggtggccctcacagctggactggtggctggcacaagatgtcccgacggccagttttgccccgtggcttgttgcctcgaccccggaggcgcttcctacagctgctgtagacctctgctggataaatggcctacaacactgagcagacatctgggcggcccttgccaagtcgatgctcattgctccgccggccacagctgcatcttcacagtgtccggaacatccagctgctgcccctttcccgaagctgtcgcttgcggagacggccatcactgctgtcctagaggctttcactgcagcgctgacggcagaagctgcttccagaggagcggaaacaacagcgtgggagccattcaatgtcccgacagccagttcgaatgccccgactttagcacatgctgcgtgatggtggacggcagctggggatgttgccccatgccccaagccagctgctgcgaggatagagtccactgttgtccccacggagctttttgcgatctggtgcatacaagatgcatcacccccaccggcacccaccctctggccaagaaactgcccgctcagaggacaaatagagctgtggctctgagcagcagcgtcatgtgtcccgacgctaggtctagatgccccgacggctccacatgctgtgaactgccctccggcaagtatggctgctgccctatgcccaatgctacatgctgctccgatcatctgcattgctgcccccaagacaccgtgtgtgatctgattcagagcaagtgcctctccaaggagaacgccaccacagatctgctgaccaagctccccgcccacacagtgggcgatgtgaagtgtgacatggaggtgagctgccccgacggatacacatgctgtaggctgcagtccggcgcttggggatgctgtcccttcacccaagccgtgtgctgtgaagaccatatccattgttgccccgctggattcacatgcgacacccaaaagggcacatgcgagcaaggccctcatcaagtgccttggatggagaaagcccccgctcatctgtctctgcccgatccccaagctctgaagagggacgtgccttgcgataacgtgagcagctgcccttccagcgacacatgctgtcaactgaccagcggagagtggggctgttgccctatccccgaggctgtctgctgtagcgaccaccagcactgttgtcctcaaggatacacatgcgtggccgaaggacaatgtcagaggggctccgagatcgtggctggactcgagaagatgcccgccagaagagcctctctgagccatcctagagacatcggatgcgaccagcatacaagctgccccgtgggccagacatgttgcccctctctgggcggaagctgggcttgttgccaactcccccacgccgtctgttgtgaggatagacaacattgctgtcccgccggctacacatgcaacgtcaaggccagaagctgcgaaaaggaggtggtgtccgcccagcccgccacatttctggctagatccccccatgtgggcgtgaaggatgtggagtgcggagagggacacttttgccacgacaaccagacatgctgcagagacaataggcaaggctgggcttgctgcccttataggcaaggcgtgtgctgtgctgatagaaggcactgttgccccgccggcttcagatgcgccgccagaggcaccaaatgtctgagaagagaggctcctaggtgggacgctcctctgagagatcccgctctgaggcagctgctgtgagtcgac

SEQ ID NO: 7 - DNA, 537 bp, Homo sapiens
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcg

SEQ ID NO: 8 - DNA, 361 bp, Artificial Sequence
  synthetic HLA-DRA derived promoter
tcgatttccgaattccactatccctgtctagaagtcagattggggttaaagagtctgtccgtgattgactaacagtcttaaatacttgatttgttgttgttgttgtcctgtttgtttaagaactttacttctttatccaatgaacggagtatcttgtgtcctggaccctttgcaagaacccttcccctagcaacagatgcgtcatctcaaaatatttttctgattggccaaagagtaattgatttgcattttaatggtcagactctattacaccccacattctcttttcttttattcttgtctgttctgcctcactcccgagctctactgactcccaacagagcgcccaagaagaaaacgc

SEQ ID NO: 9 - DNA, 634 bp, Artificial Sequence
  MPP01(hTSPO_prximal5′prom)(“P1”)
tgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgtccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccg